(19) United States
(12) Patent Application Publication
(10) Pub. No.: US 2013/0129675 A1
(43) Pub. Date: May 23, 2013

(54) INTERFERON THERAPIES IN COMBINATION WITH BLOCKADE OF STAT3 ACTIVATION

(71) Applicants: Waldemar Priebe, Houston, TX (US); Amy Heimberger, Houston, TX (US); Izabela Fokt, Houston, TX (US); Jayakmuar Arumugam, Sugarland, TX (US); Ling-Yuan Kong, Houston, TX (US); Timothy Madden, Sugarland, TX (US); Charles Conrad, Spring, TX (US)

(72) Inventors: Waldemar Priebe, Houston, TX (US); Amy Heimberger, Houston, TX (US); Izabela Fokt, Houston, TX (US); Jayakmuar Arumugam, Sugarland, TX (US); Ling-Yuan Kong, Houston, TX (US); Timothy Madden, Sugarland, TX (US); Charles Conrad, Spring, TX (US)

(73) Assignee: BOARD OF REGENTS, THE UNIVERSITY OF TEXAS SYSTEM, Austin, TX (US)

(21) Appl. No.: 13/513,549

(22) PCT Filed: Dec. 4, 2010
(86) PCT No.: PCT/US2010/059005

§ 371 Date: Jun. 1, 2012

Related U.S. Application Data
(60) Provisional application No. 61/266,812, filed on Dec. 4, 2009.

(51) Int. Cl. A61K 38/21; A61K 31/165; A61K 31/706; A61K 31/4164; A61K 45/06; A61K 31/216; A61K 31/44; A61K 31/277

(52) U.S. Cl. 424/85.4

(57) ABSTRACT

Methods of modulating the INF-induced STAT3 activation in a patient in need-thereof are provided for the treatment of disease. The methods comprise the step of administrating to a patient the combination of a therapeutically effective amount of interferon including INF-α and/or INF-β in combination with a STAT3 inhibitor.

### CROSS REFERENCE TO RELATED APPLICATIONS

This application claims the benefit of U.S. provisional patent application No. 61/266,812, filed Dec. 4, 2009, the entire contents of which are incorporated herein by reference.

### STATEMENT REGARDING FEDERALLY SPONSORED RESEARCH OR DEVELOPMENT

None.

### THE NAMES OF THE PARTIES TO A JOINT RESEARCH AGREEMENT

None.

### FIELD OF INVENTION

The present invention relates to improved dosing regimens for the treatment cancer and other diseases via the use of interferons with a STAT3 inhibitor that reduce the side effect profile of the interferon, while enhancing the efficacy of the interferon treatment.

### BACKGROUND OF THE INVENTION

Interferons (IFNs) are mammalian cytokines that exhibit antiviral and anticancer activities. Interferons are classified as Type I or II based on receptor complex recognition and cellular origin. Interferon-α and interferon-β (Type 1 IFNs) exert their antiviral and anticancer activity through activation of STAT1. Type I interferon (IFN)-α is known to have powerful effects on immune cells, including the inducement of dendritic cell maturation, enhancement of T-cell survival, and induction of immunological memory. IFN-α is also being used in non-cancer related therapies. However, during such therapies IFN-α is considered to be a major factor associated with inducing outbursts of psoriasis. Moreover, several large, cooperative-group adjuvant trials have documented a 25-33% reduction in the relative relapse risk with the use of high-dose type I IFN-α for stage II and III melanoma with overall survival prolongation. Furthermore, by contrast, no large cooperative-group trials have shown significant prolongation of survival with type I IFN-α for inoperable stage IV melanoma.

Similarly, STAT3 inhibitors have not been shown to induce sufficient immunological memory to be fully protective against every tumor re-challenge. Kolumam G A, Thomas S, et al., Type I Interferons Act Directly On CD8 T Cells To Allow Clonal Expansion And Memory Formation In Response To Viral Infection, J Exp Med 2005; 202:637-650. A key transcriptional factor, signal transducer and activator of transcription (STAT) 3, drives the fundamental components of tumor malignancy and metastases in many parts of the body including the Central Nervous System (“CNS”). STAT3 promotes tumorigenesis by enhancing proliferation, angiogenesis, invasion, metastasis, and immunosuppression. While a group of potent, small molecule inhibitors of STAT3 display efficacy against malignancy with minimal toxicity in murine models, the mechanism of STAT3 blockade agents in vivo can be cytotoxic to the tumor and negatively impact the immune system. Therefore, methods of treating tumor malignancy and metastases are needed which will treat patients with malignancies while acting as immunotherapeutic enhancers.

### SUMMARY OF THE INVENTION

In one embodiment, the current invention includes a method of treating a proliferative disease comprising the step of administering to a patient a therapeutically effective amount of Type 1 interferon in combination with a STAT3 pathway inhibitor.

In one aspect, the STAT3 pathway inhibitor has structural Formula I:

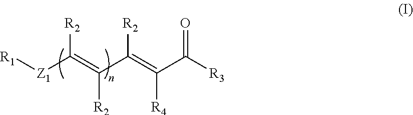

or a pharmaceutically acceptable salt thereof, wherein:

n is 0 or 1;

m is an integer selected from 1, 2, 3, or 4;

R₁ is selected from the group consisting of:

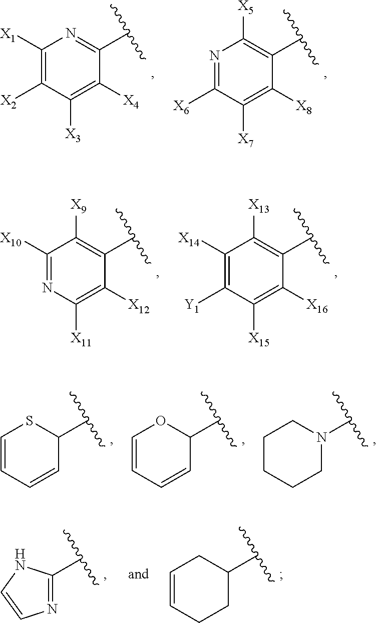

each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;

R₃ is selected from the group consisting of:

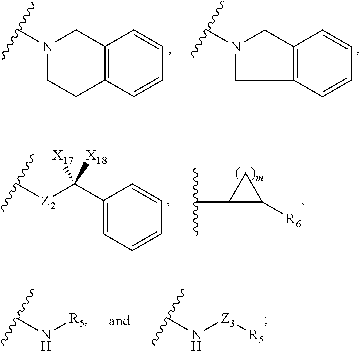

R₄ is selected from the group consisting of cyano, alkylamine, CH₂S-alkyl, alkyl, and CH₂N₃;

R₅ and R₆ are each independently selected from the group consisting of:

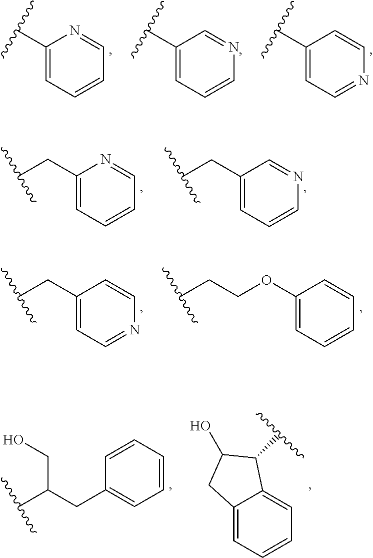

monosaccharide, polysaccharide, monosaccharide derivative, optionally substituted aryl, and optionally substituted arylalkyl;

X₁, X₂, X₃, X₄, X₅, X₆, X₇, X₈, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;

X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;

Y₁ is selected from the group consisting of hydroxyl, halogen, and nitro;

Z₁ is selected from the group consisting of alkyl and a bond;

Z₂ is selected from the group consisting of N H, S, and O; and

Z₃ is alkyl.

In another aspect of this method; R₁ is selected from the group consisting of:

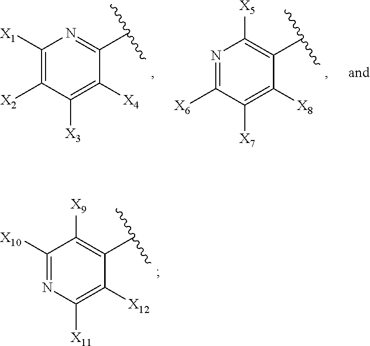

each instance of R₂ is hydrogen;

R₃ is

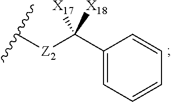

and

Z₂ is NH.

In another aspect of this method; X₁, X₂, X₃, X₄, X₅, X₆, X₇, X₈, X₉, X₁₀, X₁₁, and X₁₂ are each independently selected from the group consisting of hydrogen and halogen; and

X₁₇ and X₁₈ are each independently selected from the group consisting of hydrogen, alkyl, and cycloalkyl.

In another aspect of this method;

R₁ is

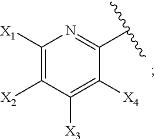

X₁ is halogen; and

X₂, X₃, and X₄ are hydrogen.

In another aspect of this method; one of X₁₇ and X₁₈ is hydrogen;
the other of one of X₁₇ and X₁₈ is selected from the group consisting of hydrogen, methyl, ethyl, and cyclopropyl.

In another aspect of this method; n is 0.

In another aspect of this method; n is 1.

In another aspect of any of these methods, the STAT3 pathway inhibitor is selected from the group of compounds consisting of Examples 1-63.

In another aspect of any of these methods, the proliferative disease is selected from the group consisting of psoriasis, skin cancer, CNS cancer including brain cancer and cancer metastatic to CNS, ovarian cancer, head cancer and neck cancer, prostate cancer, hematological malignancies including leukemia, lymphoma and myeloma, and breast cancer. In one aspect the proliferative disease is skin cancer. In one aspect, the skin cancer is selected from the group consisting squamous cell carcinomas, basal cell cancers, cutaneous T-cell lymphomas, primary cutaneous B cell lymphomas, Dermatofibrosarcoma protuberans, Merkel cell carcinoma, Kaposi's sarcoma, keratoacanthoma, and melanoma. In one aspect of any of these methods, the proliferative disease is melanoma.

In another aspect of any of these methods, the melanoma is CNS melanoma. In another aspect of any of these methods, the patient has Leptomeningeal disease (LMD). In another aspect of any of these methods, the patient has stage III melanoma. In another aspect of any of these methods, the patient has stage IV melanoma.

In another aspect of any of these methods, combination of the STAT3 inhibitor and the Type 1 interferon is characterized by a synergistic response compared to either agent alone.

In another aspect of any of these methods, the proliferative disease is melanoma. In another aspect of any of these methods, the melanoma is CNS melanoma. In another aspect of any of these methods, patient has Leptomeningeal disease (LMD). In another aspect of any of these methods, the patient has stage III melanoma. In another aspect of any of these methods, the patient has stage IV melanoma. In another aspect of any of these methods, the patient has failed to substantially respond to at least one prior first tier cancer therapy.

In another aspect of any of these methods, the proliferative disease has been determined to comprise tissue in which pSTAT3 is phosphorylated at tyrosine 705. In another aspect of any of these methods, the proliferative disease has been determined to comprise tissue in which pSTAT3 is phosphorylated at serine 727.

In another aspect of any of these methods, the STAT3 pathway inhibitor blocks formation of STAT3 homodimers and heterodimers. In another aspect of any of these methods, the STAT3 pathway inhibitor blocks the nuclear translocation of STAT3 and its dimers. In another aspect of any of these methods, the STAT3 pathway inhibitor blocks STAT3 or its dimers or heterodimers DNA binding.

In another aspect of any of these methods, the STAT3 pathway inhibitor has a structural formula selected from the group consisting of:

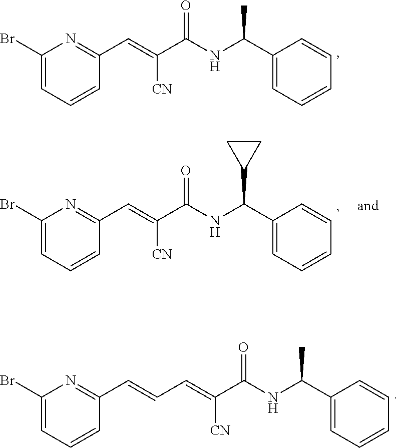 [SMILES: C[C@H](NC(=O)/C(C#N)=C/c1cccc(Br)n1)c1ccccc1 ; N#C/C(=C\c1cccc(Br)n1)C(=O)N[C@H](c1ccccc1)C1CC1 ; C[C@H](NC(=O)/C(C#N)=C/C=C/c1cccc(Br)n1)c1ccccc1]

In another aspect of any of these methods the STAT3 pathway inhibitor is administered topically. In another aspect of any of these methods the STAT3 pathway inhibitor is administered iv. In another aspect of any of these methods the STAT3 pathway inhibitor is administered p.o.

In another embodiment, the current invention includes a method of potentiating the activity of Type 1 interferon for treatment of a proliferative disease comprising the step of administering to a patient a therapeutically effective amount of Type 1 interferon in combination with a STAT3 pathway inhibitor.

In one aspect of this method, the STAT3 pathway inhibitor has structural Formula I:

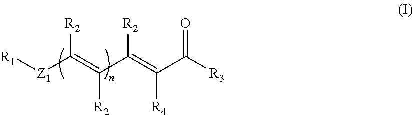

or a pharmaceutically acceptable salt thereof, wherein:

n is 0 or 1;

m is an integer selected from 1, 2, 3, or 4;

R₁ is selected from the group consisting of:

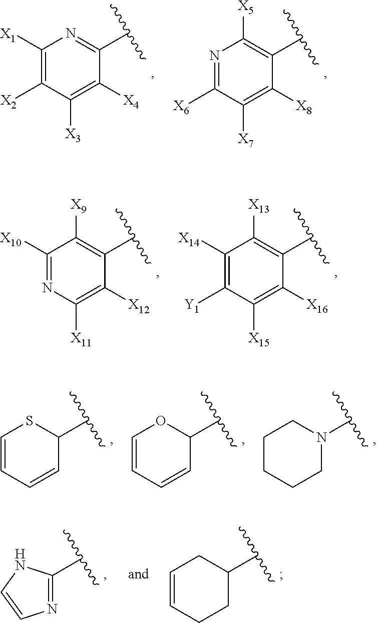

each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;

R₃ is selected from the group consisting of:

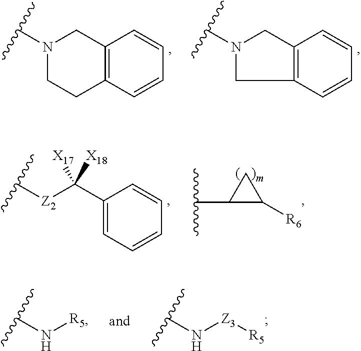

R₄ is selected from the group consisting of cyano, alkylamine, CH₂S-alkyl, alkyl, and CH₂N₃;

R₅ and R₆ are each independently selected from the group consisting of:

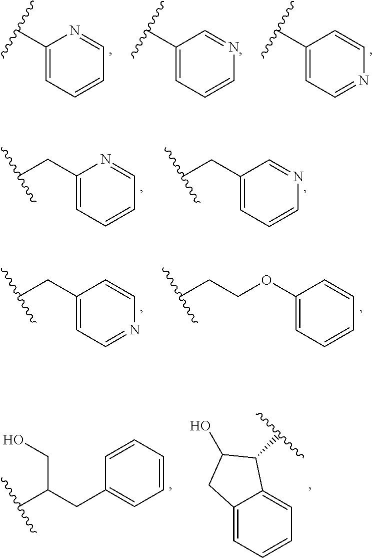

monosaccharide, polysaccharide, monosaccharide derivative, optionally substituted aryl, and optionally substituted arylalkyl;

X₁, X₂, X₃, X₄, X₅, X₆, X₇, X₈, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;

X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;

Y₁ is selected from the group consisting of hydroxyl, halogen, and nitro;

Z₁ is selected from the group consisting of alkyl and a bond;

Z₂ is selected from the group consisting of N H, S, and O; and

Z₃ is alkyl.

In another aspect of this method; R₁ is selected from the group consisting of:

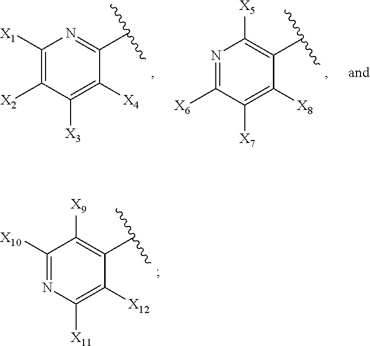

each instance of R₂ is hydrogen;

R₃ is

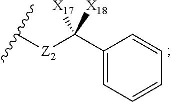

and

Z₂ is NH.

In another aspect of this method; X₁, X₂, X₃, X₄, X₅, X₆, X₇, X₈, X₉, X₁₀, X₁₁, and X₁₂ are each independently selected from the group consisting of hydrogen and halogen; and

X₁₇ and X₁₈ are each independently selected from the group consisting of hydrogen, alkyl, and cycloalkyl.

In another aspect of this method;

R₁ is

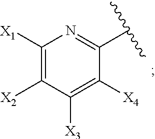

X₁ is halogen; and

X₂, X₃, and X₄ are hydrogen.

In another aspect of this method; one of X₁₇ and X₁₈ is hydrogen;

the other of one of X₁₇ and X₁₈ is selected from the group consisting of hydrogen, methyl, ethyl, and cyclopropyl.

In another aspect of this method; n is 0.

In another aspect of this method; n is 1.

In another aspect of any of these methods, the STAT3 pathway inhibitor is selected from the group of compounds consisting of Examples 1-63.

In another aspect of any of these methods, the proliferative disease is selected from the group consisting of psoriasis, skin cancer, CNS cancer including brain cancer and cancer metastatic to CNS, ovarian cancer, head cancer and neck cancer, prostate cancer, hematological malignancies including leukemia, lymphoma and myeloma, and breast cancer. In one aspect the proliferative disease is skin cancer. In one aspect, the skin cancer is selected from the group consisting squamous cell carcinomas, basal cell cancers, cutaneous T-cell lymphomas, primary cutaneous B cell lymphomas, Dermatofibrosarcoma protuberans, Merkel cell carcinoma, Kaposi's sarcoma, keratoacanthoma, and melanoma.

In another aspect of any of these methods, the proliferative disease is melanoma. In another aspect of any of these methods, the melanoma is CNS melanoma. In another aspect of any of these methods, the patient has Leptomeningeal disease (LMD). In another aspect of any of these methods, the patient has stage III melanoma. In another aspect of any of these methods, the patient has stage IV melanoma.

In another aspect of any of these methods, the STAT3 pathway inhibitor potentiates the activity of the Type I interferon by greater than about 30%.

In another aspect of any of these methods, the proliferative disease has been determined to comprise tissue in which pSTAT3 is phosphorylated at tyrosine 705. In another aspect of any of these methods, wherein the proliferative disease has been determined to comprise tissue in which pSTAT3 is phosphorylated at serine 727 In another aspect of any of these methods, the STAT3 pathway inhibitor blocks formation of STAT3 homodimers and heterodimers. In another aspect of any of these methods, wherein the STAT3 pathway inhibitor blocks the nuclear translocation of STAT3 and its dimers. In another aspect of any of these methods, wherein the STAT3 pathway inhibitor blocks STAT3 or its dimers or heterodimers DNA binding.

In another aspect of any of these methods, the STAT3 inhibitor blocks the phosphorylation of STAT3 at tyrosine 705 and/or serine 727. In another aspect of any of these methods, the STAT3 inhibitor induces secondary processes inactivating pSTAT3. In another aspect of any of these methods, the STAT3 pathway inhibitor decreases levels of pSTAT3.

In another aspect of any of these methods, the STAT3 pathway inhibitor has a structural formula selected from the group consisting of:

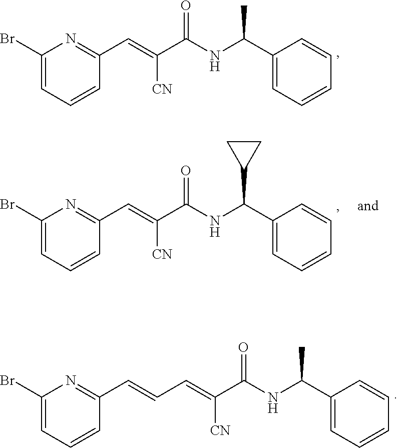 [SMILES: C[C@H](NC(=O)/C(C#N)=C/c1cccc(Br)n1)c1ccccc1 ; N#C/C(=C\c1cccc(Br)n1)C(=O)N[C@H](c1ccccc1)C1CC1 ; C[C@H](NC(=O)/C(C#N)=C/C=C/c1cccc(Br)n1)c1ccccc1]

In another aspect of any of these methods the STAT3 pathway inhibitor is administered topically. In another aspect of any of these methods the STAT3 pathway inhibitor is administered iv. In another aspect of any of these methods the STAT3 pathway inhibitor is administered p.o.

In another embodiment, the current invention includes a method of modulating IFN-induced STAT3 activation during anti-viral therapy with a type I interferon, comprising the step of administering to a patient a therapeutically effective amount of Type 1 interferon in combination with a STAT3 pathway inhibitor, wherein the STAT3 pathway inhibitor reduces the severity of at least one side effect of the Type 1 interferon.

In one aspect of this method, the STAT3 pathway inhibitor has structural Formula I:

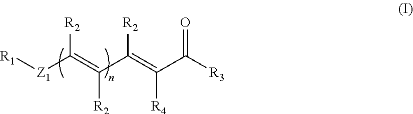

or a pharmaceutically acceptable salt thereof, wherein:

n is 0 or 1;

m is an integer selected from 1, 2, 3, or 4;

R₁ is selected from the group consisting of:

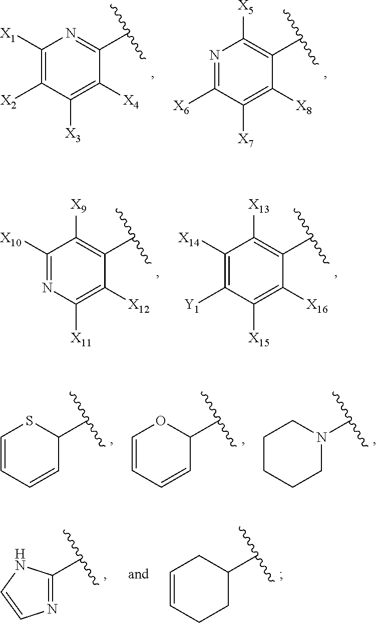

each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;

R₃ is selected from the group consisting of:

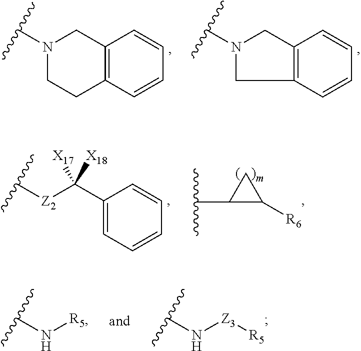

R₄ is selected from the group consisting of cyano, alkylamine, CH₂S-alkyl, alkyl, and CH₂N₃;

R₅ and R₆ are each independently selected from the group consisting of:

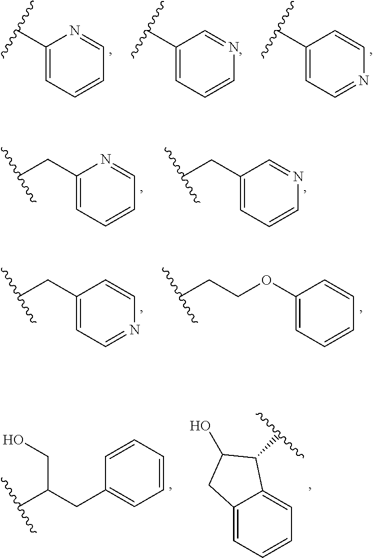

monosaccharide, polysaccharide, monosaccharide derivative, optionally substituted aryl, and optionally substituted arylalkyl;

X₁, X₂, X₃, X₄, X₅, X₆, X₇, X₈, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;

X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;

Y₁ is selected from the group consisting of hydroxyl, halogen, and nitro;

Z₁ is selected from the group consisting of alkyl and a bond;

Z₂ is selected from the group consisting of N H, S, and O; and

Z₃ is alkyl.

In another aspect of this method; R₁ is selected from the group consisting of:

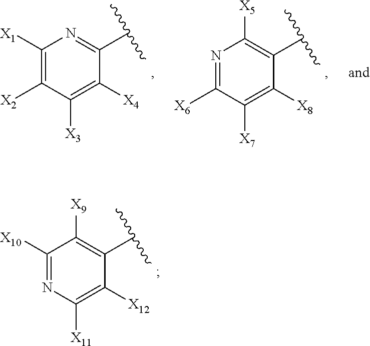

each instance of R₂ is hydrogen;

R₃ is

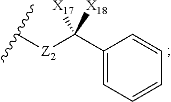

and

Z₂ is NH.

In another aspect of this method; X₁, X₂, X₃, X₄, X₅, X₆, X₇, X₈, X₉, X₁₀, X₁₁, and X₁₂ are each independently selected from the group consisting of hydrogen and halogen; and

X₁₇ and X₁₈ are each independently selected from the group consisting of hydrogen, alkyl, and cycloalkyl.

In another aspect of this method;

R₁ is

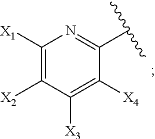

X₁ is halogen; and

X₂, X₃, and X₄ are hydrogen.

In another aspect of this method; one of X₁₇ and X₁₈ is hydrogen;

the other of one of X₁₇ and X₁₈ is selected from the group consisting of hydrogen, methyl, ethyl, and cyclopropyl.

In another aspect of this method; n is 0.

In another aspect of this method; n is 1.

In another aspect of any of these methods, the STAT3 pathway inhibitor is selected from the group of compounds consisting of Examples 1-63.

In another aspect of any of these methods, the side effect of the Type 1 interferon is selected from the group consisting of psoriasis, Crohn's disease, inflammatory bowel disease, and pulmonary fibrosis.

In another aspect of any of these methods, the STAT3 pathway inhibitor blocks formation of STAT3 homodimers and heterodimers. In another aspect of any of these methods, the STAT3 pathway inhibitor blocks the nuclear translocation of STAT3 and its dimers. In another aspect of any of these methods, the STAT3 pathway inhibitor blocks STAT3 or its dimers or heterodimers DNA binding. In another aspect of any of these methods, the STAT3 inhibitor blocks the phosphorylation of STAT3 at tyrosine 705 and/or serine 727. In another aspect of any of these methods, the STAT3 inhibitor induces secondary processes inactivating pSTAT3. In another aspect of any of these methods, the STAT3 pathway inhibitor decreases levels of pSTAT3.

In another aspect of any of these methods, the STAT3 pathway inhibitor has a structural formula selected from the group consisting of:

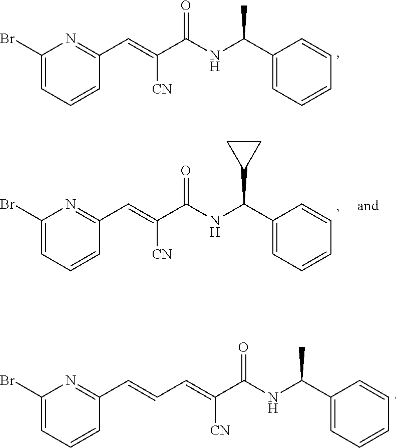 [SMILES: C[C@H](NC(=O)/C(C#N)=C/c1cccc(Br)n1)c1ccccc1 ; N#C/C(=C\c1cccc(Br)n1)C(=O)N[C@H](c1ccccc1)C1CC1 ; C[C@H](NC(=O)/C(C#N)=C/C=C/c1cccc(Br)n1)c1ccccc1]

In another aspect of any of these methods the STAT3 pathway inhibitor is administered topically. In another aspect of any of these methods the STAT3 pathway inhibitor is administered iv. In another aspect of any of these methods the STAT3 pathway inhibitor is administered p.o.

In another embodiment, the current invention includes a method of modulating IFN-induced STAT3 activation during treatment for viral hepatitis comprising the step of administering to a patient a therapeutically effective amount of Type 1 interferon in combination with a STAT3 pathway inhibitor.

In one aspect of this method, the STAT3 pathway inhibitor has structural Formula I:

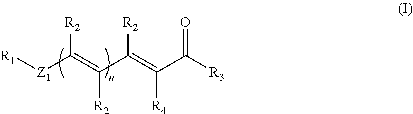

or a pharmaceutically acceptable salt thereof, wherein:

n is 0 or 1;

m is an integer selected from 1, 2, 3, or 4;

R₁ is selected from the group consisting of:

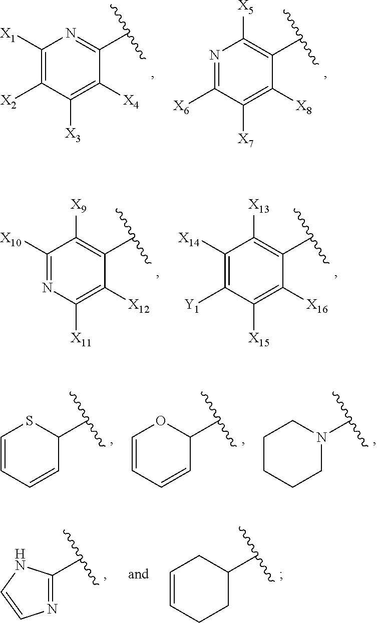

each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;

R₃ is selected from the group consisting of:

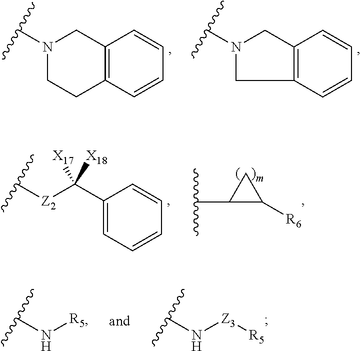

R₄ is selected from the group consisting of cyano, alkylamine, CH₂S-alkyl, alkyl, and CH₂N₃;

R₅ and R₆ are each independently selected from the group consisting of:

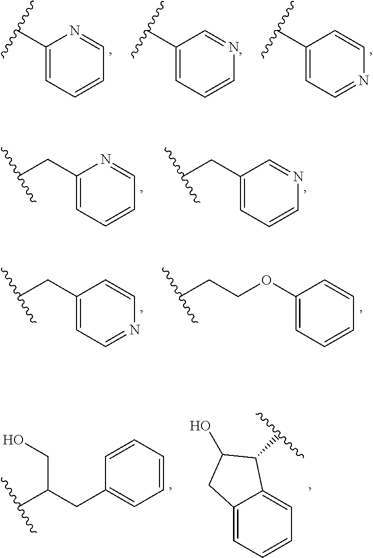

monosaccharide, polysaccharide, monosaccharide derivative, optionally substituted aryl, and optionally substituted arylalkyl;

X₁, X₂, X₃, X₄, X₅, X₆, X₇, X₈, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;

X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;

Y₁ is selected from the group consisting of hydroxyl, halogen, and nitro;

Z₁ is selected from the group consisting of alkyl and a bond;

Z₂ is selected from the group consisting of N H, S, and O; and

Z₃ is alkyl.

In another aspect of this method; R₁ is selected from the group consisting of:

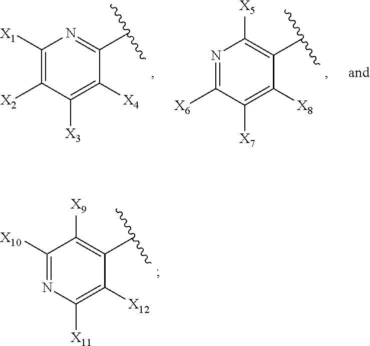

each instance of R₁ is hydrogen;

R₃ is

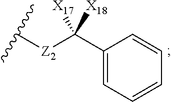

and

Z₂ is NH.

In another aspect of this method; X₁, X₂, X₃, X₄, X₅, X₆, X₇, X₈, X₉, X₁₀, X₁₁, and X₁₂ are each independently selected from the group consisting of hydrogen and halogen; and

X₁₇ and X₁₈ are each independently selected from the group consisting of hydrogen, alkyl, and cycloalkyl.

In another aspect of this method;

R₁ is

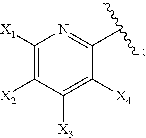

X₁ is halogen; and

X₂, X₃, and X₄ are hydrogen.

In another aspect of this method; one of X₁₇ and X₁₈ is hydrogen;

the other of one of X₁₇ and X₁₈ is selected from the group consisting of hydrogen, methyl, ethyl, and cyclopropyl.

In another aspect of this method; n is 0.

In another aspect of this method; n is 1.

In another aspect of any of these methods, the STAT3 pathway inhibitor is selected from the group of compounds consisting of Examples 1-63.

In another aspect of any of these methods, the side effect of the Type 1 interferon is selected from the group consisting of psoriasis, Crohn's disease, thyroiditis, autoimmune hepatitis, inflammatory bowel disease, and pulmonary fibrosis.

In another aspect of any of these methods, the STAT3 pathway inhibitor blocks formation of STAT3 homodimers and heterodimers. In another aspect of any of these methods, the STAT3 pathway inhibitor blocks the nuclear translocation of STAT3 and its dimers. In another aspect of any of these methods, the STAT3 pathway inhibitor blocks STAT3 or its dimers or heterodimers DNA binding. In another aspect of any of these methods, the STAT3 inhibitor blocks the phosphorylation of STAT3 at tyrosine 705 and/or serine 727. In another aspect of any of these methods, the STAT3 inhibitor induces secondary processes inactivating pSTAT3. In another aspect of any of these methods, the STAT3 pathway inhibitor decreases levels of pSTAT3.

In another aspect of any of these methods, the STAT3 pathway inhibitor has a structural formula selected from the group consisting of:

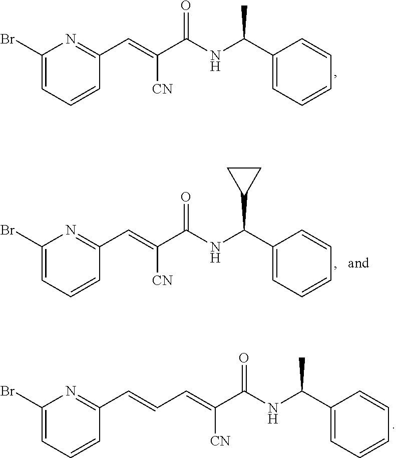 [SMILES: C[C@H](NC(=O)/C(C#N)=C/c1cccc(Br)n1)c1ccccc1 ; N#C/C(=C\c1cccc(Br)n1)C(=O)N[C@H](c1ccccc1)C1CC1 ; C[C@H](NC(=O)/C(C#N)=C/C=C/c1cccc(Br)n1)c1ccccc1]

In another aspect of any of these methods the STAT3 pathway inhibitor is administered topically. In another aspect of any of these methods the STAT3 pathway inhibitor is administered iv. In another aspect of any of these methods the STAT3 pathway inhibitor is administered p.o.

In another embodiment, the current invention includes a method of modulating anti-viral therapy with a type I interferon, comprising the step of administering to a patient a therapeutically effective amount of Type 1 interferon in combination with a Jak2 inhibitor, wherein the Jak2 pathway inhibitor reduces the severity of at least one side effect of the Type 1 interferon.

In one aspect of this method, wherein the Jak2 inhibitor is selected from the group consisting of INCB018424, TG101348, CEP-701 (lestaurtinib), AZD1480, XL019, CYT-387, SGI-1252, SB1518, tasocitinib (CP-690550), LY3009104 (INCB28050), AG490, Tkip, Z3, C7, and TG101209.

In another aspect of any of these methods the Jak2 pathway inhibitor is administered topically. In another aspect of any of these methods the Jak2 pathway inhibitor is administered iv. In another aspect of any of these methods the Jak2 pathway inhibitor is administered p.o.

In another embodiment, the current invention includes a method of modulating INF-induced STAT3 activation by administrating an effective amount of a Type 1 interferon and a STAT3 pathway inhibitor to treat disease. In one aspect of this method, the Type I interferon and STAT3 pathway inhibitor are administered in a single unitary dose.

In one aspect of any of these methods, the STAT3 pathway inhibitor has structural Formula I:

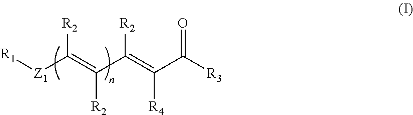

or a pharmaceutically acceptable salt thereof, wherein:

n is 0 or 1;

m is an integer selected from 1, 2, 3, or 4;

R₁ is selected from the group consisting of:

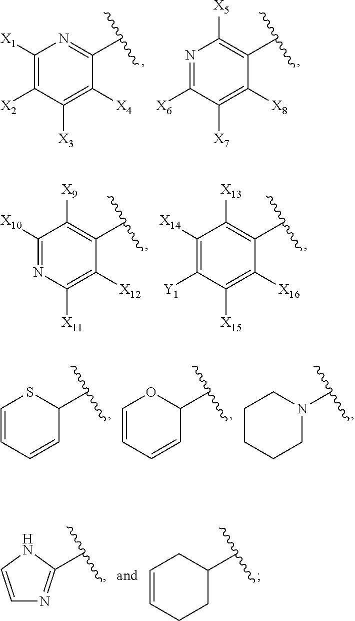

each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;

R₃ is selected from the group consisting of:

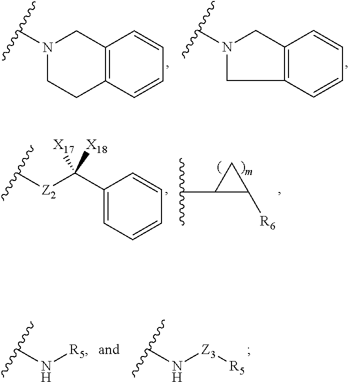

R₄ is selected from the group consisting of cyano, alkylamine, CH₂S-alkyl, alkyl, and CH₂N₃;

R₅ and R₆ are each independently selected from the group consisting of:

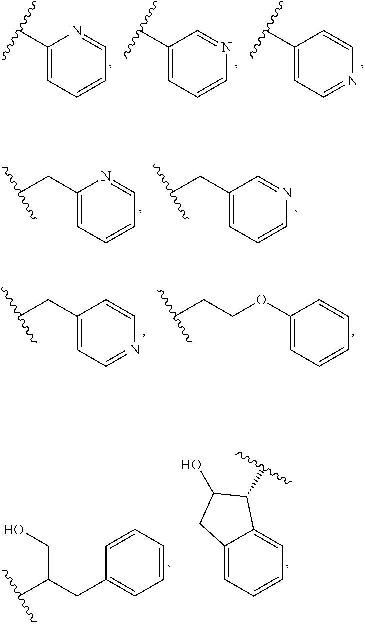

monosaccharide, polysaccharide, monosaccharide derivative, optionally substituted aryl, and optionally substituted arylalkyl;

X₁, X₂, X₃, X₄, X₅, X₆, X₇, X₈, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;

X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;

Y₁ is selected from the group consisting of hydroxyl, halogen, and nitro;

Z₁ is selected from the group consisting of alkyl and a bond;

Z₂ is selected from the group consisting of N H, S, and O; and

Z₃ is alkyl.

In another aspect of this method; R₁ is selected from the group consisting of:

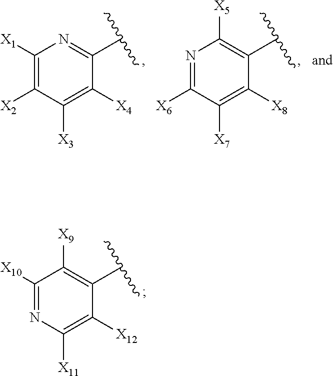

each instance of R₂ is hydrogen;

R₃ is

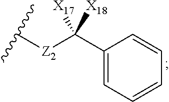

and

Z₂ is NH.

In another aspect of this method; X₁, X₂, X₃, X₄, X₅, X₆, X₇, X₈, X₉, X₁₀, X₁₁, and X₁₂ are each independently selected from the group consisting of hydrogen and halogen; and

X₁₇ and X₁₈ are each independently selected from the group consisting of hydrogen, alkyl, and cycloalkyl.

In another aspect of this method;

R₁ is

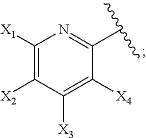

X₁ is halogen; and

X₂, X₃, and X₄ are hydrogen.

In another aspect of this method; one of X₁₇ and X₁₈ is hydrogen;
the other of one of X₁₇ and X₁₈ is selected from the group consisting of hydrogen, methyl, ethyl, and cyclopropyl.

In another aspect of this method; n is 0.

In another aspect of this method; n is 1.

In another aspect of any of these methods, the STAT3 pathway inhibitor is selected from the group of compounds consisting of Examples 1-63.

In another aspect of any of these methods, the STAT3 pathway inhibitor blocks formation of STAT3 homodimers and heterodimers. In another aspect of any of these methods, the STAT3 pathway inhibitor blocks the nuclear translocation of STAT3 and its dimers. In another aspect of any of these methods, the STAT3 pathway inhibitor blocks STAT3 or its dimers or heterodimers DNA binding. In another aspect of any of these methods, the STAT3 inhibitor blocks the phosphorylation of STAT3 at tyrosine 705 and/or serine 727. In another aspect of any of these methods, the STAT3 inhibitor induces secondary processes inactivating pSTAT3. In another aspect of any of these methods, the STAT3 pathway inhibitor decreases levels of pSTAT3.

In another aspect of any of these methods, the STAT3 pathway inhibitor has a structural formula selected from the group consisting of:

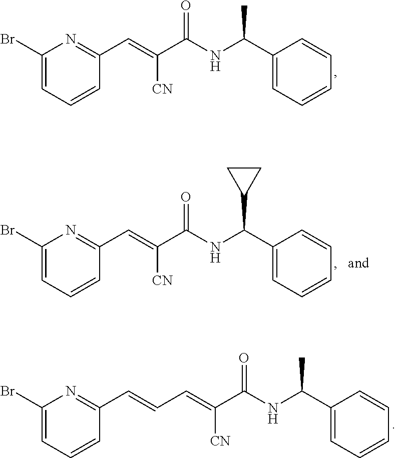 [SMILES: C[C@H](NC(=O)/C(C#N)=C/c1cccc(Br)n1)c1ccccc1 ; N#C/C(=C\c1cccc(Br)n1)C(=O)N[C@H](c1ccccc1)C1CC1 ; C[C@H](NC(=O)/C(C#N)=C/C=C/c1cccc(Br)n1)c1ccccc1]

In another aspect of any of these methods the STAT3 pathway inhibitor is administered topically. In another aspect of any of these methods the STAT3 pathway inhibitor is administered iv. In another aspect of any of these methods the STAT3 pathway inhibitor is administered p.o.

In another embodiment, the current invention includes the use of a STAT3 inhibitor to treat a human patient suffering from a proliferative disease comprising;

a) administering a therapeutic dose of a STAT3 inhibitor to said patient, and

b) administering a type I interferon to said patient.

In another embodiment, the current invention includes the use of a STAT3 inhibitor to reduce the risk or incident of side effects in a human patient

in a regimen which additionally comprises the administration of a Type I interferon, comprising;

a) administering a therapeutic dose of a STAT3 inhibitor to said patient, and

b) administering a type I interferon to said patient.

In another embodiment, the current invention includes the use of a Jak2 inhibitor to reduce the risk or incident of side effects in a human patient

in a regimen which additionally comprises the administration of a Type I interferon, comprising;

a) administering a therapeutic dose of a STAT3 inhibitor to said patient, and

b) administering a type I interferon to said patient.

In one aspect of any of these uses, the STAT3 pathway inhibitor has structural Formula I:

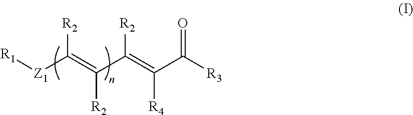

or a pharmaceutically acceptable salt thereof, wherein:

n is 0 or 1;

m is an integer selected from 1, 2, 3, or 4;

R₁ is selected from the group consisting of:

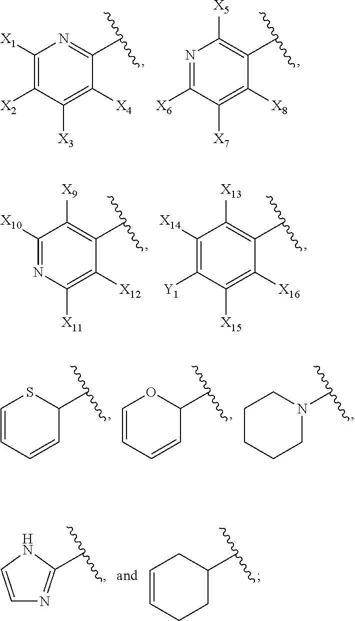

each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;

R₃ is selected from the group consisting of:

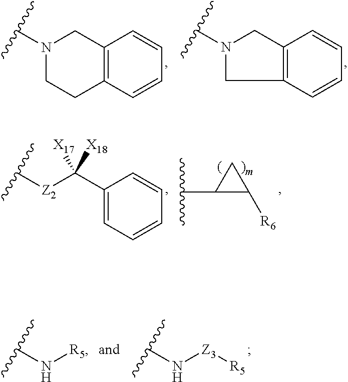

R₄ is selected from the group consisting of cyano, alkylamine, CH₂S-alkyl, alkyl, and CH₂N₃;

R₅ and R₆ are each independently selected from the group consisting of:

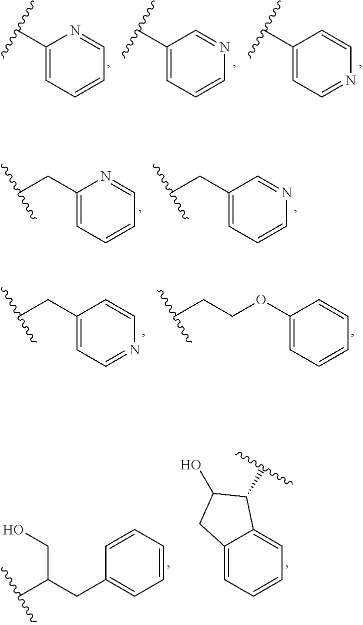

monosaccharide, polysaccharide, monosaccharide derivative, optionally substituted aryl, and optionally substituted arylalkyl;

X₁, X₂, X₃, X₄, X₅, X₆, X₇, X₈, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;

X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;

Y₁ is selected from the group consisting of hydroxyl, halogen, and nitro;

Z₁ is selected from the group consisting of alkyl and a bond;

Z₂ is selected from the group consisting of N H, S, and O; and

Z₃ is alkyl.

In another aspect of any of these uses, R₁ is selected from the group consisting of:

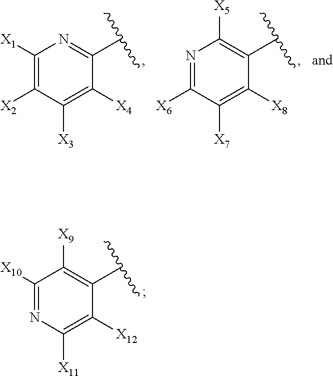

each instance of R₂ is hydrogen;

R₃ is

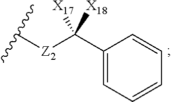

and

Z₂ is NH.

In another aspect of any of these uses,

X₁, X₂, X₃, X₄, X₅, X₆, X₇, X₈, X₉, X₁₀, X₁₁, and X₁₂ are each independently selected from the group consisting of hydrogen and halogen; and

X₁₇ and X₁₈ are each independently selected from the group consisting of hydrogen, alkyl, and cycloalkyl.

In another aspect of any of these uses,

R₁ is

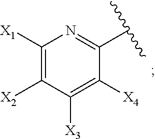

X₁ is halogen; and

X₂, X₃, and X₄ are hydrogen.

In another aspect of any of these uses,
one of X₁₇ and X₁₈ is hydrogen;
the other of one of X₁₇ and X₁₈ is selected from the group consisting of hydrogen, methyl, ethyl, and cyclopropyl.

In another aspect of any of these uses, n is 0.

In another aspect of any of these uses, n is 1.

In another aspect of any of these uses, the STAT3 pathway inhibitor is selected from the group consisting of examples 1-63.

In another aspect of any of these uses, the combination of the STAT3 inhibitor and the Type 1 interferon is characterized by a synergistic response compared to either agent alone.

In another aspect of any of these methods, the proliferative disease is selected from the group consisting of psoriasis, skin cancer, CNS cancer including brain cancer and cancer metastatic to CNS, ovarian cancer, head cancer and neck cancer, prostate cancer, hematological malignancies including leukemia, lymphoma and myeloma, and breast cancer. In one aspect the proliferative disease is skin cancer. In one aspect, the skin cancer is selected from the group consisting squamous cell carcinomas, basal cell cancers, cutaneous T-cell lymphomas, primary cutaneous B cell lymphomas, Dermatofibrosarcoma protuberans, Merkel cell carcinoma, Kaposi's sarcoma, keratoacanthoma, and melanoma.

In another aspect of any of these uses, the proliferative disease is melanoma. In another aspect of any of these methods, the melanoma is CNS melanoma. In another aspect of any of these methods, the patient has Leptomeningeal disease (LMD). In another aspect of any of these uses, the patient has stage III melanoma. In another aspect of any of these methods, the patient has stage IV melanoma.

In another aspect of any of these uses, the side effect of the Type 1 interferon is selected from the group consisting of atypical dermatitis, psoriasis, Crohn's disease, thyroiditis, autoimmune hepatitis, inflammatory bowel disease, and pulmonary fibrosis.

In another aspect of any of these uses, the STAT3 or Jak2 pathway inhibitor is administered topically. In another aspect of any of these methods the STAT3 or Jak2 pathway inhibitor is administered iv. In another aspect of any of these methods the STAT3 or Jak2 pathway inhibitor is administered p.o.

### BRIEF DESCRIPTION OF THE DRAWINGS

A better understanding of the features and advantages of the present invention can be obtained by reference to the following detailed description that sets forth illustrative embodiments, in which the principles of the invention are utilized, and the accompanying drawings of which:

FIG. 1 shows an overview of the Jak/STAT3 signaling pathway.

FIG. 2 depicts WP1066 inhibiting FoxP3 induction in T cells in peripheral blood and downregulates FoxP3 in natural Tregs.

FIGS. 3A, and 3B provide data showing that the novel small molecule, WP1193, inhibits STAT3 activity.

FIG. 4 provides the survival data from C57BL/6J mice treated with WP1193, IFN-α, or both after B16 cells were established in the brain.

FIG. 5 show the CNS and survival data of C57/BL6 mice with intracerebral melanoma treated with WP1193, IFN-α, or both. The figure shows that the C57/BL6 mice died either of LMD or tumor progression depending on the treatment. Both the control and the sub-therapeutically WP1193 group died of progressive LMD. In contrast, in those C57/BL6 mice treated with IFN-α or the combination of IFN-α and WP1193, treatment failure-related deaths were secondary to tumor progression rather than LMD.

FIG. 6 shows the regulation of MHC and NK-activating receptors and their respective ligands by WP1193 and IFN-α. Splenocytes or B16 cells were treated with WP1193, IFN-α, or both, and MHC, the NK-activating receptors and ligands were subsequently analyzed by flow cytometric analysis. The isotype control is shown by the dashed black line and the respective target antigen by a solid black line. FIG. 6A shows B16 cells stained for surface expression of MHC I and II after exposure to WP1193, IFN-α or the combination of WP1193 and IFN-α. FIG. 6B depicts B16 cells stained for surface expression of the NK-activating receptor ligands H60, Rae-1 and CD155 after exposure to WP1193, IFN-α or the combination of WP1193 and IFN-α. FIG. 6C shows NK cells labeled with anti-NK1.1+ antibody from murine splenocytes stained for surface expression of the NK activating receptors NKG2D and KLRD1 after exposure to WP1193, IFN-α or the combination of WP1193 and IFN-α. FIG. 6D shows NK cells labeled with anti-NK1.1+ antibody from murine splenocytes stained for surface expression of the NK activating receptors NKp46, and DNAM-1 after exposure to WP1193, IFN-α or the combination of WP1193 and IFN-α.

FIG. 7 provides data showing IFN-α stimulates tyrosine phosphorylatoin of STAT3 in HH, HuT78 and M JCTCL lines.

FIG. 8A provides data showing WP1220 blocks in a does and time dependent manner constitutive and INFα-induced STAT3 phosphorylation in HH and HuT78 cells. FIG. 8B provides data showing WP1220 blocks in a does and time dependent manner constitutive and INFα-induced STAT3 phosphorylation in MJ CTCL cells.

FIG. 9 provides data showing that WP1220 potently inhibits in vitro growth of HuT78 and HH CTCL lines.

FIG. 10 shows the effect of fixed doses of WP1220 on IFN-induced STAT3 induced phosphorylation in HH CTCL cells.

FIG. 11 shows that the combination of IFNα and WP1066 inhibits ependymoma 58-10F cells growth more potently than either agent alone.

### DETAILED DESCRIPTION

### Definitions

In order that the present disclosure may be more readily understood, certain terms are first defined. Additional definitions are set forth throughout the detailed description. As used herein and in the appended claims, the singular forms “a,” “an,” and “the,” include plural referents unless the context clearly indicates otherwise. Thus, for example, reference to “a molecule” includes one or more of such molecules, “a reagent” includes one or more of such different reagents, reference to “an antibody” includes one or more of such different antibodies, and reference to “the method” includes reference to equivalent steps and methods known to those of ordinary skill in the art that could be modified or substituted for the methods described herein.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl, will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR') group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl(hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The terms “mercaptyl” or “mercaptan” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “monosaccharide” refers to a single basic sugar unit with the general formula Cₙ(H₂O)ₙ, with n ranging from 3 to 8. (e.g. glucose, fructose, galactose, etc.). Monosaccharides may form a glycosidic bond to another group to which they are attached, such as a hydroxyl group or an amino group.

The term “polysaccharide” refers to a polymeric group formed from two or more monosaccharides joined together by glycosidic bonds.

The term “monosaccharide derivative” refers to a monosaccharide that has been chemically modified by addition of one or more protecting groups, such as acetyl groups or diisopropylidene groups (e.g., acetylated galactose, 1,2,3,4-diisopropylideno-D-galactose, etc.).

As used herein, the term “decrease” or the related terms “decreased,” “reduce” or “reduced” refers to a statistically significant decrease. For the avoidance of doubt, the terms generally refer to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease (i.e., the measured parameter is at zero).

As used herein, the term “increase” or the related terms “increased”, “enhance” or “enhanced” refers to a statistically significant increase. For the avoidance of doubt, the terms generally refer to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control value.

As used herein, the term “patient” in the context of the present invention is preferably a mammal. The mammal can be a human, non-human primate, mouse, rat, dog, cat, horse, or cow, but are not limited to these examples. Mammals other than humans can be advantageously used as patients that represent animal models of specific diseases and disorders. A patient can be male or female. A patient can be one who has been previously diagnosed or identified as having cellular degeneration or insufficiency, and optionally has already undergone, or is undergoing, a therapeutic intervention. Preferably the patient is human.

The terms “treating” or “treatment” means to relieve, alleviate, delay, reduce, reverse, improve, manage, or prevent at least one symptom of a condition in a patient. The term “treating” may also mean to arrest, delay the onset (i.e., the period prior to clinical manifestation of a disease), and/or reduce the risk of developing or worsening a condition.

As used herein, the terms “therapeutically effective amount”, “prophylactically effective amount”, or “diagnostically effective amount” is the amount of the active agent, e.g. interferon or STAT3 inhibitor, needed to elicit the desired biological response following administration.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3,4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein.

The publications discussed above are provided solely for their disclosure before the filing date of the present application. Nothing herein is to be construed as an admission that the invention is not entitled to antedate such disclosure by virtue of prior invention.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### Introduction

The activation of the p-STAT3 pathway is useful to prevent anti-tumor immune activity as inflammatory conditions can initate or promote oncogenic transformation. Yu, H. et al, STATs in Cancer Inflammation and Immunity: a Leading Role for STAT3, Nature, Vol. 9, 798-809 (November 2009) (where signalling between STAT3 and NF-kB signalling is noted as interconnected). As discussed by Yu, overexpression or persistent activation of growth factor receptors together with oncogenic mutations in the receptor associated JAK family members may cause some types of cancer. Yu, H. et al, STATs in Cancer Inflammation and Immunity: a Leading Role for STAT3, supra, discussed in detail at pages 799 to 807, incorporated herein by reference. The tyrosine kinase receptors and non-receptor tyrosine kinases such as SRC can be activated by extrinsic pathways such as factors associated with inflammation such as UV radiation or sunlight, chemical carcinogens, infection, stress and cigarette smoke. In turn, the tyrosine kinases induced by both extrinsic and instrinsic pathways phosphorylate STAT3 which in turn forms dimers that translocate to the nucleus where gene expression is directly regulated. In addition to upregulating genes, STAT3 will induce the expression of many cytokines, chemokines and other mediators such as IL-6 and cyclooxygenase 2 that are associated with cancer-promoting inflammation. Most importantly, the receptors for many of the cytokines further active STAT3.

On the other hand, there are systemic interferon IFN-α treatments used to treat cancer currently on the market, such as the FDA approved treatment in melanoma patients.

IFN treatment, however, is not always effective in patients having certain other type of cancer including patients with CNS metastasis or leptomeningeal disease (LMD). But, we have discovered that the efficacy of systemic IFN-α can be significantly enhanced with an inhibitor of the signal transducer and activator of transcription STAT3 in the treatment of established intracerebral syngeneic murine melanoma, including LMD, and other types of cancer.

The Role of STAT3 in Tumorigenesis and Metastasis

Under normal physiological conditions, latent STAT3 activation is dependent on ligand-receptor interaction, primarily under the control of growth factor receptor tyrosine kinases or cytokine and G-protein receptors with associated Jak2. Winston L A, Hunter T, JAK2, Ras, and Raf Are Required For Activation Of Extracellular Signal-Regulated Kinase/Mitogen-Activated Protein Kinase By Growth Hormone, J Biol Chem 1995; 270:30837-30840.

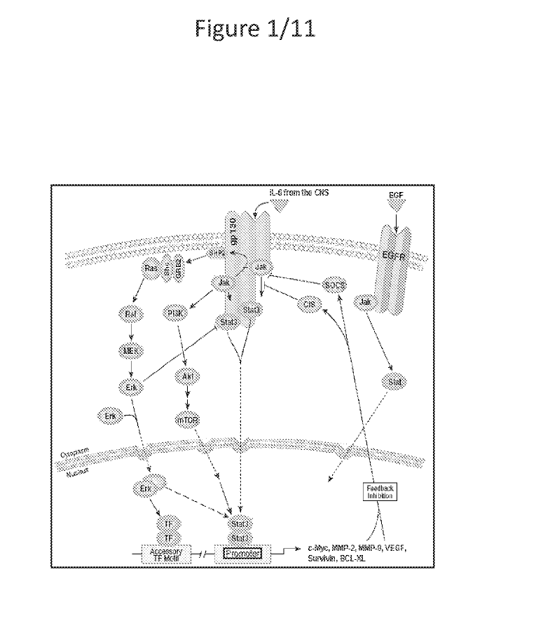

FIG. 1 provides a schematic of the Jak/STAT3 signaling pathway. In the majority of CNS melanoma metastases and primary brain tumors, for example, p-STAT3 is constitutively active. As noted above, the STAT3 pathway, however, can also be induced by cytokines such as IL-6, which is expressed in the CNS under a variety of conditions and by a variety of growth factors. Activation of the STAT3 pathways results in nuclear translocation and subsequent translation of key factors that are responsible for proliferation, resistance to apoptosis, and invasion/metastasis.

Specifically, as noted otherwise herein, the epidermal growth factor receptor (EGFR), interleukin (IL)-6, or IL-4 activate STAT3 by phosphorylation of the tyrosine residue in the transactivation domain of STAT3. Mizoguchi M, Betensky R A, Batchelor T T, Bernay D C, Louis D N, Nutt C L, Activation of STAT3, MAPK, and AKT in Malignant Astrocytic Gliomas: Correlation with EGFR Status, Tumor Grade, And Survival, J Neuropathol Exp Neurol 2006; 65:1181-1188; Rahaman S O, Harbor P C, Chemova O, Barnett G H, Vogelbaum M A, Hague S J, Inhibition Of Constitutively Active Stat3 Suppresses Proliferation And Induces Apoptosis In Glioblastoma Multiforme Cells, Oncogene 2002; 21:8404-8413.

Other non-receptor tyrosine kinases, such as v-src and v-abl, can also activate STAT3 and are among the most frequently activated oncogenic proteins. Upon tyrosine phosphorylation (p-STAT3), dimers of STAT3 are formed, translocate into the nucleus, and induce the expression of a variety of transcriptional factors. Whereas tyrosine phosphorylation of STAT3 regulates dimerization, nuclear translocation, and DNA binding, serine/threonine phosphorylation optimizes transcriptional activity. Turkson J, Ryan D, Kim J, Zhang Y, Chen Z, Haura E, Laudano A, Sebti S, Hamilton A, Jove R, Phosphotyrosyl Peptides Block Stat3-Mediated DNA Binding Activity, Gene Regulation, And Cell Transformation, J Biol Chem 2001; 276:45443-45455.

STAT3, which is frequently overexpressed in many cancers, promotes tumorigenesis by preventing apoptosis (by increasing survivin, BCL-XL, and MCL1 expression) and enhancing proliferation (by increasing c-Myc and cyclin D1/D2 expression), angiogenesis (by increasing VEGF and HIF-1α expression), invasion (by increasing MMP-2 and MMP-9 expression), and metastasis and is a key regulator of immunosuppression. Masamune A, Satoh M, Kikuta K, Suzuki N, Shimosegawa T, Activation of JAK-STAT Pathway Is Required For Platelet-Derived Growth Factor-Induced Proliferation Of Pancreatic Stellate Cells, World J Gastroenterol 2005; 11:3385-3391; Yu H, Jove R, The STATs Of Cancer—New Molecular Targets Come Of Age, Nat Rev Cancer 2004; 4:97-105; Huang S, Regulation Of Metastases By Signal Transducer And Activator Of Transcription 3 Signaling Pathway: Clinical Implications, Clin Cancer Res 2007; 13:1362-1366; Yu H, Kortylewski M, Pardoll D, Crosstalk Between Cancer And Immune Cells: Role Of STAT3 In The Tumour Microenvironment, Nat Rev Immunol 2007; 7:41-51.

In a tumor cell (A), p-STAT3 induces the transcriptional activity of key factors that mediate tumor proliferation and survival (e.g., cyclin D1, BCL-XL), migration and invasion (e.g., MMP-2, MMP-9), and angiogenesis (e.g., VEGF, basic fibroblast growth factor, and HIF-1α). When immune cells became associated with the tumor environment, the STAT3 pathway is activated in these immune cells, resulting in immunosuppression. The precise multi-step processes in the various immune populations have yet to be fully elucidated.

Furthermore, in regulatory T cells (Tregs), the cytokine IL-2 has been shown to activate STAT3, resulting in transcriptional activation of FoxP3, which has been correlated with functional immunosuppressive activity. The activation of STAT3 has also been shown to induce the immunosuppressive cytokine transforming growth factor (TGF)-β and inhibit dendritic cell maturation, the expression of co-stimulatory molecules, and effector T cell proliferation responses. Therefore, blockade of activation of STAT3 and its subsequent nuclear translocation inhibits both tumorigenesis and tumor-mediated immunosuppression.

Investigators have examined activated STAT3 (herein sometimes referred to as “p-STAT3”) expression in malignancies such as gastric, renal, and ovarian cancers; squamous cell and hepatocellular carcinoma; and anaplastic large cell lymphoma and have determined that p-STAT3 expression at tyrosine⁷⁰⁵ correlates with poor prognosis. Other studies have shown that the expression of p-STAT3 correlates with lymph node spread and depth of invasion in colorectal cancer. In contrast, some studies of non-small cell lung cancer and gliomas have shown no relationship between p-STAT3 expression and prognosis. Mizoguchi M, Betensky R A, Batchelor T T, Bernay D C, Louis D N, Nutt C L, Activation Of STAT3, MAPK, And AKT In Malignant Astrocytic Gliomas: Correlation With EGFR Status, Tumor Grade, And Survival, J Neuropathol Exp Neurol 2006; 65:1181-1188; Gong W, Wang L, Yao J, Ajani J A, Wei D, Aldape K, Xie K, Sawaya R, Huang S, Expression Of Activated Signal Transducer And Activator Of Transcription 3 Predicts Expression Of Vascular Endothelial Growth Factor In And Angiogenic Phenotype Of Human Gastric Cancer, Clin Cancer Res 2005; 11:1386-1393; Horiguchi A, Oya M, Shimada T, Uchida A, Marumo K, Murai M, Activation Of Signal Transducer And Activator Of Transcription 3 In Renal Cell Carcinoma: A Study Of Indicence And Its Association With Pathological Features And Clinical Outcome, J Urology 2002; 168:762-765; Meinhold-Heerlein I, Bauerschlag D, Hilpert F, et al., Molecular And Prognostic Distinction Between Serous Ovarian Carcinomas Of Varying Grade And Malignant Potential, Oncogene 2005; 24:1053-1065; Masuda M, Suzui M, Yasumatu R, Constitutive Activation Of Signal Transducers And Activators Of Transcription 3 Correlates With Cyclin D1 Overexpression And May Provide A Novel Prognostic Marker In Head And Neck Squamous Cell Carcinoma, Cancer Res 2002; 62:3351-3355; Shah N G, Trivedi T I, Tankshali R A, STAT3 Expression In Oral Squamous Cell Carcinoma: Association With Clinicopathological Parameters And Survival, Int J Biol Markers 2006; 21:175-183; Yang S, Wang S, Wu C, et al., Altered p-STAT3 (tyr705) Expression Is Associated With Histological Grading And Intratumour Microvessel Density In Hepatocellular Carcinoma, J Clin Pathol 2007; 60:642-648; Khoury J D, Medeiros L J, Rassidakis G, et al., Differential Expression And Clinical Significance Of Tyrosine-Phosphorylated STAT3 In ALK+ And ALK− Anaplastic Large Cell Lymphoma, Clin Cancer Res 2003; 9:3692-3699; Schlette E, Medeiros L J, Goy A, Lai R, Rassidakis G, Surviving Expression Predicts Poorer Prognosis In Anaplastic Large-Cell Lymphoma, J Clin Oncol 2004; 22:1682-1688; Lassmann S, Schuster I, Watch A, et al., STAT3 mRNA And Protein Expression In Colorectal Cancer Effects On STAT3-Inducible Targets Linked To Cell Survival And Proliferation, J Clin Pathol 2007; 60:173-179; Kusaba T, Nakayama T, Yamazumi K, et al., Expression Of p-STAT3 In Human Colorectal Adenocarcinoma And Adenoma; Correlation With Clinicopathological Factors, J Clin Pathol 2005; 58:833-838.

While these studies have addressed p-STAT3 expression at tyrosine⁷⁰⁵, others have shown that p-STAT3 at the serine⁷²⁷ location correlates with the degree of cervical intraepithelial neoplasia. Yang S, Yuan S, Yeh Y, et al., The Role Of p-STAT3 (ser727) Revealed By Its Association With Ki-67 In Cervical Intraepithelial Neoplasia, Gynecologic Oncol 2005; 98:446-452.

A study by Xie et al. confirms the importance of STAT3 in the process of metastasis. Xie T X, Wei D, Liu M, et al., Stat3 Activation Regulates The Expression Of Matrix Metalloproteinase-2 And Tumor Invasion And Metastasis, Oncogene 2004; 23:3550-3560. As shown, only highly metastatic melanoma cell lines overexpress MMP-2 and have elevated levels of p-STAT3. Furthermore, blockade of activated STAT3 by expression of dominant-negative STAT3 significantly suppressed MMP-2 expression and the invasiveness of melanoma cells, inhibited tumor growth, and prevented metastasis in nude mouse model systems. Therefore it is early established that STAT3 activation plays an important role in the dysregulated expression of basic fibroblast growth factor, VEGF, and MMP-2 and confirmed its effects on angiogenesis and its contribution to brain metastasis in melanoma. Subsequent studies of human melanoma cases have demonstrated higher levels of expression of activated STAT3 in brain metastasis specimens than in primary, parenchymal tumors. Xie T X, Huang F J, Aldape K D, et al., Activation Of Stat3 In Human Melanoma Promotes Brain Metastasis, Cancer Res 2006; 66:3188-3196.

Specifically, in 51 parenchymal and 48 brain metastasis specimens obtained from patients with melanoma, only 43% of the former, compared with 81% of the latter, had moderate to strong p-STAT3-positive immunohistochemical staining. Of note, even if a melanoma cell line or specimen does not demonstrate constitutive activation of p-STAT3, IL-6, which can induce the expression of p-STAT3, is expressed in the CNS under a wide variety of conditions, including hypoxia, traumatic and metabolic injury, and inflammation. Tarkowski E, Rosengen L, Blomstrand C, et al., Early Intrathecal Production Of Interleukin-6 Predicts The Size Of Brain Lesion In Stroke, Stroke 1995; 26:1393-1398; Lau L T, Yu A C, Astrocytes Produce And Release Interleukin-1, Interleukin-6, Tumor Necrosis Factor Alpha And Interferon-Gamma Following Traumatic And Metabolic Injury; J Neurotrauma 2001; 18: 351-359; De Simoni M G, Del Bo R, De Luigi A, Simard S, Forloni G, Central Endotoxin Induces Different Patterns Of Interleukin (IL)-1 Beta And IL-6 Messenger Ribonucleic Acid Expression And IL-6 Secretion In The Brain And Periphery, Endocrinology 1995; 136:897-902.

Studies using decoy anti-sense STAT3 oligonucleotides and dominant-negative vectors have provided further convincing evidence that STAT3 is highly relevant to the growth and survival of several tumor types, including melanoma, in vitro and in vivo. Mizoguchi M, Betensky R A, Batchelor T T, Bernay D C, Louis D N, Nutt C L, Activation Of STAT3, MAPK, And AKT In Malignant Astrocytic Gliomas: Correlation With EGFR Status, Tumor Grade, And Survival, J Neuropathol Exp Neurol 2006; 65:1181-1188; Tang G S, Cai J M, Ni J, et al., Effects Of STAT3 Antisense Oligodeoxynucleotides On Apoptosis And Proliferation Of Mouse Melanoma Cell Line B16, Ai Zheng 2006; 25:269-274; Leong P L, Andrews G A, Johnson D E, et al., Targeted Inhibition Of Stat3 With A Decoy Oligonucleotide Abrogates Head And Neck Cancer Cell Growth, Proc Natl Acad Sci USA 2003; 100:4138-4143; Xi S, Gooding W E, Grandis J R, in vivo Antitumor Efficacy Of STAT3 Blockade Using A Transcription Factor Decoy Approach: Implications For Cancer Therapy; Oncogene 2005; 24: 970-979; Chan K S, Sano S, Kiguchi K, et al., Disruption Of STAT3 Reveals A Critical Role In Both The Initiation And The Promotion Stages Of Epithelial Carcinogenesis, J Clin Invest 2004; 114:720-728.

Patients with Cancer have Impaired Immune Responses

Patients with cancer have multiple, redundant mechanisms that contribute to their overall state of immune suppression and act as a barrier to effective immunotherapy. Generalized phenomena of these immune impairments include low peripheral lymphocyte counts, reduced delayed type hypersensitivity reactions to recall antigens, impaired mitogen-induced blastogenic responses by peripheral blood mononuclear cells (PBMCs), and increased regulatory T cells (Tregs). Heimberger A B, Bigner D D, Sampson J J, Biological Principles Of Brain Tumor Immunotherapy, In: Liau L M, Becker D P, Cloughesy T F, Bigner D D, editors. Brain Tumor Immunotherapy. Totowa, N.J.: Humana Press Inc.; 2000; 101-130. Adaptive immune responses are noticeably deficient, with diminished responsiveness of peripheral T cells associated with impaired early transmembrane signaling through the T-cell receptor/CD3 complex. Morford L A, Elliott L H, Carlson S L, Brooks W H, Roszman T L, T Cell Receptor-Mediated Signaling Is Defective In T Cells Obtained From Patients With Primary Intracranial Tumors, J Immunol 1997; 159:4415-4425. In addition, reduced in vitro immunoglobulin synthesis by B cells from the peripheral blood of patients with intracranial tumors appears to be related to diminished T-helper activity. Roszman T, Brooks W, Steele C, Elliott L, Pokeweed Mitogen-Induced Immunoglobulin Secretion By Peripheral Blood Lymphociytes From Patients With Primary Intracranial Tumors. Characterization Of T Helper And B Cell Function, J Immunol 1985; 134:1545-1550.

Many cancers secrete factors, such as prostaglandin E (PGE), IL-10, VEGF, and transforming growth factor (TGF)-β, that are capable of suppressing the cytotoxic responses of T cells against tumor targets, downregulating major histocompatibility complex (MHC) class II expression, suppressing T cell proliferation, and inhibiting the maturation of dendritic cells. Platten M, Wick W, Weller M, Malignant Glioma Biology: Role For TGF-Beta In Growth, Motility, Angiogenesis, And Immune Escape, Microsc Res Tech 2001; 52:401-410; Tada M, de Tribolet N, Recent Advances In Immunobiology Of Brain Tumors, J Neurooncol 1993; 17:261-271; Gabrilovich D, Ishida T, Oyama T, et al., Vascular Endothelial Growth Factor Inhibits The Development Of Dendritic Cells And Dramatically Affects The Differentiation Of Multiple Hematopoietic Lineages In Vivo, Blood 1998; 92:4150-4166.

Targeting a single immunosuppressive cytokine is not likely to be efficacious because tumors usually express a variety of immunosuppressive cytokines; the blockade of any one cytokine would not be expected to significantly impact the overall immunosuppressive milieu. Surgical resection of a tumor can reduce the influence of these factors, however.

The absence or low expression of CD80 gives an immune escape advantage to cancer cells because CD28-mediated co-stimulatory signals are essential for the differentiation of functional tumor-specific CD8+ T-effector cells. Tirapu I, Huarte E, Guiducci C, Arina A, et al., Low Surface Expression Of B7-1 (CD80) Is An Immunoescape Mechanism Of Colon Carcinoma, Cancer Res 2006; 66:2442-2450; Voigt H, Schrama D, Eggert A O, et al., CD28-Mediated Costimulation Impacts On The Differentiation Of DC Vaccination-Induced T Cell Responses, Clin Exp Immunol 2006; 143:93-102. In the brain, microglia are macrophage-like CNS antigen-presenting cells (APC) that are presumably capable of innate immune functions and antigen presentation. Aloisi F, Immune Function Of Microglia, Glia 2001; 36:165-179. Since T cell activation requires signals through both MHC and co-stimulatory molecules, the expression of MHC alone on microglia would not activate a T cell response and could result in T cell anergy. Yi-qun Z, Lorre K, de Boer M, Ceuppens J L, B7-Blocking Agents, Alone Or In Combination With Cyclosporin A, Induce Antigen-Specific Anergy Of Human Memory T Cells, J Immunol 1997; 158:4734-4740. Microglia expressing low levels of co-stimulatory molecules have been shown to be unable to activate either naïve or primed T cells and to induce T cell anergy. Matyszak M K, Denis-Donini S, Citterio S, Longhi R, Granucci F, Ricciardi-Castagnoli P, Microglia Induce Myelin Basic Protein-Specific T Cell Anergy Or T Cell Activation, According To Their State Of Activation, Eur J Immunol 1999; 29:3063-3076.

Microglia isolated from human melanoma metastases to the CNS and primary brain tumors express MHC class II molecules but lack expression of the co-stimulatory molecules CD86, CD80, and CD40, which are critical for T cell activation. Hussain S F, Yang D, Suki D, Aldape K, Grimm E, Heimberger A B, The Role Of Human Glioma-Infiltrating Microglia/Macrophages In Mediating Antitumor Immune Responses, Neuro Oncol 2006; 8:261-279. In tumor-bearing animals, repeated stimulation of the T cells is necessary in order to generate tumor responses indicating that failure of the APC to provide appropriate stimulation is a central component of immune failure. Gabrilovich D I, Ciemik I F, Carbone D P, Dendritic Cells In Antitumor Immune Responses. I. Defective Antigen Presentation In Tumor-Bearing Hosts, Cell Immunol 1996; 170:101-110.

In addition to the numerous immunosuppressive mechanisms already discussed, recent studies have demonstrated that Tregs are responsible for the inhibition of tumor-reactive effector T cells, and elimination of Tregs by any of several different strategies successfully enhances antitumor immunity. Attia P, Maker A V, Haworth L R, Rogers-Freezer L, Rosenberg S A, Inability Of A Fusion Protein Of IL-2 And Diphtheria Toxin (Denileukin Diftitox, DAB389IL-2, ONTAK) To Eliminate Regulatory T Lymphocytes In Patients With Melanoma, J Immunother 2005; 28:582-592; Berd D, Mastrangelo M J, Effect Of Low Dose Cyclophosphamide On The Immune System Of Cancer Patients: Reduction Of T-Suppressor Function Without Depletion Of The CD8+ Subset, Cancer Res 1987; 47:3317-3321; Fecci P E, Ochiai H, Mitchell D A, et al., Systemic CTLA-4 Blockade Ameliorates Glioma-Induced Changes To The CD4+ T Cell Compartment Without Affecting Regulatory T-Cell Function, Clin Cancer Res 2007; 13:2158-2167; Fecci P E, Sweeney A E, Grossi P M, et al., Systemic Anti-CD25 Monoclonal Antibody Administration Safely Enhances Immunity In Murine Glioma Without Eliminating Regulatory T Cells, Clin Cancer Res 2006; 12:4294-4305; Su Y B, Sohn S, Krown S E, et al., Selective CD4+ Lymphopenia In Melanoma Patients Treated With Temozolomide: A Toxicity With Therapeutic Implications, J Clin Oncol 2004; 22: 610-616.

Similarly, CD4+CD25+FoxP3+ Treg-mediated suppression has also been demonstrated in several human cancers with increased numbers of Tregs present in both human gliomas and metastatic cancers to the CNS. Curiel T J, Coukos G, Zou L, et al., Specific Recruitment Of Regulatory T Cells In Ovarian Carcinoma Fosters Immune Privilege And Predicts Reduced Survival, Nat Med 2004; 10: 942-949; Fecci P E, Mitchell D A, Whitesides J F, et al., Increased Regulatory T-Cell Fraction Amidst A Diminished CD4 Compartment Explains Cellular Immune Defects In Patients With Malignant Glioma Cancer Res 2006; 66:3294-3302; Fontenot J D, Rudensky A Y, A Well Adapted Regulatory Contrivance: Regulatory T Cell Development and The Forkhead Family Transcription Factor Foxp3, Nat Immunol 2005; 6:331-337; Liyanage U K, Moore T T, Joo H G, et al., Prevalence Of Regulatory T Cells Is Increased In Peripheral Blood And Tumor Microenvironment Of Patients With Pancreas Or Breast Adenocarcinoma, J Immunol 2002; 169:2756-2761; Heimberger A B, Reina-Ortiz C, Yang D S, et al., Incidence And Prognostic Impact Of FoxP3+ Regulatory T Cells In Human Gliomas, Clin Cancer Res (In Press); Kong L Y, Abou-Ghazal M K, Wei J, et al., A Novel Inhibitor Of STAT3 Activation Is Efficacious Against Established Central Nervous System Melanoma And Inhibits Regulatory T Cells, Clin Cancer Res (Submitted).

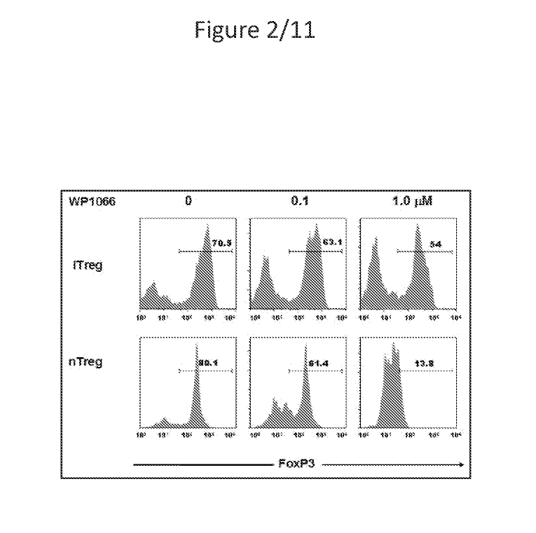

Collectively, these data indicate that Tregs can not only inhibit initial systemic immune activation but also prevent effector responses in the tumor microenvironment. A key regulator of all of these immune-suppressive mechanisms is STAT3. See, FIG. 2.

STAT3 Modulates Immunosuppression

While STAT3 may be a potent factor that regulates immunosuppression by preventing the maturation of dendritic cells and inhibiting the proliferation and activation of immune effector populations, the tumor microenvironment has multiple mechanisms to down-modulate immune responses, as described herein. Overall, to date, immunotherapy for solid tumors has not resulted in objective efficacy. Rosenberg S A, Yang J C, Restifo N P, Cancer Immunotherapy: Moving Beyond Current Vaccines, Nat Med 2004; 10: 909-915. Although CNS tumors are recognized by the immune system, this is not sufficient to suppress or eradicate them. Primed CD8+ cytotoxic T cells gain CNS access; however, the lack of tumor eradication indicates that the T cells are functionally impaired within the tumors. Heimberger A B, Reina-Ortiz C, Yang D S, et al., Incidence And Prognostic Impact Of FoxP3+ Regulatory T Cells In Human Gliomas, Clin Cancer Res (In Press); Calzascia T, Masson F, Di Berardino-Besson W, et al., Homing Phenotypes Of Tumor-Specific CD8 T Cells Are Predetermined At The Tumor Site By Crosspresenting APCs, Immunity 2005; 22:175-184; Hickey W F, Hsu B L, Kimura H, T-Lymphocyte Entry Into The Central Nervous System, J Neurosci Res 1991; 28:254-260. This is confirmed by isolating effector T cells in CNS metastasis and primary gliomas as and found that they were phenotypically CD8⁺CD25. The blood-brain barrier is not functional within tumors, as evidenced by the extravasation of gadolinium on magnetic resonance imaging of the CNS to visualize malignant brain tumors and metastasis.

The p-STAT3-expressing tumor, via undefined mechanisms, subsequently induces STAT3 activity in tumor-associated immune cells. Yu H, Kortylewski M, Pardoll D, Crosstalk Between Cancer And Immune Cells: Role Of STAT3 In The Tumour Microenvironment, Nat Rev Immunol 2007; 7:41-51; Kortylewski M, Yu H, Stat3 As A Potential Target For Cancer Immunotherapy; J Immunother (1997) 2007; 30:131-139. This induced p-STAT3 expression in effector immune cells causes anti-inflammatory responses by suppressing macrophage activation and limiting inflammatory responses. Lang R, Patel D, Morris J, Rutschman R, Murray P, Shaping Gene Expression In Activated And Resting Primary Macrophages By IL-10, J Immunol 2002; 169:2253-2263; O'Farrell A M, Liu Y W, Moore K W, Mui A L. IL-10 Inhibits Macrophage Activation And Proliferation By Distinct Signaling Mechanisms: Evidence For STAT3 Dependent And-Independent Pathways, EMBO J. 1998; 17: 1006-1018; Takeda K, Clausen B, Kaisho T, et al., Enhanced Th1 Activity And Development Of Chronic Enterocolitis In Mice Devoid Of Stat3 In Macrophages And Neutrophils, Immunity 1999; 10: 39-49; Lin T, Bost K, STAT3 Activation In Macrophages Following Infection With Salmonella, Biochem Biophys Res Commun 2004; 321: 828-834.

STAT3 activity within natural killer (NK) cells and neutrophils directly reduces their cytotoxicity, whereas STAT3 activity in dendritic cells reduces the expression of MHC II, CD80, CD86, and IL-12 in these cells, rendering them unable to stimulate T cells and generate antitumor immunity. In contrast, the induced p-STAT3 expression in the immune inhibitor Treg population likely renders them functionally active. IL-2 has been shown to regulate FoxP3 expression in human CD4+CD25+Tregs by inducing STAT3 binding of the first intron of the FoxP3 gene. Zorn E, Nelson E A, Mohseni M, et al., IL-2 Regulates FOXP3 Expression In Human CD4+CD25+ Regulatory T Cells Through A STAT-Dependent Mechanism And Induces The Expansion Of These Cells In Vivo, Blood 2006; 108:1571-1579. Suppressor of cytokine signaling-3 has been shown to be an inhibitor of STAT3 signaling and transcriptional activity but is deficient in Tregs. Yoshimura A, Naka T, Kubo M, SOCS Proteins, Cytokine Signalling And Immune Regulation, Nat Rev Immunol 2007; 7:454-465; Starr R, Willson T A, Viney E M, et al., A Family Of Cytokine-Inducible Inhibitors Of Signalling, Nature 1997; 387:917-921; Qin H, Roberts K L, Niyongere S A, Cong Y, Elson C O, Benveniste E N, Molecular Mechanism Of Lipopolysaccharide-Induced SOCS-3 Gene Expression In Macrophages And Microglia, J Immunol 2007; 179: 5966-5976; Pillemer B B, Xu H, Oriss T B, Qi Z, Ray A, Deficient SOCS3 Expression In CD4+CD25+FoxP3+ Regulatory T Cells And SOCS3-Mediated Suppression Of Treg Function, Eur J Immunol 2007; 37: 2082-2089.

Certain agents designed to block p-STAT3 should inhibit the induction of Tregs while stimulating pro-inflammatory effector responses. A study by Kortylewski et al. provides the definitive evidence of the role of the immune system in tumor clearance with p-STAT3 blockade. Kortylewski M, Kujawski M, Wang T, et al., Inhibiting Stat3 Signaling In The Hematopoietic System Elicits Multicomponent Antitumor Immunity, Nat Med 2005; 11: 1314-1321. They showed that the ablation of STAT3 in only the hematopoietic cells in mice resulted in marked enhancement of activated and functional T cells, NK cells, and dendritic cells in tumor-bearing mice. This ablation of STAT3 in only the hematopoietic cells resulted in marked antitumor effects in vivo, indicating that STAT3 expression in immune cells restrains antitumor immune eradication. Id.

As depicted in FIG. 1 and noted above, the activation of the STAT3 pathways results in nuclear translocation and subsequent translation of key factors that are responsible for proliferation, resistance to apoptosis, and invasion/metastasis. For example, in the majority of CNS melanoma metastases and primary brain tumors, p-STAT3 is constitutively active. The STAT3 pathway can also be induced by cytokines such as IL-6, which can be expressed in the CNS under a variety of conditions and by a variety of growth factors. In a tumor cell, p-STAT3 (activated STAT3) induces the transcriptional activity of key factors that mediate tumor proliferation and survival (e.g., cyclin D1, BCL-XL), migration and invasion (e.g., MMP-2, MMP-9), and angiogenesis (e.g., VEGF, basic fibroblast growth factor, and HIF-1α). When immune cells became associated with the tumor environment, the STAT3 pathway is activated in these immune cells, resulting in immunosuppression.

Furthermore, while the precise multi-step processes in the various immune populations have yet to be fully elucidated. in regulatory T cells (Tregs), the cytokine IL-2 has been shown to activate STAT3, resulting in transcriptional activation of FoxP3, which has been correlated with functional immunosuppressive activity. The activation of p-STAT3 has also been shown to induce the immunosuppressive cytokine transforming growth factor (TGF)-β and inhibit dendritic cell maturation, the expression of co-stimulatory molecules, and effector T cell proliferation responses.

STAT's (STAT3 and STAT5 particularly) are up regulated in many cancers including glioblastoma, head and neck cancer head, prostate cancer, leukemias and breast cancer. A constitutively active form of STAT3 is oncogenic, though these mutations have not been identified in human cancer as yet. STAT3 activation is associated with a number of inflammatory diseases of the skin, gut, respiratory system and brain; such as psoriasis, Crohn's disease, inflammatory bowel disease (IBD), pulmonary fibrosis and acute lung injury, as well as multiple sclerosis (M.S.). STAT3 is also critical for leptin signaling and its mutation leads to obesity in mice.

The blockade of activation of STAT3 and its subsequent nuclear translocation inhibits both tumorigenesis and tumor-mediated immunosuppression. For example, as shown in FIG. 2, WP1066 inhibiting FoxP3 induction in T cells in peripheral blood and down-regulates FoxP3 in natural Tregs. CD4⁺CD25⁻CD62L^{hi} naïve T cells from C57BL/6J mice were stimulated by plate-bound anti-CD3 (2 μg/ml) and soluble anti-CD28 (2 μg/ml) in the presence of TGF-β1 (1 ng/ml) and hIL-2 (200 U/ml), with 0, 0.1, and 1.0 μM WP1066 for inducible Tregs (iTreg) differentiation. CD4⁺CD25⁺ T cells (natural Tregs, nTreg) were stimulated by plate-bound anti-CD3 (2 μg/ml) and soluble anti-CD28 (2 μg/ml) in the presence of hIL-2 (200 U/ml), with 0, 0.1, and 1.0 μM WP1066. Ninety-six hours after stimulation, the cells were analyzed for intracellular FoxP3 expression by flow cytometry. The percentage of FoxP3 expressing T cells is shown.

The especially high selectivity of IFN-α and IFN-β toward STAT3 activation was observed in CTCL tumors where none of the other usually potent activators of STAT3, including IL-6, were able to induce STAT3 phosphorylation. As a result, inhibiting IFN-induced STAT3 activation will potentiate the activity of IFNs. For example, we demonstrated that the combination of IFN-α and p-STAT3 blockade can exert efficacy against intracerebral established CNS melanoma. Patients with CNS melanoma, especially those with LMD, are typically refractory to currently available standard therapies and our preclinical data would suggest that this combination might have clinical utility. This is notable considering that immunotherapeutic approaches for melanoma have been disappointing. Rosenberg S A, Yang J C, Restifo N P, Cancer Immunotherapy: Moving Beyond Current Vaccines, Nat Med 2004; 10:909-15. The cytokine IFN-α is currently FDA-approved for patients with stage III melanoma; however, only 15% of melanoma patients have an objective response. Agarwala S S, Kirkwood J M, Interferons in Melanoma, Curr Opin Oncol 1996; 8:167-74.

As described herein and more specifically below, in our experimental model system of CNS melanoma, IFN-α induced a modest therapeutic response. But when IFN-α was used in combination with WP1193, marked therapeutic efficacy was seen and more specifically, the combination markedly inhibited the development of LMD. Previous immune therapeutic approaches to treat melanoma LMD included the intrathecal administration of IL-2. Herrlinger U, Buchholz R, Jachimczak P, Schabet M., Intrathecal Treatment of C6 Glioma Leptomeningeal Metastasis in Wistar Rats With Interleukin-2, J Neurooncol 1996; 27:193-203; List J, Moser R P, Steuer M, Loudon W G, Blacklock J B, Grimm E A, Cytokine Responses To Intraventricular Injection Of Interleukin 2 Into Patients With Leptomeningeal Carcinomatosis: Rapid Induction Of Tumor Necrosis Factor A, Interleukin 1β, Interleukin 6, Γ-Interferon, And Soluble Interleukin 2 Receptor (Mr 55,000 Protein), Cancer Res. 1992; 52:1123-8; Obbens E A, Feun L G, Leavens M E, Savaraj N, Stewart D J, Gutterman J U, Phase I Clinical Trial Of Intralesional Or Intraventricular Leukocyte Interferon For Intracranial Malignancies, J Neurooncol 1985; 3:61-7.

IL-2 enhances NK cell activity, activates cytotoxic T cells, stimulates IFN-γ release and activates macrophages and IFN-α has direct anti-proliferative effects on tumor cells, activates NK cells and cytotoxic T cells, and enhances antigen presentation and MHC expression. Clinical trials of IL-2 in melanoma patients with LMD demonstrated a high rate of tumor clearance from the CSF; however treatment resulted in meningeal irritation, fever, brain edema, seizures, stupor and one death. Moser R. P., et al., Biologic Therapy For Brain Tumors, Cancer Bull 1991; 43:117-26; Papadopoulos N. E., et al., Intrathecal Use Of Recombinant Interleukin-2 (Ril-2) In The Treatment Of Leptomeningeal Disease (LMD) From Metastatic Melanoma, Proc Annu Meet Am Soc Clin Oncol 1995; 14; Heimans J. J., et al., Treatment Of Leptomeningeal Carcinomatosis With Continuous Intraventricular Infusion Of Recombinant Interleukin-2, Surg Neurol 1991; 35:244-7.

Similarly, in clinical trials IFN-α also demonstrated clearance of malignancy within the CSF; however treatment was confounded by profound neuro-toxicity. Obbens E. A., et al., Phase I Clinical Trial Of Intralesional Or Intraventricular Leukocyte Interferon For Intracranial Malignancies, J Neurooncol 1985; 3:61-7; Meyers C. A., et al., Neurotoxicity Of Intraventricularly Administered Alpha-Interferon For Leptomeningeal Disease, Cancer 1991; 68:88-92.

### Combining IFN-α and WP1193 Enhanced Tumor Cytotoxicity Mediated by Both the NK and CD8+ T Cell Populations

To determine whether the enhancement of NK-mediated cytotoxic function was related to up-regulation of NK-activating receptors or ligands by treatment with the combination of IFN-α and WP1193, we assessed the expression levels on NK cells and on B16, respectively. The B16 cells expressed the MHC I, Rae1, H60 and CD155, indicating that they would be capable of triggering NK cytotoxic responses, resulting in tumor clearance similar to findings in a previous report, but treatment did not alter the expression of the ligands. Diefenbach A, Jensen E R, Jamieson A M, & Raulet D H, Rae1 And H60 Ligands Of The NKG2D Receptor Stimulate Tumour Immunity, Nature 2001; 413:165-71.

Furthermore, we did not find changes in the NK-activating receptor expression levels of NKG2D, KLRD1, NKp46 or DNAM-1. It is possible that there was a transient increase in the NK-activating receptors that was not identified under the current experimental conditions, but most likely the enhancement of both the CD8+ and NK cytotoxic activity with the IFN-α and WP1193 therapy was related to the enhancement of pro-inflammatory cytokines that enhanced function. To investigate this we evaluated the elaboration of pro-inflammatory cytokines and found that combinational therapy markedly induced IFN-γ in vivo. IFN-γ has been shown to markedly promote both NK and cytotoxic T cell activity, to increase the expression of MHC, and enhance antigen expression. Palmer K J, Harries M, Gore M E, Collins M K, Interferon-Alpha (IFN-Alpha) Stimulates Anti-Melanoma Cytotoxic T Lymphocyte (CTL) Generation In Mixed Lymphocyte Tumour Cultures (MLTC), Clin Exp Immunol 2000; 119:412-8; Carballido J A, Molto L M, Manzano L, Olivier C, Salmeron O J, Alvarez de Mon M, Interferon-Alpha-2b Enhances The Natural Killer Activity Of Patients With Transitional Cell Carcinoma Of The Bladder, Cancer 1993; 72:1743-8; Kaser A, Enrich B, Ludwiczek O, Vogel W, Tilg H, Interferon-Alpha (IFN-A) Enhances Cytotoxicity In Healthy Volunteers And Chronic Hepatitis C Infection Mainly By The Perforin Pathway, Clin Exp Immunol 1999; 118:71-7; Keir M E, Stoddart C A, Linquist-Stepps V, Moreno M E, McCune J M, IFN-A Secretion By Type 2 Predendritic Cells Up-Regulates MHC Class I In The HIV-1-Infected Thymus, J Immunol 2002; 168:325-31; Greiner J W, Fisher P B, Pestka S, Schlom J, Differential Effects Of Recombinant Human Leukocyte Interferons On Cell Surface Antigen Expression, Cancer Res 1986; 46:4984-90.

The enhancement of some immune stimulatory cytokines such as TNF-α and IFN-γ can also exert direct cytotoxic tumor effects and both of these were induced with IFN-α and WP1193 in vivo; thus, it is possible that the TNF-α and IFN-γ also exerted direct effects on the intracerebral B16 and could be participating in the observed in vivo efficacy.

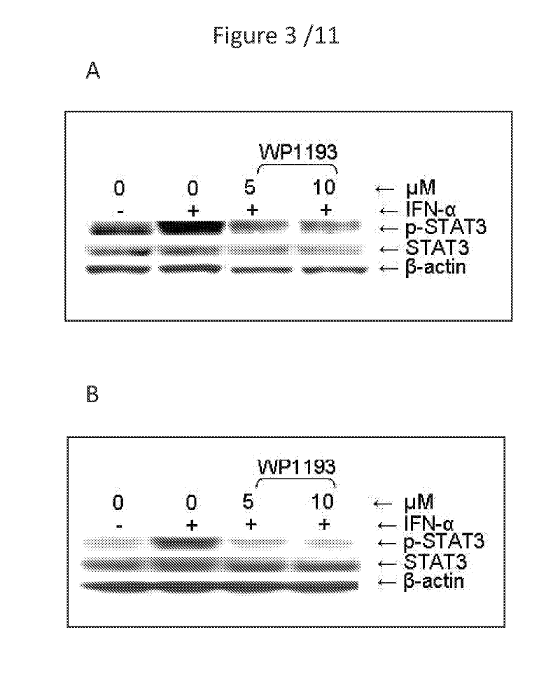

As shown in FIGS. 3A, and 3B, WP1193 inhibits the phosphorylation of p-STAT3 in both B16 cells (FIG. 3A) and in splenocytes (FIG. 3B). B16 cells and splenocytes isolated from C57BL/6J mice and were incubated with either the medium, medium supplemented with titrated WP1193, medium supplemented with IFN-α, or medium supplemented with both IFN-α and WP1193. After 2 hours (splenocytes) or 4 hours (B16 cells), cells were lysed, electrophoretically fractionated in 8% SDS-polyacrylamide gels, transferred to nitrocellulose membranes, and immunoblotted with antibodies to p-STAT3, total STAT3 and β-actin. Semi-quantitative densitometry was used to determine the relative levels of p-STAT3 to STAT3 and β-actin.

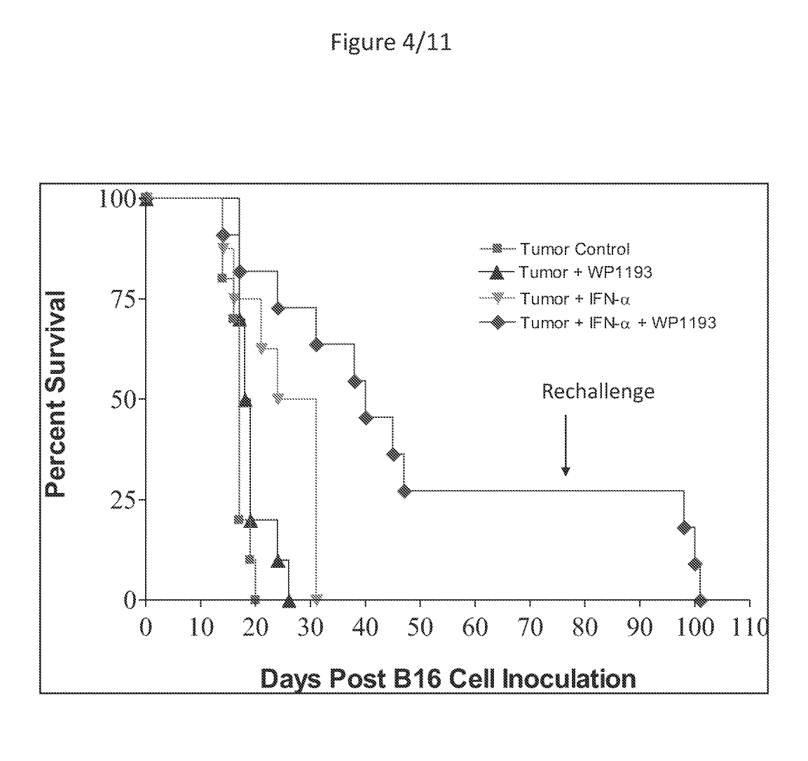

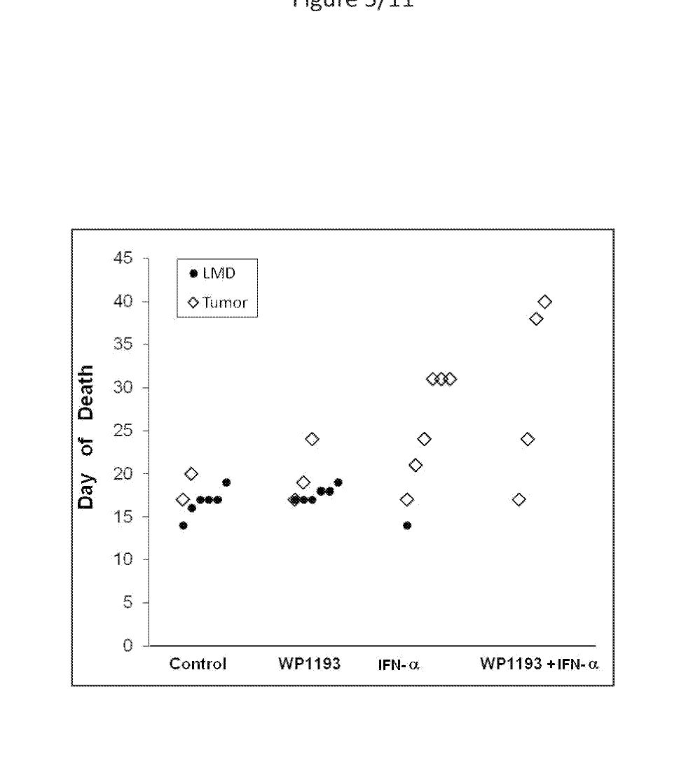

Likewise, FIG. 4 provides the survival data from C57BL/6J mice treated with WP1193, IFN-α, or both after B16 cells were established in the brain. Median overall survival for mice with intracerebral tumors without further intervention (n=11) was 17 days. C57BL/6J mice with established intracerebral B16 cells treated with a sub-therapeutic dose of WP1193 via oral gavage (n=12) showed a 9% increase in their median survival time to 18.5 days (P<0.04 compared with control). In mice with established tumor treated with IFN-α alone (n=8), there was a 62% increased in median survival to 27.5 days (P<0.01 compared with control). In those mice with established tumors treated with the combination of IFN-α and WP1193 (n=11), there was a 135% increase in median survival to 40 days that was significantly longer compared with IFN-α alone (P<0.02). In mice that survived long-term subsequent re-challenge by injection of B16 cells into the contralateral hemisphere indicated that minimal immunological memory was induced. This experiment was repeated in its entirety with similar results. Therefore, the combination a STAT3 inhibitor such as WP1193 together with IFN-α enhances both NK and CD8+ cytotoxicity by enhancing pro-inflammatory cytokines and can be a treatment modality for melanoma patients with CNS disease who currently have very few therapeutic options available and who are typically excluded from clinical trials.

We have further shown that the p-STAT3 inhibitors inhibit Tregs in murine models of melanoma and from melanoma patients. Kong L Y, et al, A Novel Inhibitor Of Signal Transducers And Activators Of Transcription 3 Activation Is Efficacious Against Established Central Nervous System Melanoma And Inhibits Regulatory T Cells, Clin Cancer Res 2008; 14:5759-68; Kong L-K, Wei J, Sharma A K, Barr J, Abou-Ghazal M K, Fokt I, Weinberg J, Rao G, Grimm E, Priebe W, Heimberger A B, A Novel Phosphorylated STAT3 Inhibitor Enhances T Cell Cytotoxicity Against Melanoma Through Inhibition Of Regulatory T Cells, Cancer Immunol Immunother 2008 58:1023-32.

IFN-α has been shown to augment IL-10 production and IFN-α-treated dendritic cells induce IL-10-producing regulatory T cells. Ito T, Amakawa R, Inaba M, Ikehara S, Inaba K, Fukuhara S, Differential Regulation Of Human Blood Dendritic Cell Subsets By Ifns, J Immunol 2001; 166:2961-9. Additionally, in studies of human melanoma patients treated with high-dose IFN-α, Wang et al., demonstrated an enhancement of Tregs in the lymph nodes but the Treg population was not analyzed in the bone marrow and blood. Wang W, Edington H D, Rao U N, Jukic D M, Radfar A, Wang H, Kirkwood J M, Effects Of High-Dose Ifnα2b On Regional Lymph Node Metastases Of Human Melanoma: Modulation Of STAT5, FOXP3, And IL-17, Clin Cancer Res 2008; 14:8314-20.

Furthermore, we found that IFN-α induces the immune suppressive p-STAT3 and others have shown that p-STAT3 is a promoter of FoxP3 expression in Tregs. Thus, we hypothesized that the p-STAT3 inhibitors would enhance the therapeutic efficacy of IFN-α by inhibiting the induced Tregs. Although there is inhibition of the number of Tregs in both the bone marrow and blood with IFN-α and WP1193, there was no additive effect on inhibiting the number of Tregs. Interestingly, IFN-α demonstrated inhibition of the number of Tregs in bone marrow and the peripheral blood and slight enhancement of the numbers of Tregs in the lymph nodes and so a paradox arises as to the mechanism of IFN-α in inhibiting Tregs that we observed in vivo. Within a few days after hydrodynamic gene transfer of IFN-γ, the total bone marrow cellularity drops with the CD4 T cell population being the most affected which is consistent with other reports of IFN-α.

Within the CD4 fraction, the FoxP3+ Tregs numbers are even more suppressed compared with non-Treg CD4+ T cells, thus the reason why in the IFN-γ treatment group the Treg numbers were most dramatically inhibited within the bone marrow. Using sorted Tregs from a FoxP3-GFP reporter mouse from IFN-α-treated or control mice we did not see a decrease in the suppressive activity (data not shown). Thus, IFN-α inhibits the relative number of Tregs but not their suppressive activity, whereas WP1193 only modestly inhibits the number of Tregs in the bone marrow compared with IFN-α.

IFN-α is also being used in non-cancer related therapies and during such therapies IFN-α was documented as being the major factor associated with inducing outbursts of psoriasis. We have discovered that IFN-α, IFN-β and also IL-6 will potently induce STAT3 activation in a keratinocytes. STAT3 was independently shown to an important factor in psoriasis. Therefore the direct or indirect inhibitors of STAT3 is useful to block IFN-α therapy induced psoriasis.

### I. Overview of Methods of Treatment

The invention is based, in part, on the discovery that IFN-α and IFN-β (also noted herein sometimes as IFNα and IFNβ) can selectively and potently activate STAT3. As a consequence, induced tumor cell proliferation and survival as well as other downstream signaling events leading to increased angiogenesis and tumor immunotolerance occur. This activation, in part, undermines the antitumor effects of IFNs. Therefore the invention provides improved methods of treating cancer, and reducing the side effects of INF therapy.

Thus in one aspect, the present invention includes a method of treating a proliferative disease comprising the step of administering to a patient a therapeutically effective amount of Type 1 interferon in combination with a STAT3 pathway inhibitor.

In another aspect, the present invention includes a method of potentiating the activity of Type 1 interferon for treatment of a proliferative disease comprising the step of administering to a patient a therapeutically effective amount of Type 1 interferon in combination with a STAT3 pathway inhibitor.

In another aspect, the present invention includes a method of modulating anti-viral therapy with a type I interferon, comprising the step of administering to a patient a therapeutically effective amount of Type 1 interferon in combination with a Jak2 or STAT3 pathway inhibitor, wherein the STAT3 pathway inhibitor, or Jak2 inhibitor, reduces the severity of at least one side effect of the Type 1 interferon.

In another aspect, the present invention includes a method of modulating IFN-induced STAT3 activation during treatment for viral hepatitis comprising the step of administering to a patient a therapeutically effective amount of Type 1 interferon in combination with a STAT3 pathway inhibitor.

In this context “administered in combination” means: (1) part of the same unitary dosage form; (2) administration separately, but as part of the same therapeutic treatment program or regimen, typically but not necessarily, on the same day.

In one aspect of any of these methods, the combination of the STAT3 inhibitor and the Type 1 interferon is characterized by a synergistic biological response compared to either agent alone. By “synergistic biological response” in this context is meant that the combination therapy provides for a greater than additive effect (after subtraction of appropriate control values) on at least one biological outcome of the treatment. For example, such as median overall survival time in mice after implantation of tumor cells, overall survival rates after a certain time, % of cell killing, the rate of tumor growth, invasion, or tumor size, etc.

In different aspects, the synergistic response of the combination therapy may be greater than each individual monotherapy response by at least about 5%, at least about 10%, at least about 30%, at least about 40%, at least about 50%, at least about 60%, at least about 80%, at least about 90%, or at least about 100%, or greater.

In another aspect of any of these methods, the STAT3 pathway inhibitor potentiates the activity of the Type I interferon treatment. By “potentiates” in this context is meant that the combination therapy provides for a greater effect than interferon monotherapy alone (after subtraction of appropriate control values) on at least one biological outcome of the treatment. For example, such as median overall survival time in mice after implantation of tumor cells, overall survival rates after a certain time, % of cell killing, the rate of tumor growth, invasion, or tumor size, etc.

In different aspects, the combination therapy may potentiate the IFN monotherapy response by at least about 5%, at least about 10%, at least about 30%, at least about 40%, at least about 50%, at least about 60%, at least about 80%, at least about 90%, or at least about 100%, or greater.

In another aspect of any of these methods, the patient may be refractory to one or more existing cancer treatments. By “refractory” in this context is meant that the proliferative disease does not respond to treatment. The proliferative disease may be resistant at the beginning of treatment or it may become resistant during treatment.

The STAT3 inhibitor, Jak2 inhibitor, and interferon can be administered by any suitable method, as is known in the art. Pharmaceutical compositions suitable for the delivery of these agents and methods for their preparation will be readily apparent to those skilled in the art. Such compositions and methods for their preparation may be found, e.g., in Remington's Pharmaceutical Sciences, 19th Edition (Mack Publishing Company, 1995).

Pharmaceutical compositions for use in the present methods may be formulated according to techniques and procedures well-known in the art and widely discussed in the literature and may comprise any of the known carriers, diluents, or excipients. In one aspect, the compositions may be in the form of (sterile) aqueous solutions and/or suspensions of the pharmaceutically active ingredients, aerosols, ointments, and the like. Formulations which are aqueous solutions are most preferred. Such formulations typically contain for example, the STAT3 inhibitor itself, or interferon, water, and one or more buffers which act as stabilizers (e.g., phosphate-containing buffers) and optionally one or more preservatives.

Pharmaceutical compositions may include pharmaceutically acceptable salts of the STAT3 inhibitor, Jak2 inhibitor or interferon. For a review on suitable salts, see Handbook of Pharmaceutical Salts Properties, Selection, and Use by Stahl and Wermuth (Wiley-VCH, 2002). Suitable base salts are formed from bases which form non-toxic salts. Representative examples include the aluminium, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts.

Compositions to be used in the invention suitable for parenteral administration may comprise sterile aqueous solutions and/or suspensions of the pharmaceutically active ingredients preferably made isotonic with the blood of the recipient, generally using sodium chloride, glycerin, glucose, mannitol, sorbitol, and the like.

Compositions of the invention suitable for oral administration may, e.g., comprise the drug in sterile purified stock powder form preferably covered by an envelope or envelopes (enterocapsules) protecting from degradation in the stomach and thereby enabling absorption of these substances from the gingiva or in the small intestines. The total amount of active ingredient in the composition may vary from 99.99 to 0.01 percent of weight.

Pharmaceutical compositions of the STAT3 inhibitor, Jak2 inhibitor or interferon may be administered directly into the blood stream, into muscle, or into an internal organ. Suitable means for parenteral administration include intravenous, intra-arterial, intraperitoneal, intrathecal, intraparenchymal, intraventricular, intraurethral, intrasternal, intracranial, intramuscular, intrasynovial, and subcutaneous. Suitable devices for parenteral administration include needle (including microneedle) injectors, needle-free injectors, and infusion techniques.

Parenteral formulations are typically aqueous solutions which may contain excipients such as salts, carbohydrates, and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, e.g., by lyophilization, may readily be accomplished using standard pharmaceutical techniques well-known to those skilled in the art.

Formulations for parenteral administration may be formulated to be immediate and/or sustained release. Sustained release compositions include delayed, modified, pulsed, controlled, targeted and programmed release.

STAT3 and JAK2 inhibitors for use in the present invention may also be administered topically, (intra)dermally, or transdermally to the skin or mucosa. Examples of such pharmaceutical dosage forms include inter alia solutions, suspensions, dispersions, tinctures, gels, topical sprays, topical foams, gels, water-in-oil emulsions such as ointments, and oil-in water emulsions such as creams, lotions, and balms.

Typical carriers include alcohol, water, mineral oil, liquid petrolatum, white petrolatum, glycerin, polyethylene glycol, and propylene glycol. Penetration enhancers may be incorporated—see, e.g., Finnin and Morgan: J. Pharm. Sci. 88(10): 955-958, (1999). Other means of topical administration include delivery by electroporation, iontophoresis, phonophoresis, sonophoresis, and microneedle or needle-free injection (e.g., products sold under the trademarks, POWDERJECT™, BIOJECT™). Formulations for topical administration may be formulated to be immediate and/or modified release. Modified release formulations include delayed, sustained, pulsed, controlled, targeted and programmed release.

The term “gel”, as used herein, refers to a colloidal system in which a porous network of interconnected nanoparticles spans the volume of a liquid medium. In general, gels are apparently solid, jelly-like materials. Both by weight and volume, gels are mostly liquid in composition and thus exhibit densities similar to liquids, however have the structural coherence of a solid.

In one aspect of the invention, the pharmaceutical dosage form of the STAT3 or Jak2 inhibitor is a hydrogel. A “hydrogel”, as used herein, refers to a gel made of one or more cross-linked water-swellable (hydrophilic) gel-forming polymers such as polysaccharides or polyacrylic acid derivatives. The gel-forming polymers may be naturally occurring polymers, synthetic polymers or mixtures thereof. Hydrogels may comprise more than 99% water. When applied to the skin the water bound in such a hydrogel does not evaporate as fast as from a solution. Due to the thus prolonged contact period the skin becomes moistened which, in turn, results in an improved susceptibility for the uptake of active ingredients present in the hydrogel (i.e. an increased penetration through the skin). This phenomenon is also referred to as “occlusion effect”.

Typically, such gel-forming polymers have an average molecular weight of 1000 to 50000 Dalton, preferably of 1000 to 30000 Dalton. A hydrogel of the invention may also be characterized by its rheological properties. Typically, it has an initial shear modulus of 0.005 to 200 kPa, preferably of 0.05 to 100 kPa. The “shear modulus”, also referred to as the modulus of rigidity, is defined as the ratio of shear stress to the shear strain and provides a measure for the strength of a given material. Additionally or alternatively, it may also be possible to characterize a hydrogel by its flow behavior such as by its viscosity coefficient η as determined by the flow models of Bingham, Casson, Herschel-Bulkley and Ostwald, respectively, all of them well known in the art (see, e.g. Gosh, T. K. et al. (1997) Transdermal and topical drug delivery systems. CRC Press, Boca Raton, Fla., USA; Fairclough, J. P. A., and Norman A. I. (2003) Annu. Rep. Prog. Chem., Sect. C: Phys. Chem. 99, 243-276).

In another aspect, the hydrogel comprises one or more gel-forming polymers in a total amount of 0.1% to 15% (w/w) based on the total weight of the hydrogel. In one aspect, the one or more gel-forming polymers are selected from the group consisting of cellulose derivatives, polyacrylic acid derivatives, and gums. Examples of cellulose derivatives include inter alia methylcellulose, ethylcellulose, hydroxyethyl cellulose, and carboxymethyl cellulose. Examples of polyacrylic acid derivatives include inter alia polyacrylic acid, polymethylacrylate, and polyethylacrylate. Examples of gums (also referred to as “rubbers”) include inter alia agar, alginic acid, glucomannan, arabic gum, sodium alginate, and tragacanth.

In some embodiments, the inventive hydrogel does not comprise any lipids, that is it is a “fat-free” hydrogel. Typically, the hydrogels of the invention comprise at least 75% (w/w) water, and preferably they comprise at least 80% (w/w) water.

In other aspects of the invention, the pharmaceutical dosage form of the STAT3 or JAK2 inhibitor is an oil-in-water emulsion. The term “oil-in-water emulsion”, as used herein, refers to formulations which are composed of small droplets of a lipid phase (e.g., an oil) dispersed in a continuous aqueous phase. An “emulsion” is a mixture of two immiscible (i.e. not mixable) substances. One substance (the dispersed phase) is dispersed (i.e. distributed) in the other (the continuous phase) by the presence of one or more emulsifying agents. In general, oil-in-water emulsions are more comfortable and pharmaceutically/cosmetically acceptable as compared to water-in-oil emulsions (such as an ointment) as they are less greasy when applied on the skin and more easily washed off when using water. By employing such an oil-in-water emulsion the penetration of amphiphilic compounds such as the STAT3 inhibitors of the invention through the skin is improved as compared to formulations having only an aqueous phase, since the presence of a lipid phase is assumed to aid in crossing the hydrophobic core of biological membranes.

In some aspects, oil-in-water-emulsions of the invention are selected from the group consisting of creams, lotions, and balms. These formulations primarily differ with regard to their respective viscosities. A cream is a semi-solid emulsion, that is it has a medium viscosity. In contrast, a lotion is a low- to medium-viscosity preparation intended for application to unbroken skin. Finally, a balm (also referred to as liniment) has a similar viscosity as a lotion (i.e. being significantly less viscous than a cream) but unlike a lotion a balm is applied with friction, that is a liniment is always rubbed in.

In one aspect, the oil-in-water emulsions according to the invention comprises one or more emulsifiers in a total amount of 0.5% to 15% (w/w) based on the total weight of the dosage form. Whether an emulsion turns into a water-in-oil emulsion or an oil-in-water emulsion depends on the volume fraction of both phases and on the type of emulsifier. Generally, the Bancroft rule applies: emulsifiers and emulsifying particles tend to promote dispersion of the phase in which they do not dissolve very well. In other words, the phase in which an emulsifier is more soluble constitutes the continuous phase. Thus, for the preparation of oil-in-water emulsions water-soluble emulsifiers are typically used.

In one aspect, the one or more emulsifiers are selected from the group consisting of sorbitan esters (also referred to as Span®), polyoxyethylene sorbitan esters (also referred to as polysorbates; Tween®), and glyceryl esters. Examples of sorbitan esters include inter alia sorbitan monooleate, sorbitan monostearate, sorbitan monolaurate, sobitan trioleate, and sorbitan tristearate. Examples of polyoxyethylene sorbitan esters include polyethylene glycol (PEG) sorbitan esters such as inter alia PEG-(5)-sorbitan monooleate, PEG-(4)-sorbitan monostearate, PEG-(4)-sorbitan monolaurate, PEG-sobitan trioleate, and PEG-sorbitan tristearate. Examples of glyceryl esters include inter alia glyceryl monostearate, glyceryl monolaurate, and glyceryl tristearate.

Other emulsifiers that can be used in the present invention include inter alia lecithin, cholesterol, phosphatidylglycerols, alkyl alcohols, poloxamers (also referred to as Pluronic®/Synperonic®), poloxamin (also referred to as Tetronic®), sodium laurylsulfate, sodium cetylstearylsulfate, and potassium oleate.

The pharmaceutical dosage forms of the present invention comprise at least one pharmaceutically acceptable excipient. The term “pharmaceutically acceptable excipient”, as used herein denotes any substance used for the preparation of pharmaceutical dosage forms such as carrier materials, wetting agents, preservatives, buffers, solvents or solubilizers, agents for achieving a depot effect, and other adjuvants, all of them well known in the art (cf. the references cited below).

All these topical pharmaceutical dosage forms as well as methods for their preparation are well established in the art (see, for example, Niedner, R., and Ziegenmeyer, J. (1997) Dermatika. Therapeutischer Einsatz, Pharmakologie and Pharmazie. Wissenschaftliche Verlagsgesellschaft mbH, Stuttgart, Germany; Gennaro, A. L. and Gennaro, A. R. (2000) Remington: The Science and Practice of Pharmacy, 20th Ed., Lippincott Williams & Wilkins, Philadelphia, Pa.; Niazi, S. K. (2004) Handbook of Pharmaceutical Manufacturing Formulations, CRC Press, Boca Raton, Fla.).

In one aspect, the topical formulations of the present invention comprise a pharmaceutically acceptable carrier or diluent and an effective amount of a STAT3, or JAK2 inhibitor. These topical formulations can be in any suitable form known to the person skilled in this field and can, for example, take the form of an ethanol solution, cleansing foam, cleansing cream, skin gel, skin lotion, shampoo gel, cream shampoo or the like.

Topical formulations are prepared by adding an exemplified compound to a base well known to those skilled in the art; for example, suspending agents (examples include gum arabic, tragacanth, methyl cellulose, sodium carboxymethylcellulose, hydroxyethyl cellulose, hydroxypropyl cellulose, sodium alginate and bentonite), emulsifying agents (examples include triethanolamine, sodium lauryl sulfate, sorbitan sesquioleate, polysorbate 80 and stearic acid polyoxyl 40), moistening agents (examples include sorbitol, ethylene glycol, propylene glycol, butylene glycol and glycerin), preservatives (examples include methyl paraoxybenzoate, ethyl paraoxybenzoate, propyl paraoxybenzoate and butyl paraoxybenzoate) or solvents (examples include water; alcohols such as ethanol, isopropyl alcohol, propylene glycol, cetanol and isostearyl alcohol; hydrocarbons such as natural fats and oils, waxes and liquid paraffin; aliphatic acids such as stearic acid, isostearic acid, oleic acid and linoleic acid; and esters such as isopropyl myristate) or a mixture thereof.

The amount of the STAT3 inhibitor or derivative thereof or JAK2 inhibitor or derivative thereof locally administered will vary depending on the condition, age or the like of the patient. It is desirably administered at a concentration of about 0.01 mg/ml formulation, about 0.1 mg/mI formulation, about 1 mg/mI formulation, about 10 mg/mI formulation, about 20 mg/mI formulation, about 30 mg/mI formulation, or about 50 mg/mI formulation and administered in a single dose or in several divided doses a day.

Interferon is typically administered by intramuscular or subcutaneous injection, and can be administered in a dose of between 3 and 10 million units, with 3 million units being preferred in one embodiment. Representative doses include for Hepatitis B; INTRON A: 30-35 million international units (MIU) per week, administered subcutaneously or intramuscularly, for up to 16 weeks.

Hepatitis C; INTRON A or ROFERON: 3 MIU, administered subcutaneously or intramuscularly, three times per week for up to 24 months; PEG-Intron monotherapy: 1 μg/kg/week, administered subcutaneously, for one year; PEGASYS monotherapy: 180 μg/week, administered subcutaneously, for up to 48 weeks.

High risk melanoma (adjuvant to surgery); INTRON A: 20 m2 MIU, administered subcutaneously, 5 times per week for 4 weeks (induction), and 10 m2 MIU 3 times per week for 48 weeks (maintenance). PEG-Intron (experimental regimen): 6 μg/kg/week, administered subcutaneously, once per week for 8 weeks (induction course), and 3 μg/kg/week for 252 weeks (maintenance course).

Hairy cell leukemia; INTRON A: 2 MIU/m2, administered subcutaneously or intramuscularly, daily for up to 6 months.

Chronic myelogenous leukemia; INTRON A: 4-5 MIU/m2, administered subcutaneously, daily to hematological remission. PEG-Intron: 6 μg/kg/week administered subcutaneously for up to a year.

AIDS-related Kaposi's sarcoma; INTRON A: 30 MHJ/m2, administered subcutaneously or intramuscularly, 3 times per week for up to 16 weeks.

Renal cell carcinoma; INTRON A: 9-10 3VHU, administered subcutaneously or intramuscularly, 3 times per week for 4 weeks. PEG-Intron (experimental regimen): 4.5-6 μg/kg/week.

Doses of interferon are administered on a regular schedule, which can vary from 1, 2, 3, 4, 5, or 6 times a week, to weekly, biweekly, every three weeks, or monthly. A typical dose of interferon that is currently available is provided weekly, and that is a preferred dosing schedule for interferon, according to the present invention. The dose amount and timing can be varied according to the preferences and recommendations of the physician, as well as according to the recommendations for the particular interferon being used, and it is within the abilities of those of skill in the art to determine the proper dose.

In one aspect of these methods the STAT3 inhibitor and interferon are administered to a patient with a proliferative disease. The term “proliferative disease” refers to a cancer, (and/or any metastases) or hyperproliferative condition, such as a leukemia, lymphoma or multiple myeloma. The term also includes benign tumors, malignant tumors, rheumatoid arthritis, psoriasis, ocular angiogenesis diseases, Osler-Webber Syndrome, myocardial angiogenesis, plaque neovascularization, graft and post-angioplasty stenosis, telaniectasia, hemophiliac joints, angiofibroma, wound granulation, intestinal adhesions, atherosclerosis, scleroderma, hypertrophic scars, cat scratch disease, and Heliobacter pylori ulcers./or metastasis.

Such diseases include for example, breast cancer; lung cancer, including non-small cell lung cancer (NSCLC) and small-cell lung cancer (SCLC); gastrointestinal cancer, including esophageal, gastric, small bowel, large bowel, rectal and colon cancer; CNS cancer including brain cancer and cancer metastatic to CNS; sarcoma, such as those involving bone, cartilage, soft tissue, muscle, blood and lymph vessels; ovarian cancer; myeloma; female cervical cancer; endometrial cancer; head and neck cancer; mesothelioma; renal cancer; uteran; bladder and urethral cancers; hematological malignancies including leukemia, lymphoma and myeloma; prostate cancer; skin cancers, including squamous cell carcinomas (SCC), basal cell cancers, cutaneous T-cell lymphomas, primary cutaneous B cell lymphomas, Dermatofibrosarcoma protuberans, Merkel cell carcinoma, Kaposi's sarcoma, keratoacanthoma, and melanoma. Where a tumor, a tumor disease, a carcinoma or a cancer are mentioned, also metastasis in the original organ or tissue and/or in any other location are implied alternatively or in addition, whatever the location of the tumor.

In one aspect of any of these methods, the melanoma is CNS melanoma. In another aspect of any of these methods, the patient has Leptomeningeal disease (LMD). In another aspect of any of these methods, the patient has stage III melanoma. In another aspect of any of these methods, the patient has stage IV melanoma.

In another aspect of any of these methods, the proliferative disease may be refractory to one or more existing cancer treatments. By “refractory” in this context is meant that the proliferative disease does not respond to treatment. The proliferative disease may be resistant at the beginning of treatment or it may become resistant during treatment.

### II. STAT3 Inhibitors

Inhibitors of STAT3 (also referred to herein as inhibitors of p-STAT3, phosphorylated or activated STAT3) are useful in treating a wide variety of cancers because these inhibitors provide tumor cytotoxic effects—whether acting directly or indirectly on the activation of STAT3 and/or whether the inhibitor prevents activation of STAT3, upstream or downstream in its pathway.

However, while these inhibitors can have a significant impact on patients with primary and metastatic tumors, STAT3 blockade agents (also referred to sometimes as a “STAT3 inhibitors”) have multiple mechanisms of activity and potentially conflicting effects. In short, the various targets of STAT3 blockade agents (even if selective) include molecules in the STAT3 activation pathway of both tumor cells and immune cells. As such, this effect can adversely impact the potential wide spread uses of STAT3 inhibitors.

The activation of the p-STAT3 pathway is useful to prevent anti-tumor immune activity as inflammatory conditions can initate or promote oncogenic transformation. Yu, H. et al, STATs in Cancer Inflammation and Immunity: a Leading Role for STAT3, Nature, Vol. 9, 798-809 (November 2009) (where signalling between STAT3 and NF-kB signalling is noted as interconnected). As discussed by Yu, overexpression or persistent activation of growth factor receptors together with oncogenic mutations in the receptor associated JAK family members may cause some types of cancer. Yu, H. et al, STATs in Cancer Inflammation and Immunity: a Leading Role for STAT3, supra, discussed in detail at pages 799 to 807, incorporated herein by reference. The tyrosine kinase receptors and non-receptor tyrosine kinases such as SRC can be activated by extrinsic pathways such as factors associated with inflammation such as UV radiation or sunlight, chemical carcinogens, infection, stress and cigarette smoke. In turn, the tyrosine kinases induced by both extrinsic and instrinsic pathways phosphorylate STAT3 which in turn forms dimers that translocate to the nucleus where gene expression is directly regulated. In addition to upregulating genes, STAT3 will induce the expression of many cytokines, chemokines and other mediators such as IL-6 and cyclooxygenase 2 that are associated with cancer-promoting inflammation. Most importantly, the receptors for many of the cytokines further active STAT3.

Inhibitors of STAT3 useful in connection with the methods provided herein include direct and indirect inhibitors of STAT3. Representative inhibitors of STAT3 are disclosed in Examples 1 to 63 of the present application. These compounds can be prepared by following the procedures described in WO 2005058829 (pages 22-29), US 20050277680, U.S. Pat. No. 7,745,468 (column 20, line 1 to column 25, line 12), WO 2007115269 (pages 40-52), US 20070232668 (pages 17-22), and WO 2010005807 (paragraphs [0191]-[0201]),

Potential indirect inhibitors of STAT3 phosphorylation include inhibitors of upstream activators like growth factors, cytokines, src, Tyk2 and Janus kinases (this includes Jak2, Jak3, and Tyk2 inhibitors). Key activators of STAT3 include IL-6, IL-10, IL-23, IL-11 and OSM. Molecules upregulated by STAT3 which may be modulated by a STAT3 inhibitor include but are not limited to BCL-X_{L}, MYC, BIRC5, MMP9, MMP2, HIFα, ICAM1, TWIST1, VIM, MCL1, HSP70 and HSP90, IL-10, VEGF, FGF2 (also known as BFGF), COX2 CXCL12 (also known as SDF1), IL-11, IL-23, IL-17, and IL6. Molecules downregulated by STAT3 include IL-6, IL-12A (also known as P35), CD80, CD86, CXCL10 (also known as IP-10), IFNα, IFNβ, CCL5, NOS2, IL-8, IL-113, and CCL2 (also known as MCP1).

More specifically potential indirect inhibitors of STAT3 include the Jak2 inhibitors currently under clinical trials such as: INCB018424 by Incyte; TG101348 by TargeGen; CEP-701 (lestaurtinib) by Cephalon; AZD1480 by AstraZeneca; XL019 by Exelixis; CYT-387 by Cytopia; SGI-1252 by SuperGen; and SB1518 by S*BIO. The Jak2 Inhibitors in preclinical development can also be useful as an indirect inhibitor of STAT3 and include: AG490; Tkip; Z3; TG101209; and C7. Furthermore, non-specific inhibitors of Jak2 can be useful and currently including: Go6976; Erlotinib; Atiprimod; CP-690,550; AT9283; and MK-0457.

Discovery of Small Molecule Inhibitors of p-STAT3

A variety of small molecule inhibitors of STAT3 have been devised. Several approaches have been undertaken to interfere with the signaling of STAT3, including blocking the ligand-receptor interaction and activation sites of STAT3, dimerization, nuclear translocation, DNA binding, and gene transcription. The STAT3 inhibitor AG490 has been studied extensively; however, its low potency (IC₅₀>50 μM in vitro) and lack of biostability prevent its use in vivo. Therefore, we have designed and developed a series of small molecular STAT3 inhibitors based on the caffeic acid benzyl ester/AG490 scaffold that blocks STAT3 phosphorylation. The WP (Waldemer Priebe) compounds, such as WP1066 shown in FIG. 3), WP1220, and WP1193, were devised utilizing a combination of molecular modeling and medicinal chemistry to synthesize inhibitors that optimally inhibit the Jak2/STAT3 interaction and subsequent phosphorylation of STAT3 at tyrosine⁷⁰⁵ and STAT-5 at tyrosine⁶⁹⁴. Priebe, W, Donato, N, Talpaz, M, Fokt, I, Szymanski, S, WO04104013214 (2004); Priebe W, Fokt I, Szymanski S, et al., Design, Synthesis And Structure-Activity Relationships Of Novel Jak2/STAT3 Signaling Inhibitors, Proc 97th Amer Assoc Cancer Res Annual Meeting. Washington, D.C. (2006); Madden T, Kazerooni R, Myer J, et al., The Preclinical Pharmacology Of WP1066, A Potent Small Molecule Inhibitor Of The JAK2/STAT3 Pathway, Proc 97th Amer Assoc Cancer Res Annual Meeting. Washington, D.C. (2006); Priebe, W, Donato, N, Talpaz, M, Szymanski, S, Fokt, I, Levitki, A, WO04US0041712 (2005), US2003000528877P (2003); Priebe, W, Fokt, I, Szymanski, S, Madden, T, Myers, J, Conrad, C, WO07115269A2 (2007), US20070232668A1 (2007).

JSI-124 (cucurbitacin I) was identified from the National Cancer Institute Diversity Set using a high-throughput STAT3 cytoblot assay that was selective for the Jak/STAT3 pathway. Sebti, S M, Jove, R, US2004000472056 (2004), WO02US0011157 (2004). JSI-124 is a member of the cucurbitacin family of compounds that were isolated from the plant families Cucubitaceae and Cruciferae. The mechanism by which JSI-124 inhibits p-STAT3 has not been definitively defined but could be secondary to the down-regulation of p-STAT3 through the promotion of the protein phosphatase activities of SHP-1 and SHP-2 or activation of physiological inhibitors. Stofega M R, Wang H, Ullrich A, Carter-Su C, Growth Hormone Regulation Of SIRP And SHP-2 Tyrosyl Phosphorylation And Association, J Biol Chem 1998; 273: 7112-7117; Schaper F, Gendo C, Eck M, et al., Activation Of The Protein Tyrosine Phosphatase SHP2 Via The Interleukin-6 Signal Transducing Receptor Protein gp130 Requires Tyrosine Kinase Jak1 And Limits Acute-Phase Protein Expression, Biochemical Journal 1998; 335:557-565; Turkson J, Jove R, STAT Proteins: Novel Molecular Targets For Cancer Drug Discovery, Oncogene 2000; 19:6613-6626.

The most recently defined and patented small molecular inhibitors of p-STAT3 were identified using structure-based virtual screening with computer modeling of the binding configuration during STAT3 dimerization. Siddiquee K, Zhang S, Guida W C, et al., Selective Chemical Probe Inhibitor Of STAT3, Identified Through Structure-Based Virtual Screening, Induces Antitumor Activity, Proc Natl Acad Sci USA 2007; 104:7391-7396. Cucurbitacin Q (NSC 135075) was obtained from the National Cancer Institute and, as initially characterized, is actually with acnistin. Sebti, S M, WO07US0002827 (2007); Sun J, Blaskovich M A, Jove R, Livingston S K, Coppola D, Sebti S M, Cucurbitacin Q: A Selective STAT3 Activation Inhibitor With Potent Antitumor Activity, Oncogene 2005; 24:3236-3245. S31-201 (NSC 74859) was similarly identified from the National Cancer Institute chemical library using structure-based virtual screening. Finally, a peptidomimetic inhibitor, ISS 610, was used in a structure-based model to design and characterize an oxazole-based peptidomimetic, S31-M2001. Siddiquee K A, Gunning P T, Glenn M, et al., An Oxazole-Based Small-Molecule Stat3 Inhibitor Modulates STAT3 Stability And Processing And Induces Antitumor Cell Effects, ACS Chemical Biology [Electronic Resource] 2007; 787-798. The decoy anti-sense STAT3 oligonucleotides can interfere at the level of DNA binding or to RNA transcripts but are not a focus of this review.

Properties of Small Molecule Inhibitors of p-STAT3

Of central importance to the treatment of CNS gliomas and metastasis is the use of agents with sufficient CNS permeability and subsequent cytotoxic activity, of which there is direct evidence for WP1066 and indirect evidence for JSI-124. To determine the physiological doses of WP1066 that can be achieved in vivo, CD₁ mice were given WP1066 as an intravenous bolus (10 or 40 mg/kg) or by oral gavage (40 mg/kg) and killed at various time points up to 24 hours after treatment. The mean peak plasma concentrations achieved using the 10- and 40-mg/kg doses were 1.05 μM and 4.31 μM, respectively. Oral bioavailability studies yielded mean peak plasma concentrations greater than 2 μM. When WP1066 was delivered intraperitoneally at a dose of 100 mg/kg or intravenously at a dose of 10 mg/kg every other day for up to 2 weeks, plasma concentrations exceeded 1 μM, and CNS concentrations (with an intact blood-brain barrier) exceeded 62 μg/g of tissue (0.185 μM); concentrations in U87-MG malignant glioma-bearing animals exceeded 362 μg/g (1.08 μM). Although formal bioavailability studies of JSI-124 have not been published, it too appears to have sufficient CNS penetration properties, as evidenced by prolonged survival in murine models with intracerebral tumors treated with JSI-124. Fujita M, Zhu X, Sasaki K, et al., Inhibition Of STAT3 Promotes The Efficacy Of Adoptive Transfer Therapy Using Type-1 CTLs By Modulation Of The Immunological Microenvironment In A Murine Intracranial Glioma, J Immunol 2008; 180:2089-2098.

The toxicological properties of the small molecule inhibitors of p-STAT3 appear favorable. In the case of WP1066, we have noted localized inflammatory responses with intraperitoneal or intravenous administration, which can be easily overcome by administration via oral gavage. Histological examination of the systemic organs of mice treated with either WP1006 or JSI-124 have failed to reveal any major toxicities. Because p-STAT3 blockade agents are potent immune activators, nonspecific immune reactivity in the CNS remains a consideration. Therefore, we performed Luxol fast blue staining of the CNS axis in C57BL/6J mice that were treated with WP1066 and found no evidence of induction of autoimmunity. No focal plaques of demyelination or significant infiltration with macrophages was observed in areas without tumor or in any animal given WP1066. Furthermore, there was no evidence of induced autoimmunity in animals treated with JSI-124. Fujita M, Zhu X, Sasaki K, et al., Inhibition Of STAT3 Promotes The Efficacy Of Adoptive Transfer Therapy Using Type-1 CTLs By Modulation Of The Immunological Microenvironment In A Murine Intracranial Glioma, J Immunol 2008; 180:2089-2098. This lack of induced autoimmunity may be due to STAT3's role as the key regulator of the generation of Th17 cells, which are primary immune cell mediators of autoimmunity. Thus, the inhibitors of p-STAT3 may also be inhibiting the mediators of CNS autoimmunity-Th 17 responses.

Antitumor Properties of Small Molecule Inhibitors of P-STAT3

The small molecule inhibitors of p-STAT3 have demonstrated activity against a wide variety of cancers. For example, cucurbitacin Q/with acnistin suppressed in vivo growth of human lung cancer xenografts. Sun J, Blaskovich M A, Jove R, Livingston S K, Coppola D, Sebti S M. Cucurbitacin Q, A Selective STAT3 Activation Inhibitor With Potent Antitumor Activity, Oncogene 2005; 24:3236-3245. S31-301 has been shown to inhibit growth and induce apoptosis preferentially in breast carcinoma cell lines with constitutively active STAT3 and to suppress the in vivo growth of human breast tumor xenografts. Siddiquee K, Zhang S, Guida W C, et al., Selective Chemical Probe Inhibitor Of STAT3, Identified Through Structure-Based Virtual Screening, Induces Antitumor Activity, Proc Natl Acad Sci USA 2007; 104:7391-7396. Similarly, S31-M2001 has been shown to suppress subcutaneous growth of human breast cancer xenografts. Siddiquee K A, Gunning P T, Glenn M, et al., An Oxazole-Based Small-Molecule Stat3 Inhibitor Modulates STAT3 Stability And Processing And Induces Antitumor Cell Effects, ACS Chemical Biology [Electronic Resource] 2007; 787-798. JSI-124 has been shown to have in vitro activity against lymphoma and cervical cancer. Shi X, Franko B, Frantz C, Amin H M, Lai R, JSI-124 (Cucurbitacin I)Inhibits Janus Kinase-3/Signal Transducer And Activator Of Transcription-3 Signalling, Downregulates Nucleophosmin-Anaplastic Lymphoma Kinase (ALK), And Induces Apoptosis In ALK-Positive Anaplastic Large Cell Lymphoma Cells, Br J Haematol 2006; 135:26-32; Chen C L, Hsieh F C, Lieblein J C, et al., Stat3 Activation In Human Endometrial And Cervical Cancers, Bri J Cancer 2007; 96:591-599. Furthermore, JSI-124 can inhibit the in vivo growth of human breast carcinoma and syngeneic murine melanoma, but not in carcinomas that lack constitutive expression of p-STAT3. Blaskovich M A, Sun J, Cantor A, Turkson J, Jove R, Sebti S M, Discovery Of JSI-124 (Cucurbitacin I), A Selective Janus Kinase/Signal Transducer And Activator Of Transcription 3 Signaling Pathway Inhibitor With Potent Antitumor Activity Against Human And Murine Cancer Cells In Mice, Cancer Res 2003; 63:1270-1279.

WP1066 inhibits both constitutive and induced STAT3. WP1066 administered intraperitoneally (40 mg/kg every other day in dimethyl sulfoxide: polyethylene glycol) for 28 days has demonstrated statistically significant suppression of tumor growth in nude mice with flank-bearing head and neck carcinoma (MDA1986), pancreatic cancer (MiaPaca2), bladder cancer, glioma (U-87), B-cell non-Hodgkin's lymphoma and myeloma, chronic myelogenous leukemia and acute myelogenous leukemia. Treatment of established tumors in vivo with WP1066 resulted in decreased tumor proliferation, volume, and angiogenesis/vascular proliferation, as detected using CD31 staining. Ferrajoli A, Faderl S, Van Q, et al., WP1066 Disrupts Janus Kinase-2 And Induces Caspase-Dependent Apoptosis In Acute Myelogenous Leukemia Cells, Cancer Res 2007; 67: 11291-1129; Kupferman M E, Zhou G, Zhao M, et al., A Novel Inhibitor Of STAT3 Signaling In Head And Neck Squamous Cell Carcinoma, Proc 97th Amer Assoc Cancer Res Annual Meeting. Washington, D.C. (2006); Bao J J, Fokt I, Szymanski S, Priebe W, Inhibition Of Constitutively Active STAT3 By WP1066 Suppresses Proliferation And Induces Apoptosis In Pancreatic Cancer Cells, Clin Cancer Res 2005; 11: 9026S-27S; Guha S, Chakraborty A, Szymanski S, et al., WP1066, A Potent Inhibitor Of Jak2/STAT3 Pathway Inhibits Pancreatic Tumor Growth Both In Vitro And In Vivo, Proc 98th Amer Assoc Cancer Res Annual Meeting. Los Angeles, Calif. (2007); Chakraborty A, Guha S, Helgason T, et al., A Novel Jak2/STAT3 Pathway Inhibitor Promotes Apoptosis And Blocks Growth Of Bladder Cancer Cells, Proc 98th Amer Assoc Cancer Res Annual Meeting. Los Angeles, Calif. (2007); Iwamaru A, Szymanski S, Iwado E, et al., A Novel Inhibitor Of The STAT3 Pathway Induces Apoptosis In Malignant Glioma Cells Both In Vitro And In Vivo, Oncogene 2006; 26: 2435-2444; Kong L-Y, Kapuria V, Bartholomeusz G, Priebe W, Talpaz M, Donato N, Antitumor Activity And Mechanism of Action Of A Novel STAT3 Inhibitor, WP1066, Against Human B-Cell Non-Hodgkin's Lymphoma And Multiple Myeloma, Blood 2005; 106:429 A, 1489 Part 1; Samanta A, Kantarjian H, Priebe W, Arlinghaus R, Cross Talk Between Jak2 And Lyn In Bcr-Abl Signaling Pathway In Cells From Imatinib-Sensitive And Resistant Chronic Myelogenous Leukemia (CML), Proc 98th Amer Assoc Cancer Res Annual Meeting. Los Angeles, Calif. (2007). Western blotting from WP1066-treated in vivo tumors has shown inhibition of p-STAT3 and downstream molecules such as VEGF, survivin, and BCL-XL.

In murine models of subcutaneous malignant glioma xenografts (U-87 and U-373) during a 30-day follow-up, there was significant inhibition of tumor growth in the animal group treated with systemic intraperitoneal administration of WP1066. Immunohistochemical analysis of the excised tumors demonstrated that p-STAT3 was inhibited for at least 3 weeks after the final WP1066 injection. Iwamaru A, Szymanski S, Iwado E, et al., A Novel Inhibitor Of The STAT3 Pathway Induces Apoptosis In Malignant Glioma Cells Both In Vitro And In Vivo. Oncogene 2006; 26: 2435-2444. We have also found marked growth suppression of human melanoma xenografts and syngeneic murine melanoma with WP1066 administered via oral gavage. To determine whether treatment with WP1066 is efficacious against established syngeneic intracerebral tumors, C57BL/6J mice with melanoma tumors (B16) were treated by oral gavage with WP1066 starting on day 3 after tumor implantation. Median survival durations and rates were enhanced markedly when the mice with established tumors were treated with WP1066, and 80% of WP1066-treated animals survived long-term compared with 0% of the vehicle control-treated animals, indicating that small molecule inhibitors of p-STAT3 can exert antitumor activity within the CNS. Kong L Y, Abou-Ghazal M K, Wei J, et al., A Novel Inhibitor Of STAT3 Activation Is Efficacious Against Established Central Nervous System Melanoma And Inhibits Regulatory T Cells, Clin Cancer Res (Submitted).

Immune Modulatory Properties of Small Molecule Inhibitors of p-STAT3. On the basis of the crucial role that p-STAT3 plays in mediating immune suppression, the development of small molecule inhibitors as immune therapeutics is logical. Nefedova et al., demonstrated that JSI-124 could overcome the dendritic cell differentiation block induced by tumors. The treatment of immature dendritic cells with JSI-124 resulted in the up-regulation of MHC class II and co-stimulatory molecules, resulting in functional T cell stimulation/activation secondary to a combination of dendritic cell maturation and the induction of apoptosis in immature dendritic cells. Nefedova Y, Cheng P, Gilkes D, et al., Activation Of Dendritic Cells Via Inhibition Of Jak2/STAT3 Signaling, J Immunol 2005; 175: 4338-4346. Furthermore, using an in vivo sarcoma model in which JSI-124 had minimal direct tumor cytotoxicity, the authors demonstrated potent enhancement of dendritic cell vaccination in combination with JSI-124 resulting in marked tumor suppression that persisted for more than 4 weeks. Nefedova Y, Nagaraj S, Rosenbauer A, Muro-Cacho C, Sebti S M, Gabrilovich D I, Regulation Of Dendritic Cell Differentiation And Antitumor Immune Response In Cancer By Pharmacologic-Selective Inhibition Of The Janus-Activated Kinase 2/Signal Transducers And Activators Of Transcription 3 Pathway, Cancer Res 2005; 65:9525-9535.

Fujita et al. demonstrated that the administration of JSI-124 in vivo promoted a Th1 (cytotoxic effector) phenotype and enhanced glioma-infiltrating immune cells. Fujita M, Zhu X, Sasaki K, et al., Inhibition Of STAT3 Promotes The Efficacy Of Adoptive Transfer Therapy Using Type-1 CTLs By Modulation Of The Immunological Microenvironment In A Murine Intracranial Glioma, J Immunol 2008; 180:2089-2098. Immune-competent mice with intracerebral tumors treated with JSI-124 had prolonged survival, but this efficacy was not observed in an immune-incompetent background, indicating that the immune system played a role in the in vivo effect f JSI-124. Furthermore, when JSI-124 was combined with adoptive transfer of type I cytotoxic T lymphocytes, survival was further enhanced compared with treatment with either modality alone. The authors concluded that the inhibition of STAT3 could reverse the immune-suppressive immune microenvironment and promote the efficacy of adoptive transfer therapy. Id. We have also found that the marked in vivo efficacy of WP1066 in established intracerebral tumors is ablated in an immune-incompetent background, indicating that immune responses play a pivotal role in the cytolytic clearance of CNS tumors using these agents.

We recently have shown that blocking STAT3 activity by treatment with WP1066 in microglia obtained from the CNS of patients with cancer upregulates co-stimulatory molecules (e.g., CD80, CD86). The expression of co-stimulatory molecules is necessary for T-cell proliferation, and if a T cell encounters APCs, such as microglia/microphages, without co-stimulatory molecules present, the T cells will be rendered anergic. WP1066 also induces systemic APCs to produce pro-inflammatory cytokines (IL-2, IL-4, IL-12, and IL-15) essential for T-effector responses, even in immune-suppressed patients such as those with malignant gliomas, patients treated with steroids, and stage IV cancer patients. Finally, WP1066 inhibits immune-suppressive cytokines such as TGF-β and induces the activation and proliferation of T cells, indicating that STAT3 blockade is a potent approach to modulating both the systemic and local tumor immune microenvironment. Of note, this potent immune activation occurred in immune cells obtained from patients with disease refractory to other conventional immune activators, such as toll-like receptor agonists.

### III. Interferons

As used herein, the term “type I interferon” means any interferon protein (abbreviated “IFN”) that is capable of binding to and activating the type 1 human interferon receptor IFNAR (also referred to as the IFN α/β receptor complex), which comprises two transmembrane subunits, IFNAR1 and IFNAR2 (see Domanski, P., et al., The type-I interferon receptor. The long and short of it., Cytokine Growth Factor Rev. 7:143-151 (1996) Brierley, M. M. et al., IFN α/β; receptor interactions to biologic outcomes: understanding the circuitry. J. Interferon Cytokine Res. 22:835-845 (2002); and Stark, G. R. et al. How Cells respond to Interferons. Ann. Rev. Biochem. 67:227-264 (1998). Upon binding to a type I interferon, the IFNAR1 and IFNAR2 oligomerize and activate signal transduction via intracellular Janu-associated kinases, signal transducers and activators of transcription (JAK/STAT pathway) as well as other pathways in certain cell types (e.g. IRS1/2/PI3K, p38, CrkL, and vav).

Type I IFNs consist of nine distinct classes. In addition to IFN-α, IFN-β, there are other IFNs Type I that bind to the type I receptor, namely IFN-δ, IFN-ε, IFN-κ, IFN-ω, IFN-ν, IFN-τ and IFN-ζ. Type I interferons useful in practicing the present invention include, but are not limited to, all naturally-occurring subtypes of the type I interferons that are expressed in human cells: IFN-α, IFN-β, IFN-ω, and IFN-ω(see Chen, J. et. al., Diversity and Relatedness Among the Type I Interferons. J. of Interferon; Cytokine Res. 24:687-698 (2004). Preferably, the type I interferon is a human IFN-α. Particularly preferred human IFN-α subtypes are α-2a (GenBank Accession Number NP_{—}000596) and α-2b (GenBank Accession Number AAP20099), which may be recombinantly produced as mature polypeptides as described in U.S. Pat. No. 6,610,830. Mature IFN-α-2a is marketed as ROFERON® A by Hoffmann-LaRoche, Nutley, N.J. and mature IFN-α 2b is marketed as EMTRON® A by Schering Corporation, Kenilworth, N.J. Another recombinant IFN-α; that is suitable for use in the present invention is IFN-α 2c marketed as BEROFOR® by Boehringer Ingelheira GmbH, Germany. IFN-α subtypes which may be used in the present invention also include IFN-α la, marketed as AVONEX® by Biogen Idee and IFN-α-lb, marketed as BETAFERON® in Europe by Schering AG. Other exemplary interferons, suitable for use in the current invention include oral interferon alpha (Amarillo Biosciences), BLX-883 (Locteron; Biolex Therapeutics/OctoPlus), MULTIFERON® (Viragen), and omega interferon (Intarcia Therapeutics).

The term “type I interferon” also includes biologically active polypeptide fragments of type I interferons, as well as chimeric or mutant forms of type I interferons in which sequence modifications have been introduced, for example to enhance stability, without affecting their ability to activate the IFNAR, such as consensus interferons as described in U.S. Pat. Nos. 5,541,293, 4,897,471 and 4,695,629, and hybrid interferons containing combinations of different subtype sequences as described in U.S. Pat. Nos. 4,414,150, 4,456,748 and 4,678,751. A commercially available consensus interferon is marketed as INFERGEN® (interferon alfacon-1) by Valeant Pharmaceuticals, Costa Mesa, Calif.

Also included within the meaning of “type I interferon” are any of the foregoing molecules that have been covalently modified (referred to herein as a “modified interferon”) to enhance one or more of its pharmacokinetic or pharmacodynamic properties, such as conjugates between a type I interferon and a water soluble polymer and fusions between interferon and a non-interferon protein. A non-limiting list of polymers that may comprise interferon-polymer conjugates useful in practicing the present invention are polyalkylene oxide homopolymers such as polypropylene glycols, polyoxyethylenated polyols, copolymers thereof and block copolymers thereof, dextran polyvinylpyrrolidones, polyacrylamides, polyvinyl alcohols, and carbohydrate-based polymers. Examples of interferon-polymer conjugates are described in U.S. Patent Application Publication No. US 2004/0030101 A1, U.S. Pat. Nos. 6,113,906, 6,042,822, 5,951,974, 5,919,455, 5,738,846, 5,711,944, 5,643,575, 4,917,888 and 4,766,106.

Particularly preferred interferon-polymer conjugates are pegylated interferons, which are conjugates between polyethylene glycol (PEG) and a type I interferon. Commercially available PEGylated versions of IFNα-2 use herein include those sold under the trademarks PEGASYS® (made by Hoffmann La Roche) and PEG-INTRON® (Schering Plough). An exemplary interferon fusion protein is sold under the trademark ALBUFERON®, which is a fusion between human serum albumin (HSA) and IFN-α which was created by Human Genome Sciences, Rockville, Md.

As used herein, the term “pegylated interferon” means a covalent conjugate between at least one PEG moiety and at least one type I interferon molecule. In some embodiments, the PEG moiety consists of a linear PEG chain; while in other embodiments, the PEG moiety has a branched structure. Use of a branched PEG moiety allows attachment of two PEG molecules to the interferon molecule via a single linkage, with the resulting conjugate typically referred to as PEG2-IFN (US 2004/0030101 A1) or U-PEG-IFN (U.S. Pat. No. 6,113,906) or branched-PEG-IFN.

Pegylated interferons may be prepared using a PEG composition having an average molecular weight ranging from about 200 to about 66,000 daltons, with preferred average molecular weights between 2,000 and 45,000 daltons. In describing specific pegylated interferons herein, the average molecular weight of the PEG polymer moiety is designated with a number shown as a subscript following PEG, i.e., PEG n. The conjugation reaction may be performed with a wide variety of commercially available pegylation linkers, which use chemistries that target specific moieties on proteins, such as specific amino acid side chains and the N-terminal amine. One preferred linker chemistry employs N-hydroxysuccinimide (NHS)-PEG, which forms amide bonds with lysine side chain groups and the N-terminus of the interferon. This chemistry is used to make PEGASYS® (interferon alpha 2a, Hoffmann-LaRoche, Nutley, N.J.) (see U S 2004/0030101 A1).

A suitable pegylated interferon for use in the present invention is PEG-Intron® (pegylated interferon α-2b, Schering Corporation), which is manufactured using succinimydyl carbonate (SC)-PEGi 2000- This linker forms urethane bonds between PEGi 2,000 molecules and interferon molecules (see U.S. Pat. No. 5,951,974). Pegylation with SC-PEGi2000 typically produces a mixture of positional isomers of single, linear PEG molecules attached to single interferon molecules at different amino acid residues (See, e.g., Grace et al., Structural and biologic characterization of Pegylated Recombinant IFN-α; 2b, J. Interferon and Cytokine Research 2 1:1103-1115 (2001) When pegylation is performed at mildly acidic conditions as described in U.S. Pat. No. 5,951,974, pegylation at His 34 of IFN-α; -2b is favored (Wylie et al., Carboxylated Histidine Is a pH-Dependent Product of Pegylation with SC-PEG, Pharmaceutical Research 18 (9): 1354-1360 (2001).

The ability of any particular type I interferon, as defined above, to activate the IFNAR may be tested using techniques well-known in the art, such as measuring mRNA or protein levels for genes whose expression is known to be induced by activation of the EFNAR. For example, biomarkers of biologically active type I interferons include IPIO and other IFN-α; inducible proteins, 2′5′ oligoadenylate and neopterin in the plasma, and interferon-gamma in the urine and plasma. Such biomarker expression can also be used as surrogate pharmacodynamic endpoints in determining a dosing regimen for a particular type I interferon to provide interferon plasma levels required for half-maximal binding to the IFNAR in the bloodstream.

### IV. JAK 2 Inhibitors

A Jak2 inhibitor is any compound that selectively inhibits the phosphorylation of the Jak2 protein in the Jak/STAT pathway. The compound may directly inhibit Jak2, or a component upstream of Jak2. The inhibition of the Jak2 protein must be sufficient to substantially inhibit and preferably prevent the Jak/STAT cascade. The Jak2 inhibitor may be any type of compound. For example, the compound may be a small organic molecule or a biological compound, such as an antibody or an enzyme.

Examples of Jak2 inhibitors include INCB018424 (Incyte), TG101348 (TargGen), CEP-701 (lestaurtinib) (Cephalon), AZD1480 (AstraZeneca, XL019 (Exelixis), CYT-387 (Cytopia), SGI-1252 (superGen), SB1518 (S*BIO), tasocitinib (CP-690550), LY3009104 (INCB28050) tyrphostins (see Meydan et al., (1996) Nature, 379:645-648; Levitzki et al, (1995) Science, 267:1782-1788; and PCT application WO 98/06391) including AG490, the inhibitor peptide Tkip, Z3, C7, and TG101209 (Mayo Clinic). Exemplary non specific inhibitors of Jak2 include Go6976, Erlotinib, Atiprimod, CP-690,550, AT9283 and MK-0457.

A compound is considered a selective inhibitor of Jak2 when the compound inhibits Jak2 activity to an extent significantly greater than it inhibits the activity of other members of the Jak family, e.g., Jak1, Jak3, and Tyk2. Preferably, the selective inhibitor inhibits Jak2 at least 2-fold more than it inhibits other members of the Jak family, more preferably at least about 5-fold more, and most preferably at least about 10-fold more.

Methods for screening for compounds that inhibit members of the Jak family are known in the art. For example, a phosphotyrosine assay is described in Example 5 of U.S. Pat. No. 7,235,588. See also Molecular Cloning A Laboratory Manual by J. Sambrook and D. W. Russel, 2001. Jak2 inhibitors as defined herein also include pharmaceutically acceptable salts. As used herein, pharmaceutically acceptable salts may be formed by treating the compounds identified above with salt-forming acids and bases which do not substantially increase the toxicity of the compound.

### EXAMPLES

### Materials and Methods

Tumor Cell Lines and Murine Models.

The B16/F10 murine melanoma cell line was derived from a spontaneous melanoma in the C57BL/6J mouse of the H-2B background and was provided by Dr. Isaiah Fidler (The University of Texas M. D. Anderson Cancer Center [M. D. Anderson], Houston, Tex.). The B16 model system is known for its propensity to develop LMD.²⁶ The B16 cells were maintained in RPMI 1640 medium supplemented with 10% FBS at 37° C. in a humidified atmosphere of 5% CO₂ and 95% air. All cell lines were grown in antibiotic-free medium and were free of Mycoplasma contamination. Coligan J E, Kruisbeck A M, Margulies D H, Shevach E M, Strober W., Current Protocols In Immunologyed, New York: Green & Wiley Interscience, 1994.

For the in vivo experiments, we used 4- to 6-week-old female C57BL/6J mice maintained in the M. D. Anderson Cancer Center Isolation Facility in accordance with Laboratory Animal Resources Commission standards and conducted according to an Institutional Animal Care and Use Committee-approved protocol, 08-06-11831. A mouse was euthanized when it became unable to reach food or water. To induce intracerebral tumors in C57BL/6J mice, B16 cells were collected in logarithmic growth phase, washed twice with PBS, mixed with an equal volume of 10% methyl cellulose and RPMI 1640 medium and loaded into a 250-μl syringe (Hamilton, Reno, Nev.) with an attached 25-gauge needle. The needle was positioned 2 mm to the right of bregma and 4 mm below the surface of the skull at the coronal suture using a stereotactic frame (Kopf Instruments, Tujunga, Calif.). The intracerebral tumorigenic dose for the B16 cells was 5×10² in a total volume of 5 μl.

Immune Therapeutics.

pORF.IFN-α (IFN-α) plasmid was obtained from Invivogen (29). Hydrodynamic gene transfer (HGT) consisted of a single intravenous (i.v.) injection of 3 μg endotoxin-free pORF plasmid encoding murine IFN-α or pORF control plasmid DNA (InvivoGen, San Diego, Calif.) in 2 ml of saline as previously described. Liu F, Song Y, Liu D, Hydrodynamics-Based Transfection In Animals By Systemic Administration Of Plasmid DNA, Gene Therapy 1999; 6:1258-66. This results in the sustained in vivo expression of IFN-α at serum levels of 900 pg/ml for more than 30 days after administration. Sikora A G, Jaffarzad N, Hailemichael Y, Gelbard A, Stonier S W, Schluns K S, Frasca L, Lou Y, Liu C, Andersson H A, Hwu P, Overwijk W W, IFN-A Enhances Peptide Vaccine-Induced CD8+ T Cell Numbers, Effector Function, And Antitumor Activity, J Immunol 2009, 182:7398-407. The STAT3 inhibitor, WP1193, was synthesized and supplied by Dr. Priebe (M. D. Anderson). WP1193, a third-generation analogue inhibitor of the p-STAT3 pathway, was dissolved in a mixture of 20 parts dimethylsulfoxide (DMSO) to 80 parts polyethylene glycol (PEG) 300 (Sigma-Aldrich, St Louis, Mo.) at titered concentrations and delivered in a final volume of 100 μL. Prior to use, WP1193 was stored as a lyophilized powder at 4° C.

Treatment for the established intracerebral tumors was with WP1193 (starting on day 3) and/or IFN-α (starting on day 5) after B16 tumor cell challenge. Mice were injected intravenously (i.v.) with 2 ml (3 μg) of IFN-α plasmid in saline once. Mice were treated with a sub-therapeutic dose of 30 mg/kg of WP1193 by oral gavage (o.g.) in a vehicle of DMSO/PEG300 (20 parts/80 parts) on Monday, Wednesdays and Fridays, on a q.i.d. schedule (5 days on, 2 days off). When mice were treated in the therapeutic range of 40 mg/kg, >80% of animals survived long-term (more than 70 days) and synergy with IFN-α could not be assessed. Ten mice per experimental group were used, including treatment with the DMSO/PEG300 vehicle alone in the control group.

Immunoblotting analysis. Murine melanoma B16 cells and splenocytes were used for protein isolation and immunoblotting analysis as described below. B16 cells were seeded at a density of 2×10⁶ cells/well in 6-well culture plates and incubated at 37° C., in an atmosphere containing 5% CO₂, with the RPMI 1640 medium overnight. Afterward, B16 cells were cultured in the absence or presence of WP1193 (5 μM, 10 μM). After 3.5 hours, the B16 cells were further cultured in the absence or presence of 20 ng/ml of IFN-α for 30 minutes. For the splenocyte preparation, spleens from two 4- to 6-week-old female mice were harvested and disassociated into a single cell suspension. After erythrocytes were lysed with 1×RBC lysis buffer (eBioscience, San Diego, Calif.), splenocytes were washed once with RPMI 1640 medium and were seeded at a density of 10×10⁶ cells/well in 6-well culture plates and incubated at 37° C., in an atmosphere containing 5% CO₂, with the RPMI 1640 medium in absence or presence of WP1193 (5 μM, 10 μM). After 1.5 hours, the splenocytes were further cultured in the absence or presence of 20 ng/ml of IFN-α for 30 minutes. Afterwards, all the above B16 cells and splenocytes were pelleted and rinsed with ice-cold PBS at 1500 rpm for 5 minutes. The cells were lysed for 30 minutes in ice-cold lysis buffer (50 mM Tris-HCl [pH 8.0], 150 mM NaCl, 1 mM EDTA) containing 1% Triton-X-100 and phosphatase and protease inhibitors (Sigma-Aldrich). The lysates were centrifuged at 14,000 rpm for 10 minutes at 4° C. The supernatants were collected and quantified for protein content. Equal amounts of proteins (65 μg) were electrophoretically fractionated in 8% sodium dodecyl sulfate (SDS)-polyacrylamide gels, transferred to nitrocellulose membranes, and subjected to immunoblot analysis with specific antibodies against p-STAT3 (Tyr705), STAT3 (Cell Signaling Technology, Inc., Danvers, Mass.), and β-actin (Sigma-Aldrich).

Autoradiography of the membranes was performed using Amersham ECL Western blotting detection reagents (Amersham Biosciences). The densities of the protein bands compared with the β-actin protein control were measured with the Image J program provided by the NIH.

Determination of serum cytokine levels. Groups of non-tumor bearing 4- to 6-week-old female mice were treated with either IFN-α or “empty” plasmid control, WP1193, or IFN-α in combination with WP1193 as described in FIG. 13. Blood was collected through a tail vein from mice 9 and 16 days after the first WP1193 administration. The serum was separated and stored at −20° C. for the measurement of IL-1β, IL-2, IL-4, IL-10, IL-12, TNF-α and IFN-α concentrations using the Bio-Plex Pro mouse cytokine 8-plex assay kit (Bio-Rad, Hercules, Calif.). The assay was run according to the manufacturer's instruction (Bio-Rad) by the Immunology Core Service at MD Anderson Cancer Center (Houston, Tex.). Sensitivity of the assay for IL-β, IL-2, IL-4, IL-10, IL-12, TNF-α or IFN-α is 9.4, 0.6, 2.1, 1.0, 2.3, 1.4, and 1.2 pg/ml, respectively.

Determination of inhibition of Tregs in vivo. To ascertain the inhibition of Tregs within the bone marrow, lymph node, spleen, thymus and peripheral blood compartment, non-tumor bearing mice were treated with IFN-α or “empty” plasmid control, WP1193, or IFN-α in combination with WP1193 for 9 and 16 days as described in the above schema. Single-cell suspensions were prepared from bone marrows, lymph nodes, spleens, thymuses and peripheral blood of mice. Single cells were surface-stained by FITC-conjugated anti-CD4 (L3T4), PerCP-conjugated anti-CD8 (53-6.7) and APC-conjugated anti-CD25 (PC61), and the cells were further subjected to intracellular staining with PE-conjugated mAbs to mouse FoxP3 (clone FJK-16s; eBioscience, San Diego, Calif.) using staining buffers and conditions specified by the manufacturer.

Ex Vivo Splenocytes Cytotoxicity Assays.

Spleens from 4- to 6-week-old female mice in the above treatment schema were harvested and disassociated into a single cell suspension. After erythrocytes were lysed with 1×RBC lysis buffer (eBioscience), splenocytes were washed once with RPMI 1640 medium and were ready as effector cells for the standard cytotoxicity assay. Wang L, Yi T, Kortylewski M, Pardoll D M, Zeng D, Yu H, IL-17 Can Promote Tumor Growth Through An IL-6-Stat3 Signaling Pathway, J Exp Med 2009; 206:1457-64. For target cells, B16 cells in RPMI 1640 medium were cultured for 3 days, trypsinized, pelleted, and resuspended in FACS buffer at room temperature to achieve a concentration of 10⁶ cells/ml. Carboxy-fluorescein diacetate succinimidyl ester (CFSE) stock solution (CellTrace CFSE Cell Proliferation Kit; Invitrogen, Eugene, Oreg.) was added to achieve a final concentration of 4 μM. The mixture was incubated at 37° C. for 10 minutes, and then the staining reaction was quenched by the addition of five volumes of ice-cold PBS for 5 minutes. The B16 cells were washed three times in RPMI 1640 medium and plated for the cytotoxicity assay. The ratios of splenocyte effector cells to B16 target cells were 30:1 and 100:1. After 48 h of incubation, the CFSE-labeled B16 melanoma cells were removed from the plates with trypsin-EDTA (0.05%) and analyzed by FACS. The B16 cells were stained with propidium iodide (PI; BD Biosciences) to distinguish viable cells from nonviable cells. B16 cells that were stained with CFSE and PI were considered nonviable. Flow cytometric acquisition of the B16 target cells was performed with a FACSCalibur flow cytometer (BD Biosciences), and data analysis was performed using FlowJo software (TreeStar, Ashland, Oreg.).

NK Cell and T Cell Cytotoxicity Assay Against Melanoma Cells.

Splenocytes were prepared as described above. NK1.1+CD3− NK effector cells or CD3+CD8+ T effector cells were sorted from splenocytes on a FACSAria Cell Sorter (BD Biosciences) with FITC-conjugated anti-mouse NK1.1 (eBioscience), PE-conjugated anti-CD3, and allophycocyanin (APC)-conjugated anti-CD8 antibodies (Miltenyi Biotec, Auburn, Calif.). B16 target cells were prepared as described above. The ratio of NK1.1+CD3− NK effector cells or CD8+ T effector cells to B16 target cells was 10:1 and 5:1, respectively. In the presence of NK1.1+CD3− NK cells or CD8+ T cells, treatment groups consisted of B16 target cells alone; B16 cells with 2 μM of WP1193; B16 cells with 20 ng/ml of IFN-α; B16 cells with 2 □M of WP1193 and 20 ng/ml of IFN-α. After 48 hours of incubation, the CFSE-labeled B16 melanoma cells were removed from the plates with trypsin and analyzed by FACS. Then, B16 cells were stained with PI (BD Biosciences) to distinguish viable cells from nonviable cells. B16 cells that were stained with CFSE and PI were considered nonviable. Flow cytometric acquisition of the B16 target cells was performed with a FACSCalibur flow cytometer (BD Biosciences), and data analysis was performed using FlowJo software (TreeStar).

Detection of NK Cell Receptors.

Spleens from 4- to 6-week-old female mice were harvested and disassociated into a single-cell suspension as described above. Splenocytes were seeded at a density of 4×10⁶ cells/well in 24-well culture plates and cultured with the RPMI 1640 medium in absence or presence of WP1193 (2 μM) and/or INF-α (20 ng/ml) for 24 hours. Afterwards, the cells were harvested and washed twice in PBS with 5% FCS, resuspended in staining buffer and labeled with FITC- or PE-conjugated anti-mouse NK1.1 (eBioscience, San Diego, Calif.) to identify the NK population. To stain in duplicate, 10⁶ cells were transferred to a 96-well plates and Fc staining was blocked with rat anti-mouse CD16/CD32 serum (BD Biosciences) and the cells were incubated for 15 minutes at room temperature. Secondary staining was performed with FITC-conjugated rat anti-mouse CD94 (KLRD1) mAb (Lifespan Biosciences, Seattle, Wash.), biotin-conjugated rat anti-mouse NKG2C mAb (AbD Serotec; Raleigh, N.C.), PE-conjugated rat anti-mouse NKG2D (CD314) mAb (Biolegend, San Diego, Calif.), FITC-conjugated rat anti-mouse NKp46 (NCR1) mAb (R&D Systems, Minneapolis, Minn.), or Alexa Fluor 647-conjugated rat anti-mouse CD226 (DNAM-1) mAb (Biolegend). Negative control wells were stained with the corresponding isotypes. Following incubation, the cells were washed twice with FACS buffer and then analyzed with a BD FACSCalibur with gates set for viable splenocytes.

Detection of MHC Class I (MHC I), MHC Class II (MHC II), and NK Cell Ligands on B16 Melanoma Cells.

To ascertain the MHC I, MHC II, and NK ligands expressed on melanoma cells, B16 cells were seeded at a density of 2×10⁶ cells/well in 24-well culture plates and cultured with the RPMI 1640 medium in the absence or presence of WP1193 (2 μM) and/or INF-α (20 ng/ml) for 24 hours. Afterwards, the cells were harvested and washed twice, and 10⁶ cells in duplicate were Fc blocked with purified rat anti-mouse CD16/CD32 (BD Biosciences) for 15 minutes at room temperature. The B16 cells were washed and then stained for approximately 30 minutes at 4° C. with FITC-conjugated rat anti-mouse MHC I mAb (Abeam, Cambridge, Mass.), PE-conjugated rat anti-mouse MHC Class II mAb (Abeam), FITC-conjugated rat anti-mouse Rae-1 mAb (R&D Systems), APC-conjugated rat anti-mouse H60 mAb (R&D Systems) or PE-conjugated rat anti-mouse CD155 mAb (Biolegend). Negative control cells were stained with the corresponding isotypes. Following incubation, the cells were washed twice with FACS buffer and then analyzed with a BD FACSCalibur with gates set for viable cells.

Statistics.

Kaplan-Meier product-limit survival probability estimates of overall survival were calculated and log-rank tests were performed to compare overall survival between treatment groups and the control arm. Kaplan E L, Meier P., Nonparametric Estimation From Incomplete Observations, J Am Stat Assoc 1958; 53:457-81; Mantel N., Evaluation Of Survival Data And Two New Rank Order Statistics Arising In Its Consideration, Cancer Chemother Rep 1966; 50:163-70 Ex-vivo or in vitro data are presented as means±standard errors (SEs) of three repeated experiments. Student's t test was performed. A P value below 0.05 was considered statistically significant.

Synthetic Chemistry.

The compounds disclosed herein can be prepared by following the procedures described in WO 2005058829 (pages 22-29), US 20050277680, U.S. Pat. No. 7,745,468 (column 20, line 1 to column 25, line 12), WO 2007115269 (pages 40-52), US 20070232668 (pages 17-22), and WO 2010005807 (paragraphs [0191]-[0201]), each of which is hereby incorporated by reference in their entirety; methods known to one of skill in the art; and routine modifications thereof. The invention is further illustrated by the following examples. All IUPAC names were generated using CambridgeSoft's ChemDraw 11.0.

Results

Using molecular modeling and medicinal chemistry approaches, we designed and developed a panel of unique small molecule inhibitors that block STAT3 phosphorylation, in vitro and in vivo, based on the caffeic acid benzyl ester/AG490 scaffold (Examples 1 to 63)., Madden T, Kazerooni R, Myer J, Culotta K, Donato N, Johansen M, Kondo Y, Mack D, Priebe W., The Preclinical Pharmacology Of WP1066, A Potent Small Molecule Inhibitor Of The JAK2/STAT3 Pathway, 97th Annual Meeting of the American Association for Cancer Research Washington, D.C., 2006; Kim K Y, Kim J K, Han S H, Lim J S, Kim K I, Cho D H, Lee M S, Lee J H, Yoon D Y, Yoon S R, Chung J W, Choi I, et al., Adiponectin Is A Negative Regulator Of NK Cell Cytotoxicity, J Immunol 2006; 176:5958-64. WP1193 is a third-generation analogue that has an additional aromatic ring on the benzyl amine moiety and was selected for these studies based on its potential to be a potent immune modulator.

As shown in FIGS. 3A, and 3B, WP1193 inhibits the phosphorylation of p-STAT3 in both B16 cells (FIG. 3A) and in splenocytes (FIG. 3B). In this experiment, B16 cells and splenocytes isolated from C57BL/6J mice and were incubated with either the medium, medium supplemented with titrated WP1193, medium supplemented with IFN-α, or medium supplemented with both IFN-α and WP1193. After 2 hours (splenocytes) or 4 hours (B16 cells), cells were lysed, electrophoretically fractionated in 8% SDS-polyacrylamide gels, transferred to nitrocellulose membranes, and immunoblotted with antibodies to p-STAT3, total STAT3 and β-actin. Semi-quantitative densitometry was used to determine the relative levels of p-STAT3 to STAT3 and β-actin.

### Example 1

### (S,E)-3-(6-bromopyridin-2-yl)-2-cyano-N-(1-phenylethyl)acrylamide (WP1066)

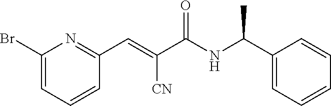 [SMILES: C[C@H](NC(=O)/C(C#N)=C/c1cccc(Br)n1)c1ccccc1]

### Example 2

### (E)-N-benzyl-3-(6-bromopyridin-2-yl)-2-cyanoacrylamide (WP1015)

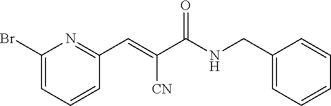 [SMILES: N#C/C(=C\c1cccc(Br)n1)C(=O)NCc1ccccc1]

### Example 3

### (S,E)-3-(6-bromopyridin-2-yl)-2-cyano-N-(1-phenylbutyl)acrylamide (WP1130)

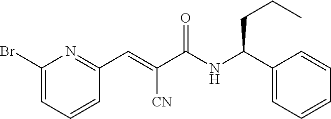 [SMILES: CCC[C@H](NC(=O)/C(C#N)=C/c1cccc(Br)n1)c1ccccc1]

### Example 4

### (S,E)-3-(6-bromopyridin-2-yl)-2-cyano-N-(1-phenylpropyl)acrylamide (WP1129)

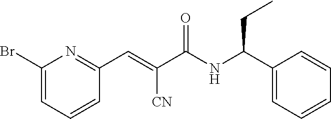 [SMILES: CC[C@H](NC(=O)/C(C#N)=C/c1cccc(Br)n1)c1ccccc1]

### Example 5

### (E)-N-benzyl-2-cyano-3-(4-nitrophenyl)acrylamide (AG1801)

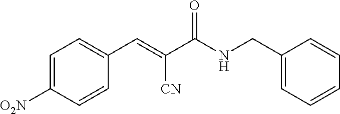 [SMILES: N#C/C(=C\c1ccc([N+](=O)[O-])cc1)C(=O)NCc1ccccc1]

### Example 6

### (R,E)-2-cyano-3-(4-nitrophenyl)-N-(1-phenylethyl)acrylamide (WP1034)

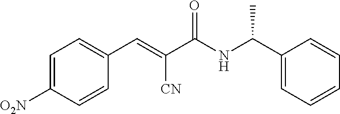 [SMILES: C[C@@H](NC(=O)/C(C#N)=C/c1ccc([N+](=O)[O-])cc1)c1ccccc1]

### Example 7

### (R,E)-2-cyano-3-(3-hydroxy-4-nitrophenyl)-N-(1-phenylethyl)acrylamide (WP1038)

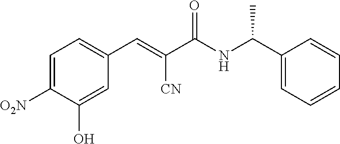 [SMILES: C[C@@H](NC(=O)/C(C#N)=C/c1ccc([N+](=O)[O-])c(O)c1)c1ccccc1]

### Example 8

### (S,E)-2-cyano-3-(4-nitrophenyl)-N-(1-phenylethyl)acrylamide (WP1050)

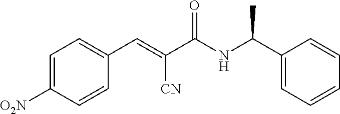 [SMILES: C[C@H](NC(=O)/C(C#N)=C/c1ccc([N+](=O)[O-])cc1)c1ccccc1]

### Example 9

### (S,E)-2-cyano-3-(3-hydroxy-4-nitrophenyl)-N-(1-phenylethyl)acrylamide (WP1051)

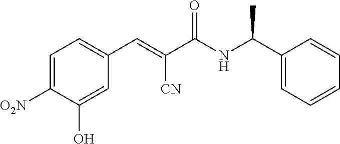 [SMILES: C[C@H](NC(=O)/C(C#N)=C/c1ccc([N+](=O)[O-])c(O)c1)c1ccccc1]

### Example 10

### (S,E)-2-cyano-N-(1-phenylethyl)-3-(pyridin-2-yl)acrylamide (WP1065)

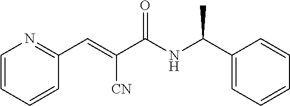 [SMILES: C[C@H](NC(=O)/C(C#N)=C/c1ccccn1)c1ccccc1]

### Example 11

### (E)-N-benzyl-2-cyano-3-(pyridin-3-yl)acrylamide (WP1075)

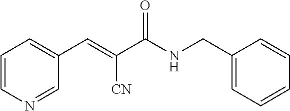 [SMILES: N#C/C(=C\c1cccnc1)C(=O)NCc1ccccc1]

### Example 12

### (E)-N-benzyl-3-(4-chloro-3-nitrophenyl)-2-cyanoacrylamide (WP1077)

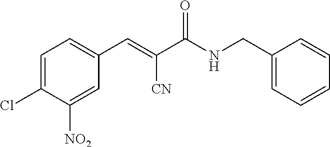 [SMILES: N#C/C(=C\c1ccc(Cl)c([N+](=O)[O-])c1)C(=O)NCc1ccccc1]

### Example 13

### (S,E)-cyclopropyl(phenyl)methyl 3-(6-bromopyridin-2-yl)-2-cyanoacrylate (WP1332)

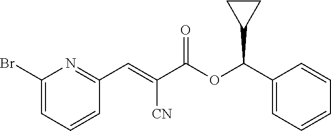 [SMILES: N#C/C(=C\c1cccc(Br)n1)C(=O)O[C@H](c1ccccc1)C1CC1]

### Example 14

### (S,E)-2-cyano-N-(cyclopropyl(phenyl)methyl)-3-(3,4-dihydroxyphenyl)acrylamide (WP1331)

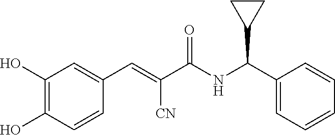 [SMILES: N#C/C(=C\c1ccc(O)c(O)c1)C(=O)N[C@H](c1ccccc1)C1CC1]

### Example 15

### (S,E)-cyclopropyl(phenyl)methyl 2-cyano-3-(3,4-dihydroxyphenyl)acrylate (WP1330)

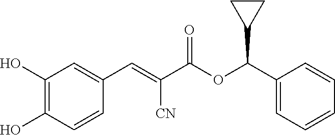 [SMILES: N#C/C(=C\c1ccc(O)c(O)c1)C(=O)O[C@H](c1ccccc1)C1CC1]

### Example 16

### (S,E)-cyclopropyl(phenyl)methyl 3-(3,4-dihydroxyphenyl)acrylate (WP1329)

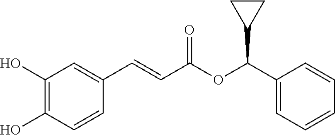 [SMILES: O=C(/C=C/c1ccc(O)c(O)c1)O[C@H](c1ccccc1)C1CC1]

### Example 17

### (S,E)-N-(cyclopropyl(phenyl)methyl)-3-(3,4-dihydroxyphenyl)acrylamide (WP1328)

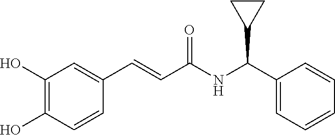 [SMILES: O=C(/C=C/c1ccc(O)c(O)c1)N[C@H](c1ccccc1)C1CC1]

### Example 18

### (R,E)-2-(3-(6-bromopyridin-2-yl)-2-cyanoacrylamido)-2-phenylethyl pivalate (WP1302)

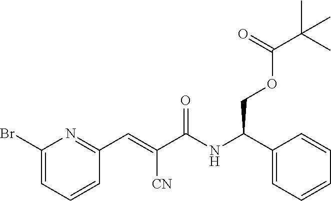 [SMILES: CC(C)(C)C(=O)OC[C@H](NC(=O)/C(C#N)=C/c1cccc(Br)n1)c1ccccc1]

### Example 19

### (R,E)-2-(3-(6-bromopyridin-2-yl)-2-cyanoacrylamido)-2-phenylethyl acetate (WP1293)

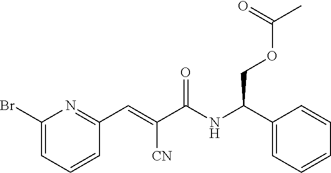 [SMILES: CC(=O)OC[C@H](NC(=O)/C(C#N)=C/c1cccc(Br)n1)c1ccccc1]

### Example 20

### (S,E)-3-(6-bromopyridin-3-yl)-2-cyano-N-(1-phenylethyl)acrylamide (WP1286)

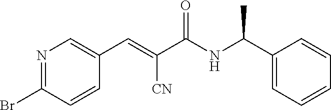 [SMILES: C[C@H](NC(=O)/C(C#N)=C/c1ccc(Br)nc1)c1ccccc1]

### Example 21

### (S,E)-3-(6-bromopyridin-2-yl)-N-(1-phenylethyl)acrylamide (WP1204)

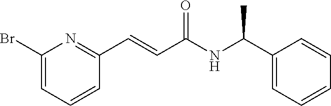 [SMILES: C[C@H](NC(=O)/C=C/c1cccc(Br)n1)c1ccccc1]

### Example 22

### (S,E)-3-(2-bromopyridin-3-yl)-2-cyano-N-(1-phenylethyl)acrylamide (WP1285)

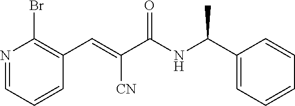 [SMILES: C[C@H](NC(=O)/C(C#N)=C/c1cccnc1Br)c1ccccc1]

### Example 23

### (S,E)-3-(5-bromopyridin-3-yl)-2-cyano-N-(1-phenylethyl)acrylamide (WP1284)

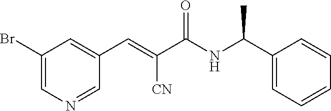 [SMILES: C[C@H](NC(=O)/C(C#N)=C/c1cncc(Br)c1)c1ccccc1]

### Example 24

### (S,E)-3-(3-bromopyridin-4-yl)-2-cyano-N-(1-phenylethyl)acrylamide (WP1283)

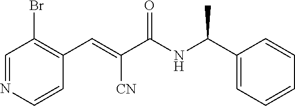 [SMILES: C[C@H](NC(=O)/C(C#N)=C/c1ccncc1Br)c1ccccc1]

### Example 25

### (S,E)-3-(6-bromopyridin-2-yl)-2-cyano-N-(cyclopentyl(phenyl)methyl)acrylamide (WP1282)

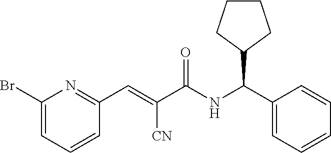 [SMILES: N#C/C(=C\c1cccc(Br)n1)C(=O)N[C@H](c1ccccc1)C1CCCC1]

### Example 26

### (S,E)-2-cyano-3-(3-fluoropyridin-4-yl)-N-(1-phenylethyl)acrylamide (WP1280)

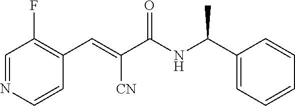 [SMILES: C[C@H](NC(=O)/C(C#N)=C/c1ccncc1F)c1ccccc1]

### Example 27

### ((S,E)-2-cyano-3-(2-methoxypyridin-3-yl)-N-(1-phenylethyl)acrylamide (WP1273)

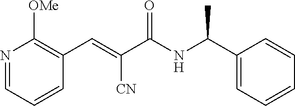 [SMILES: COc1ncccc1/C=C(\C#N)C(=O)N[C@@H](C)c1ccccc1]

### Example 28

### (S,E)-2-cyano-N-(cyclopropyl(phenyl)methyl)-3-(2-fluoropyridin-3-yl)acrylamide (WP1272)

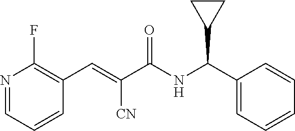 [SMILES: N#C/C(=C\c1cccnc1F)C(=O)N[C@H](c1ccccc1)C1CC1]

### Example 29

### (S,E)-(6-(2-cyano-3-(cyclopropyl(phenyl)methylamino)-3-oxoprop-1-enyl)pyridin-2-yl)methyl acetate (WP1246)

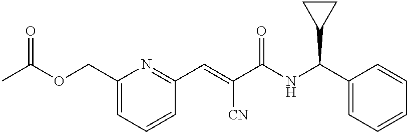 [SMILES: CC(=O)OCc1cccc(/C=C(\C#N)C(=O)N[C@H](c2ccccc2)C2CC2)n1]

### Example 30

### (S,E)-3-(6-chloropyridin-2-yl)-2-cyano-N-(cyclopropyl(phenyl)methyl)acrylamide (WP1229)

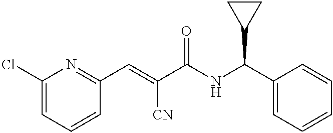 [SMILES: N#C/C(=C\c1cccc(Cl)n1)C(=O)N[C@H](c1ccccc1)C1CC1]

### Example 31

### (R,E)-3-(6-bromopyridin-2-yl)-2-cyano-N-(2-hydroxy-1-phenylethyl)acrylamide (WP1269)

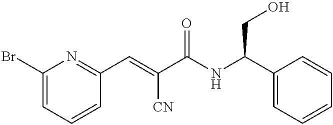 [SMILES: N#C/C(=C\c1cccc(Br)n1)C(=O)N[C@@H](CO)c1ccccc1]

### Example 32

### (S,E)-2-cyano-3-(2-fluoropyridin-3-yl)-N-(1-phenylethyl)acrylamide (WP1271)

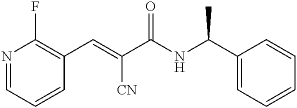 [SMILES: C[C@H](NC(=O)/C(C#N)=C/c1cccnc1F)c1ccccc1]

### Example 33

### (S,E)-3-(6-bromopyridin-2-yl)-2-cyano-N-(1-hydroxy-3-phenylpropan-2-yl)acrylamide (WP1268)

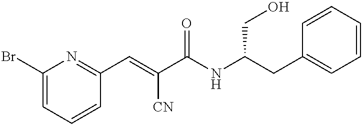 [SMILES: N#C/C(=C\c1cccc(Br)n1)C(=O)N[C@H](CO)Cc1ccccc1]

### Example 34

### (S,E)-3-(6-bromopyridin-2-yl)-2-cyano-N-(cyclobutyl(phenyl)methyl)acrylamide (WP1267)

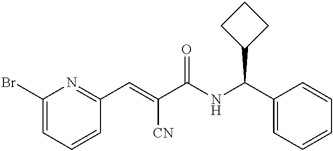 [SMILES: N#C/C(=C\c1cccc(Br)n1)C(=O)N[C@H](c1ccccc1)C1CCC1]

### Example 35

### (S,E)-2-cyano-3-cyclododecyl-N-(1-phenylethyl)acrylamide (WP1203)

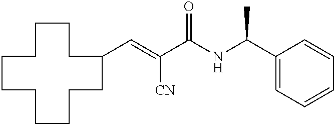 [SMILES: C[C@H](NC(=O)/C(C#N)=C/C1CCCCCCCCCCC1)c1ccccc1]

### Example 36

### (S,E)-2-(6-bromopyridin-2-yl)-N-(1-phenylethyl)ethenesulfonamide (WP1201)

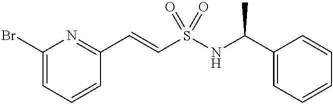 [SMILES: C[C@H](NS(=O)(=O)/C=C/c1cccc(Br)n1)c1ccccc1]

### Example 37

### (S,E)-2-cyano-3-(1H-imidazol-2-yl)-N-(1-phenylethyl)acrylamide (WP1196)

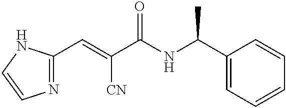 [SMILES: C[C@H](NC(=O)/C(C#N)=C/c1ncc[nH]1)c1ccccc1]

### Example 38

### (R,E)-2-cyano-3-(6-methylpyridin-2-yl)-N-(1-phenylethyl)acrylamide (WP1180)

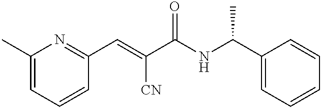 [SMILES: Cc1cccc(/C=C(\C#N)C(=O)N[C@H](C)c2ccccc2)n1]

### Example 39

### (S,E)-2-cyano-3-(6-methylpyridin-2-yl)-N-(1-phenylethyl)acrylamide (WP1179)

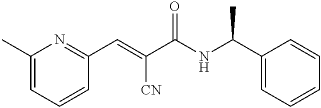 [SMILES: Cc1cccc(/C=C(\C#N)C(=O)N[C@@H](C)c2ccccc2)n1]

### Example 40

### (E)-N-benzhydryl-3-(6-bromopyridin-2-yl)-2-cyanoacrylamide (WP1169)

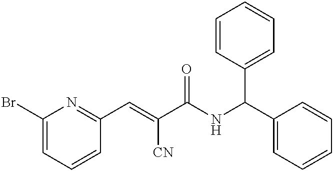 [SMILES: N#C/C(=C\c1cccc(Br)n1)C(=O)NC(c1ccccc1)c1ccccc1]

### Example 41

### (E)-3-(6-bromopyridin-2-yl)-2-cyano-N-((1R,2S)-2-hydroxy-2,3-dihydro-1H-inden-1-yl)acrylamide (WP1168)

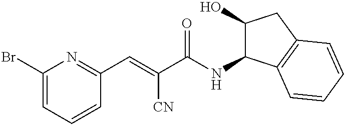 [SMILES: N#C/C(=C\c1cccc(Br)n1)C(=O)N[C@@H]1c2ccccc2C[C@@H]1O]

### Example 42

### (E)-3-(6-bromopyridin-2-yl)-2-cyano-N-((1S,2R)-2-hydroxy-2,3-dihydro-1H-inden-1-yl)acrylamide (WP1167)

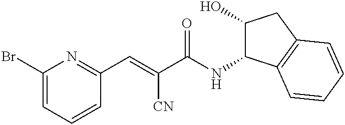 [SMILES: N#C/C(=C\c1cccc(Br)n1)C(=O)N[C@H]1c2ccccc2C[C@H]1O]

### Example 43

### (S,E)-3-(6-bromopyridin-2-yl)-2-cyano-N-(cyclohexyl(phenyl)methyl)acrylamide (WP1166)

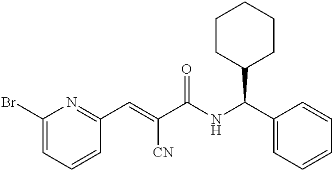 [SMILES: N#C/C(=C\c1cccc(Br)n1)C(=O)N[C@H](c1ccccc1)C1CCCCC1]

### Example 44

### (S,E)-3-(6-bromopyridin-2-yl)-2-cyano-N-(cyclobutyl(phenyl)methyl)acrylamide (WP1164)

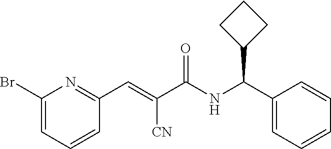 [SMILES: N#C/C(=C\c1cccc(Br)n1)C(=O)N[C@H](c1ccccc1)C1CCC1]

### Example 45

### (R,E)-3-(6-bromopyridin-2-yl)-2-cyano-N-(cyclopropyl(phenyl)methyl)acrylamide (WP1163)

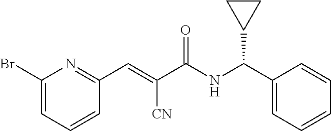 [SMILES: N#C/C(=C\c1cccc(Br)n1)C(=O)N[C@@H](c1ccccc1)C1CC1]

### Example 46

### (E)-3-(6-bromopyridin-2-yl)-2-cyano-N-(2-phenoxyethyl)acrylamide (WP1159)

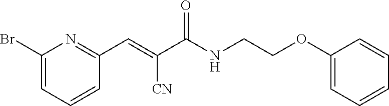 [SMILES: N#C/C(=C\c1cccc(Br)n1)C(=O)NCCOc1ccccc1]

### Example 47

### (S,E)-3-(6-bromopyridin-2-yl)-2-cyano-N-(1,2-diphenylethyl)acrylamide (WP1145)

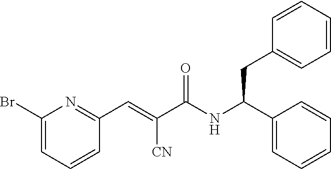 [SMILES: N#C/C(=C\c1cccc(Br)n1)C(=O)N[C@@H](Cc1ccccc1)c1ccccc1]

### Example 48

### (S,E)-3-(6-bromopyridin-2-yl)-2-cyano-N-(cyclopropyl(phenyl)methyl)acrylamide (WP1193)

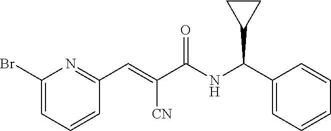 [SMILES: N#C/C(=C\c1cccc(Br)n1)C(=O)N[C@H](c1ccccc1)C1CC1]

### Example 49

### (E)-N-benzyl-2-cyano-3-(cyclohex-3-enyl)acrylamide (WP1082)

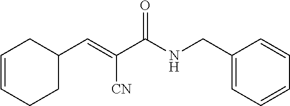 [SMILES: N#C/C(=C\C1CC=CCC1)C(=O)NCc1ccccc1]

### Example 50

### (2E,4E)-5-(6-bromopyridin-2-yl)-2-cyano-N—((S)-1-phenylethyl)penta-2,4-dienamide (WP1220)

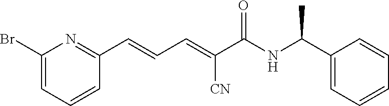 [SMILES: C[C@H](NC(=O)/C(C#N)=C/C=C/c1cccc(Br)n1)c1ccccc1]

### Example 51

### (2E,4E)-5-(6-chloropyridin-2-yl)-2-cyano-N—((S)-1-phenylethyl)penta-2,4-dienamide

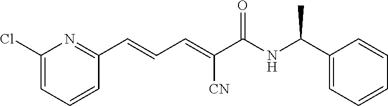 [SMILES: C[C@H](NC(=O)/C(C#N)=C/C=C/c1cccc(Cl)n1)c1ccccc1]

### Example 52

### (2E,4E)-5-(6-bromopyridin-2-yl)-2-cyano-N—((R)-1-phenylethyl)penta-2,4-dienamide

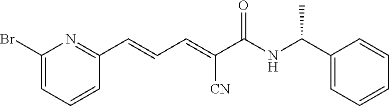 [SMILES: C[C@@H](NC(=O)/C(C#N)=C/C=C/c1cccc(Br)n1)c1ccccc1]

### Example 53

### (2E,4E)-N-benzyl-5-(6-bromopyridin-2-yl)-2-cyanopenta-2,4-dienamide

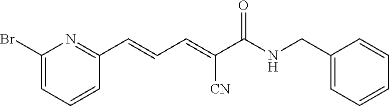 [SMILES: N#C/C(=C\C=C\c1cccc(Br)n1)C(=O)NCc1ccccc1]

### Example 54

### (2E,4E)-5-(6-chloropyridin-2-yl)-2-cyano-N—((R)-1-phenylethyl)penta-2,4-dienamide

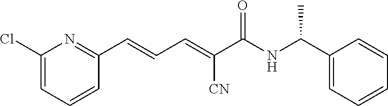 [SMILES: C[C@@H](NC(=O)/C(C#N)=C/C=C/c1cccc(Cl)n1)c1ccccc1]

### Example 55

### (2E,4E)-N-benzyl-5-(6-chloropyridin-2-yl)-2-cyanopenta-2,4-dienamide

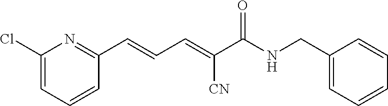 [SMILES: N#C/C(=C\C=C\c1cccc(Cl)n1)C(=O)NCc1ccccc1]

### Example 56

### (2E,4E)-5-(6-bromopyridin-2-yl)-2-cyano-N-((S)-cyclopropyl(phenyl)methyl)penta-2,4-dienamide

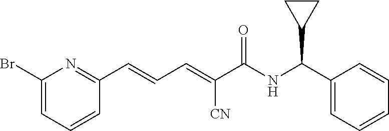 [SMILES: N#C/C(=C\C=C\c1cccc(Br)n1)C(=O)N[C@H](c1ccccc1)C1CC1]

### Example 57

### (2E,4E)-5-(6-bromopyridin-2-yl)-2-cyano-N—((S)-1-phenylpropyl)penta-2,4-dienamide

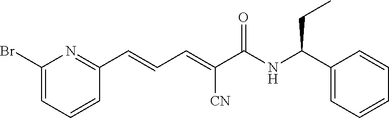 [SMILES: CC[C@H](NC(=O)/C(C#N)=C/C=C/c1cccc(Br)n1)c1ccccc1]

### Example 58

### (2E,4E)-5-(6-bromopyridin-2-yl)-2-cyano-N—((S)-1-phenylbutyl)penta-2,4-dienamide

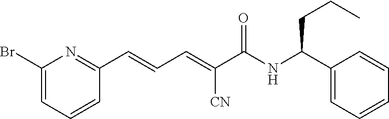 [SMILES: CCC[C@H](NC(=O)/C(C#N)=C/C=C/c1cccc(Br)n1)c1ccccc1]

### Example 59

### (E)-N-benzyl-2-cyano-3-(3,4-dihydroxyphenyl)acrylamide (AG490)

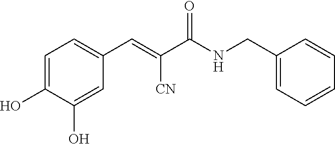 [SMILES: N#C/C(=C\c1ccc(O)c(O)c1)C(=O)NCc1ccccc1]

### Example 60

### (E)-N-benzyl-2-cyano-3-(5-hydroxy-2-nitrophenyl)acrylamide (WP1073)

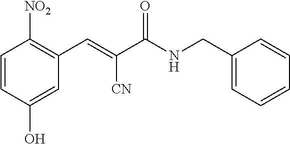 [SMILES: N#C/C(=C\c1cc(O)ccc1[N+](=O)[O-])C(=O)NCc1ccccc1]

### Example 61

### (E)-N-benzyl-2-cyano-3-(3-nitrophenyl)acrylamide (WP1074)

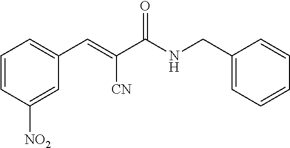 [SMILES: N#C/C(=C\c1cccc([N+](=O)[O-])c1)C(=O)NCc1ccccc1]

### Example 62

### (E)-N-benzyl-2-cyano-3-(4-(dimethylamino)-2-nitrophenyl)acrylamide (WP1076)

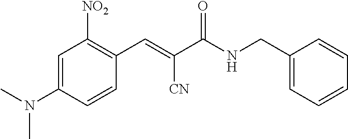 [SMILES: CN(C)c1ccc(/C=C(\C#N)C(=O)NCc2ccccc2)c([N+](=O)[O-])c1]

### Example 63

### (E)-3-(6-bromopyridin-2-yl)-2-cyano-N-(((2R,3R,4S,5R,6S)-3,4,5,6-tetrahydroxytetrahydro-2H-pyran-2-yl)methyl)acrylamide (WP1126)

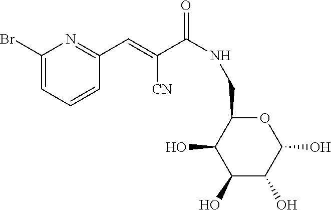 [SMILES: N#C/C(=C\c1cccc(Br)n1)C(=O)NC[C@H]1O[C@H](O)[C@H](O)[C@@H](O)[C@H]1O]

### Example 63

### (E)-3-(6-bromopyridin-2-yl)-2-cyano-N-(((3aR,5R,5aS,8aS,8bR)-2,2,7,7-tetramethyltetrahydro-3aH-bis[1,3]dioxolo[4,5-b:4′,5′-d]pyran-5-yl)methyl)acrylamide (WP1119)

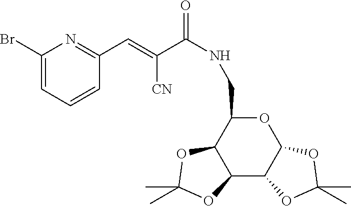 [SMILES: CC1(C)O[C@H]2[C@@H](O1)[C@@H](CNC(=O)/C(C#N)=C/c1cccc(Br)n1)O[C@@H]1OC(C)(C)O[C@@H]12]

### Example 63

### (2R,3R,4S,5S,6R)-6-(((E)-3-(6-bromopyridin-2-yl)-2-cyanoacrylamido)methyl)tetrahydro-2H-pyran-2,3,4,5-tetrayl tetraacetate (WP1127)

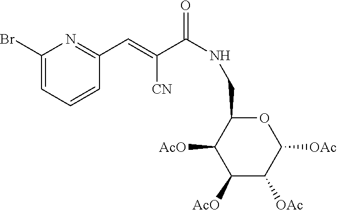 [SMILES: CC(=O)O[C@H]1O[C@H](CNC(=O)/C(C#N)=C/c2cccc(Br)n2)[C@H](OC(C)=O)[C@H](OC(C)=O)[C@H]1OC(C)=O]

### Example 64

### Treatment of Established Intracerebral Melanoma with Both IFN-α and STAT3 Blockade is Efficacious

To determine whether the IFN-α and STAT3 blockade combination therapy yielded a synergistic efficacy against established CNS tumors, IFN-α (i.v.) or WP1193 (o.g.) was administrated alone or in conjunction with each other to C57BL/6J mice in which intracerebral melanoma had been established via intracranial injection of log-phase B16 tumor cells. In order to observe the synergistic effect of STAT3 blockade and IFN-α, the sub-therapeutic dose of WP1193, 30 mg/kg was used in this study. Kaplan-Meier survival curves were plotted for those mice. Upon death, the etiology was confirmed to be tumor progression.

FIG. 4 provides the survival data from C57BL/6J mice treated with WP1193, IFN-α, or both after B16 cells were established in the brain. Median overall survival for tumor-bearing mice without further intervention was 17 days (95% CI, 16, NA; n=11) and was significantly enhanced by all therapies, including with either sub-therapeutic WP1193 (18.5 days; 95% CI, 17, NA; P<0.04 compared with control; n=12) or IFN-α alone (27.5 days; 95% CI, 21, NA; P<0.01 compared with control; n=8) (FIG. 4). Moreover, median overall survival (40 days; 95% CI, 31, NA; P<0.001; n=11) was significantly longer for WP1193+IFN-α combinatorial therapy than for IFN-α alone (P<0.02). For the mice treated with the combinatorial therapy of WP1193 and IFN-α, 27% survived long term (>84 days) (P<0.001 compared with the control group), and there was at least a 135% increase in median survival time when the experiment was terminated to perform the tumor rechallenge experiments. To determine whether mice with intracerebral tumors treated with both WP1193 and IFN-α were able to generate long-lasting protective immune memory, mice that survived for 84 days after the initial tumor cell implantation were re-inoculated with B16 cells in the contralateral hemisphere. Upon rechallenge, in the animal group that had received the WP1193 and IFN-α, the median survival time was 16 days, which did not differ significantly from the median survival time (17 days) of naïve, control mice. There were no long-term survivors in the rechallenged group (data not shown), indicating that long-lasting immune memory was not induced by the combination therapy.

Therefore, the combination a STAT3 inhibitor such as WP1193 together with IFN-α enhances both NK and CD8+ cytotoxicity by enhancing pro-inflammatory cytokines and can be a treatment modality for melanoma patients with CNS disease who currently have very few therapeutic options available and who are typically excluded from clinical trials.

### Example 65

### IFN-α Inhibits In Vivo Death Secondary to LMD

Because IFN-α has previously been demonstrated to inhibit invasion and melanoma LMD in clinical trials, we assessed at the time of treatment failure the etiology of the animal's death whose bodies could be recovered for autopsy. Both the control group (n=8) and in those treated with a sub-therapeutic dose of WP1193 (n=9) had macroscopic evidence of LMD, 75% and 67%, respectively, (Data not shown). In contrast, in those mice treated with IFN-α (n=6) or the combination of IFN-α and WP1193 (n=4), only 17% in the former and none of the animals treated with the combination died of LMD (FIG. 5). As would be expected, in a sub-analysis within each treatment group, mice that developed LMD died sooner than did those that died of tumor. Specifically, in the control group, median survival was 16.7±0.7 days in those mice that developed LMD and 18.5±1.5 days in those that died of tumor. Furthermore, in the sub-therapeutic WP1193 group, median survival was 17.7±0.3 days in mice that developed LMD and 20±2.1 days in those that died of tumor. In contrast, within the IFN-α treatment group, the median survival of mice with progressive tumor was 25.8±2.5 days, which was further increased to 29.7±5.5 days in the combination treatment group. Within individual treatment groups there was insufficient power to draw statistically meaningful conclusions; however when we assessed all mice who died of LMD, the median survival was 16.9±0.4 days compared with 24.7±2 days for mice dying of tumor progression (P=0.0006), indicating this model system conforms to the negative prognostic influence of LMD observed in human patients.

### Example 66

### In Vivo Effects of IFN-α and WP1193, and in Combination on Tregs

In vivo treatment of mice with IFN-α and WP1193 inhibits bone marrow-derived Tregs but combinational therapy is not synergistic. To ascertain the in vivo effects of IFN-α and WP1193, and in combination on Tregs, non-tumor bearing mice were treated for 16 days. Both WP1193 and IFN-α significantly inhibited the number of Tregs (CD4+Foxp3+) in the bone marrow by 31% and 78%, respectively, compared with the control (P<0.05 and P<0.01; Table E1).

TABLE E1

| Sample | % of CD4+ Foxp3+ Cells |
|---|---|
| Control | 24.5 |
| WP1193 | 16.8 |
| IFN alpha | 5.2 |
| IFN alpha + WP1193 | 7.0 |

WP1193 and IFN-α also significantly inhibited the number of Tregs (CD4+Foxp3+) in the peripheral blood by 20% and 46%, respectively, compared with the control (P<0.05; data not shown). However, the combination of IFN-α and WP1193 was not additive or synergistic for inhibiting the number of Tregs in either the bone marrow or the blood. Furthermore, WP1193 or IFN-α alone or in combination did not inhibit the number of Foxp3+ Tregs in the thymus, lymph nodes, or spleen (data not shown). This suggested to us that an additive inhibition of Tregs was not the underlying mechanism observed for the in vivo activity of the combination of WP1193 and IFN-α.

In additional experiments, CD4⁺CD25⁻CD62L^{hi} naïve T cells from C57BL/6J mice were stimulated by plate-bound anti-CD3 (2 μg/ml) and soluble anti-CD28 (2 μg/ml) in the presence of TGF-β1 (1 ng/ml) and hIL-2 (200 U/ml), with 0, 0.1, and 1.0 μM WP1066 for inducible Tregs (iTreg) differentiation. CD4⁺CD25⁺ T cells (natural Tregs, nTreg) were stimulated by plate-bound anti-CD3 (2 μg/ml) and soluble anti-CD28 (2 μg/ml) in the presence of hIL-2 (200 U/ml), with 0, 0.1, and 1.0 μM WP1066. Ninety-six hours after stimulation, the cells were analyzed for intracellular FoxP3 expression by flow cytometry. The percentage of FoxP3 expressing T cells is shown in FIG. 2.

### Example 67

### In Vivo Treatment of Mice with IFN-α or WP1193 Enhances Melanoma Immune-Mediated Cytotoxicity

To ascertain the underlying in vivo immunological mechanism that is responsible for the therapeutic efficacy, we examined splenocytes cytotoxic responses directed against B16 melanoma cells. Splenocytes from control mice and mice treated with WP1193, IFN-α, or IFN-α+WP1193 were isolated and co-cultured with CFSE-labeled B16 target cells for 48 hours to assess splenocyte cytotoxicity against B16 cells. In both ratios of splenocyte effector cells to B16 target cells (30:1 and 100:1), the splenocytes from the WP or IFN-α-treated mice had significantly increased cytotoxic clearance of the B16 target cells compared with control mice (P<0.05; Table E2). Furthermore, in mice treated with both IFN-α and WP1193, there was additive enhanced cytotoxic clearance of the B16 target cells compared with mice that were treated with either WP1193 or IFN-α alone (P<0.01;).

TABLE E2

| | |
|---|---|
| Sample | % of B16 Killimg |

30:1 ratio of splenocytes to B16 Tumor Cells

| | |
|---|---|
| Control | 25.2 |
| WP1193 | 29.8 |
| IFN alpha | 40.1 |
| IFN alpha + WP1193 | 59.7 |

100:1 ratio of splenocytes to B16 Tumor Cells

| | |
|---|---|
| Control | 38.1 |
| WP1193 | 42.0 |
| IFN alpha | 51.6 |
| IFN alpha + WP1193 | 80.1 |

To further ascertain the underlying immunological mechanism that is responsible for the therapeutic efficacy, we performed cytotoxic responses directed against B16 melanoma cells by NK cells and CD8+ cells. The NK1.1+CD3− (NK) cells and CD3+CD8+ T cells from spleens of 4- to 6-week-old mice were isolated, co-cultured with CFSE-labeled B16 target cells and treated with RPMI 1640 medium (control), WP1193 (2 μM), IFN-α (20 ng/ml), or IFN-α (20 ng/ml)+WP1193 (2 μM) for 48 h to assess NK or T cell cytotoxicity against B16 cells. Both the NK and CD8+ T cell populations from the WP1193 (P=0.01 and P=0.02, respectively) or IFN-α-treated group (P=0.0002 and P=0.0006, respectively) increased cytotoxic clearance of the B16 target cells compared with the control group. Furthermore, there was enhanced cytotoxic clearance of the B16 target cells in the NK cells or CD8+ T cells treated with both IFN-α and WP1193 compared with the NK cells or CD8+ T cells treated with either WP1193 or IFN-α alone (P<0.01; Table E3).

TABLE E3

| | |
|---|---|
| Sample | % of B16 Killimg |

NK Cells

| | |
|---|---|
| Control | 8.8 |
| WP1193 | 10.9 |
| IFN alpha | 12.5 |
| IFN alpha + WP1193 | 17.1 |

CD8+ T cells

| | |
|---|---|
| Control | 10.8 |
| WP1193 | 11.2 |
| IFN alpha | 14.3 |
| IFN alpha + WP1193 | 23.4 |

This experiment was reproduced twice in its entirety with identical findings.

### Example 68

### MHC I and NK Activating Ligands are Expressed on Melanoma Cells but are not Further Enhanced by Combination Therapy

Because we observed NK and CD8+ T cell-mediated anti-tumor cytotoxicity were enhanced with combinational therapy, to ascertain if either IFN-α, WP1193 or both were augmenting the expression of MHC or NK activating receptors or their ligands, splenocytes and B16 cells were treated with RPMI 1640 medium (control), WP1193 (2 μM), IFN-α (20 ng/ml), or IFN-α (20 ng/ml)+WP1193 (2 μM) for 24 hours. The NK-activating ligands (Rae-1, H60 and CD155) and MHC (I and II) on B16 cells and the NK-activating receptors (NKG2D, KLRD1, NKp46 and DNAM-1) on NK1.1+ NK cells were analyzed by flow cytometric analysis.

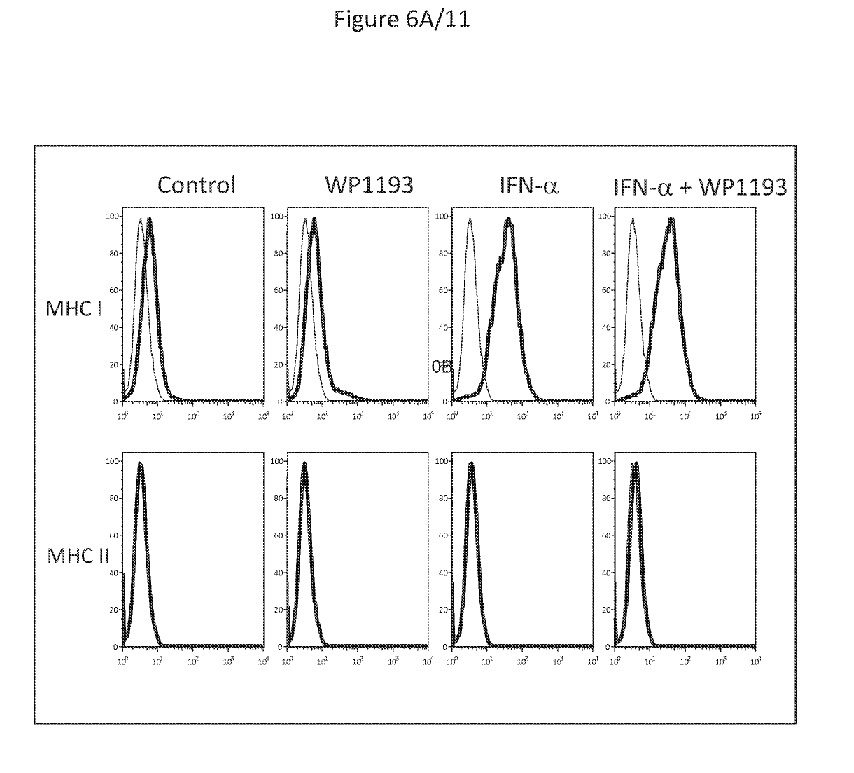

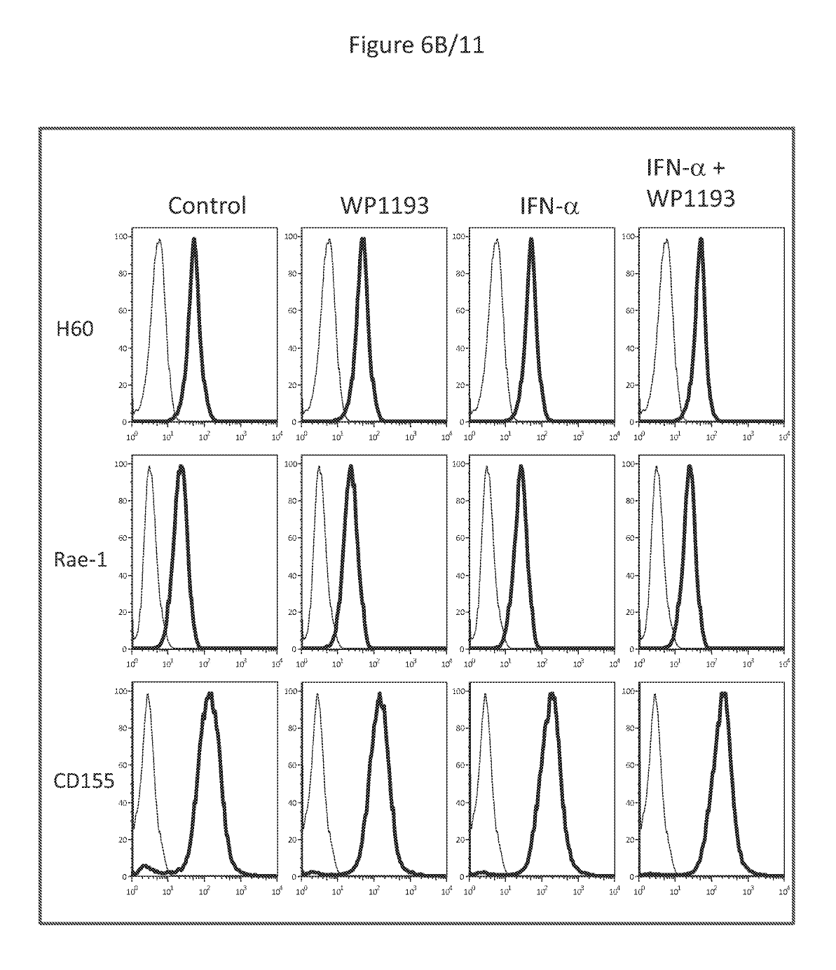

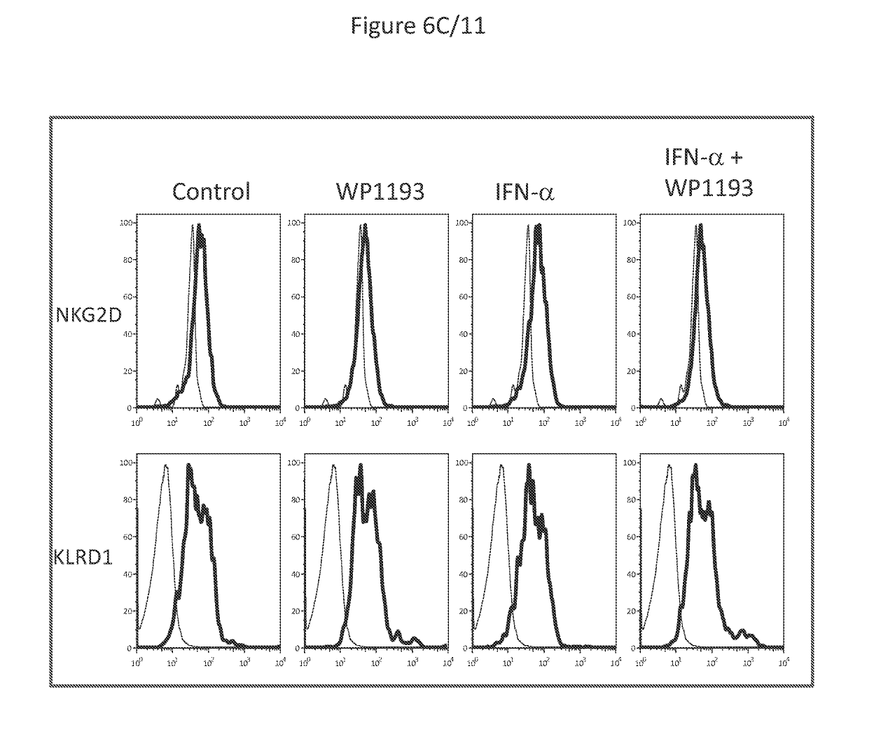

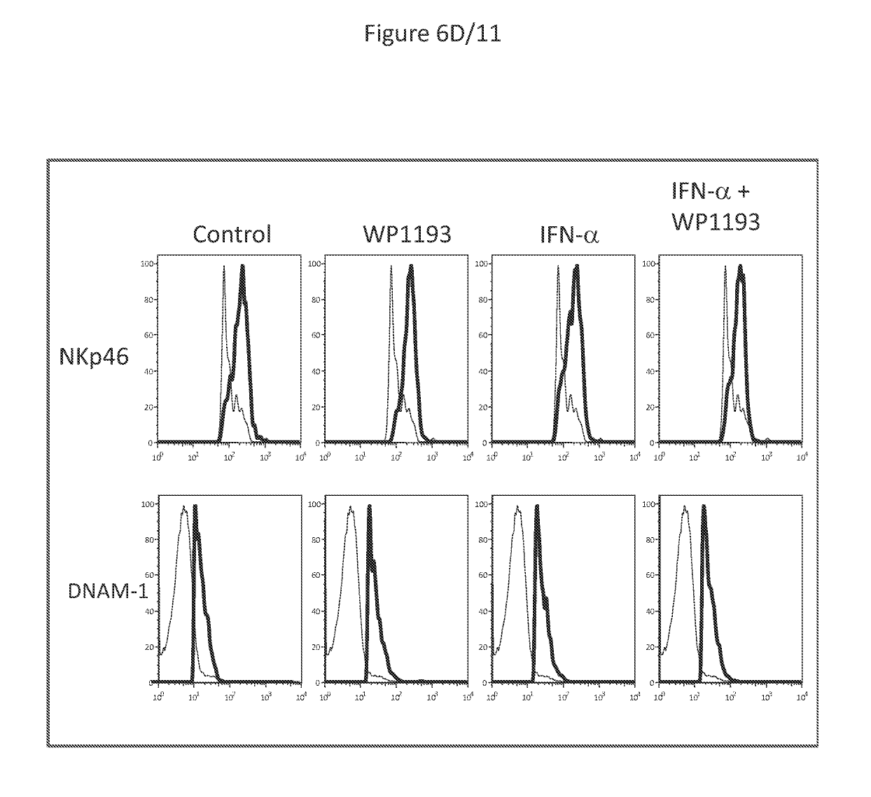

MHC I but not MHC II was expressed on B16. IFN-α enhanced MHC I expression but this was not further enhanced with WP1193 (FIG. 6A). Additionally, B16 expressed H60, Rae-1 and CD155; however, neither IFN-α nor the WP1193 treatment altered the mean fluorescent intensity on the surface indicating that these treatments do not alter the receptor density of the NK ligands (FIG. 6B). Furthermore, NKG2D, KLRD1, NKp46 and DNAM-1 were expressed on the NK cells but also did not appear to be up-regulated by either WP1193 or IFN-α indicating that these treatments do not alter the receptor density of the NK receptors (FIGS. 6C and 6D).

### Example 69

### In Vivo Treatment of Mice with IFN-α or WP1193 Alone or in the Combination Enhances Cytokine Production

Because B16 cells express NK-activating ligands and the combinational approach enhanced NK-mediated anti-tumor cytotoxicity, we next examined whether WP1193 or IFN-α individually or in the combination affected key cytokines, and specifically IFN-γ, which enhances NK cytotoxicity by increasing the levels of expression of TRAIL and Fas ligand.

C57BL/6J mice were treated as described in the Tables and serum was collected for the measurement of IL-1β, IL-2, IL-4, IL-10, IL-12, TNF-α and IFN-α concentrations using the Bio-Plex Promouse Cytokine 8-plex assay. Serum concentrations of IL-2 and IL-4 from mice in all treatment groups were below the detection level (sensitivity) of the assay. Combination therapy of IFN-α and WP1193 markedly enhanced the production of IL-1β and IFN-γ (Table E4) compared with untreated controls, indicating that these cytokines may be contributing to therapeutic efficacy. IFN-α modestly enhanced serum concentrations of TNF-α (Table E4). Serum levels of the other cytokines were not significantly enhanced by the combination of IFN-α and WP1193 (data not shown). This experiment was reproduced twice in its entirety with identical findings.

TABLE E4

| Sample | IL-1B Concentration (pg/ml) |
|---|---|
| Control | 40.1 |
| WP1193 | 20.0 |
| IFN alpha | 41.3 |
| IFN alpha + WP1193 | 101.2 |

IFN-gamma Concentration (pg/ml)

| | |
|---|---|
| Control | 1.5 |
| WP1193 | Not detected |
| IFN alpha | 20.2 |
| IFN alpha + WP1193 | 51.1 |

TNF alpha Concentration (pg/ml)

| | |
|---|---|
| Control | 60.5 |
| WP1193 | 60.0 |
| IFN alpha | 98.7 |
| IFN alpha + WP1193 | 87.4 |

IFN-γ has been shown to markedly promote both NK and cytotoxic T cell activity, to increase the expression of MHC, and enhance antigen expression. Palmer K J, Harries M, Gore M E, Collins M K, Interferon-Alpha (IFN-Alpha) Stimulates Anti-Melanoma Cytotoxic T Lymphocyte (CTL) Generation In Mixed Lymphocyte Tumour Cultures (MLTC), Clin Exp Immunol 2000; 119:412-8; Carballido J A, Molto L M, Manzano L, Olivier C, Salmeron O J, Alvarez de Mon M, Interferon-Alpha-2b Enhances The Natural Killer Activity Of Patients With Transitional Cell Carcinoma Of The Bladder, Cancer 1993; 72:1743-8; Kaser A, Enrich B, Ludwiczek O, Vogel W, Tilg H, Interferon-Alpha (IFN-α) Enhances Cytotoxicity In Healthy Volunteers And Chronic Hepatitis C Infection Mainly By The Perforin Pathway, Clin Exp Immunol 1999; 118:71-7; Keir M E, Stoddart C A, Linquist-Stepps V, Moreno M E, McCune J M, IFN-α Secretion By Type 2 Predendritic Cells Up-Regulates MHC Class 1 In The HIV-1-Infected Thymus, J Immunol 2002; 168:325-31; Greiner J W, Fisher P B, Pestka S, Schlom J, Differential Effects Of Recombinant Human Leukocyte Interferons On Cell Surface Antigen Expression, Cancer Res 1986; 46:4984-90.

The enhancement of some immune stimulatory cytokines such as TNF-α and IFN-γ can also exert direct cytotoxic tumor effects and both of these were induced with IFN-α and WP1193 in vivo; thus, it is possible that the TNF-α and IFN-γ also exerted direct effects on the intracerebral B16 and could be participating in the observed in vivo efficacy.

### Example 70

### Selective Activation of STAT3 by IFN Alpha and IFN Beta in Human Hepatocarcinoma Cells, (HH), HuT78 and MJ Cutaneous T Cell Lymphoma (CTCL) Cells

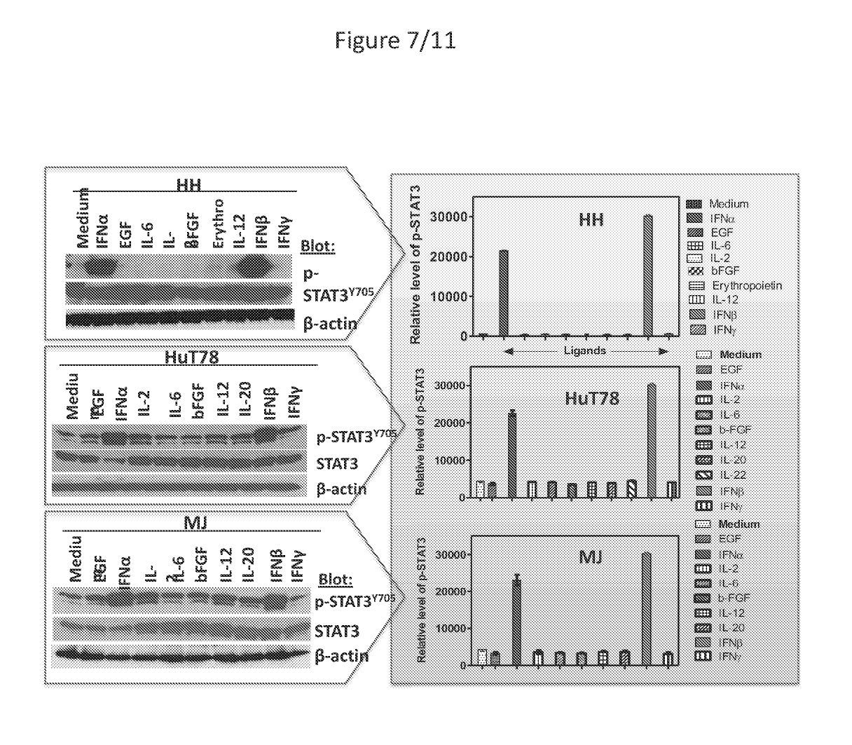

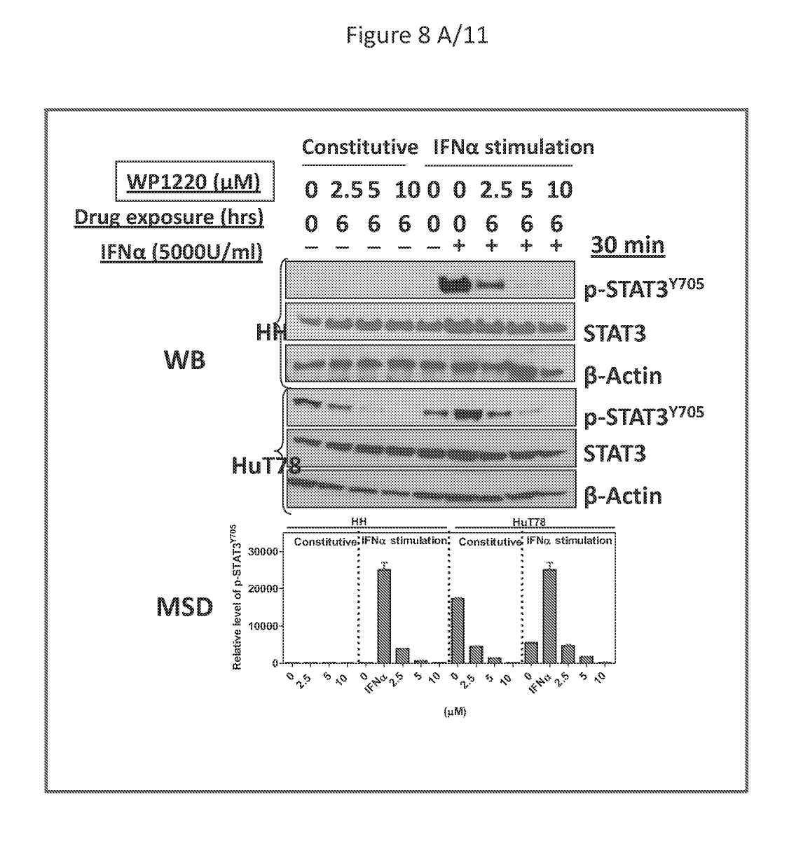

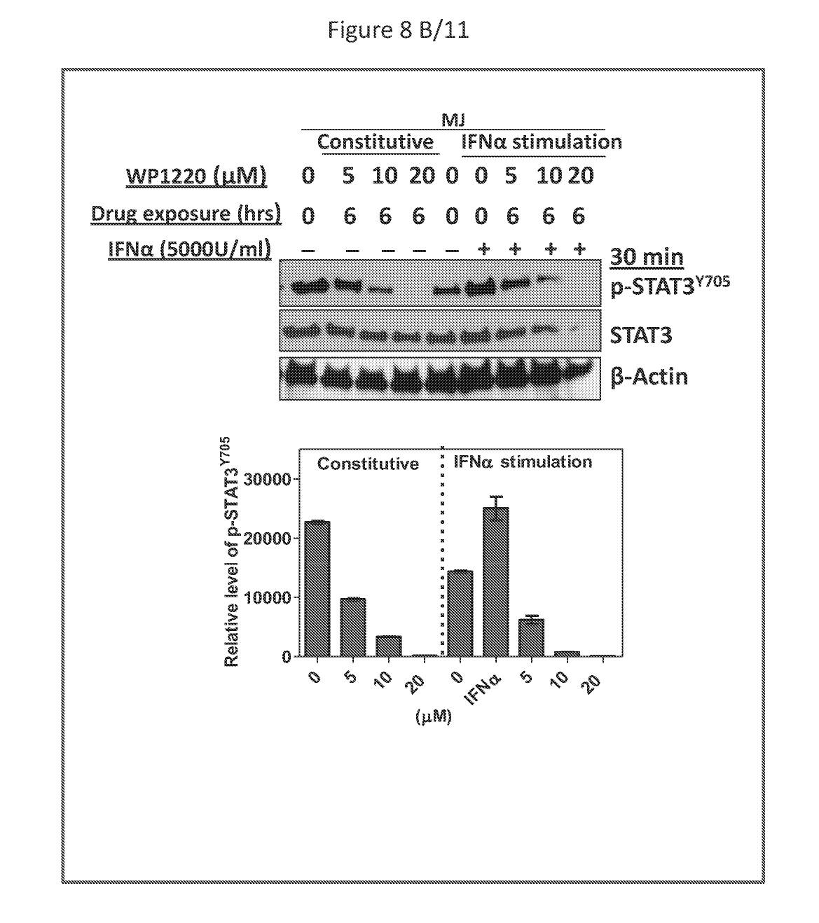

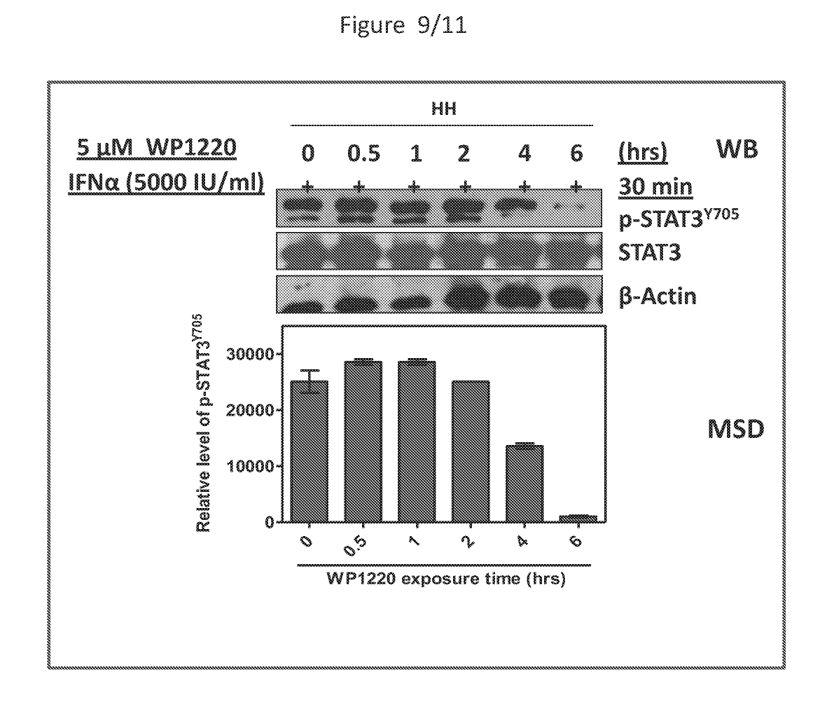

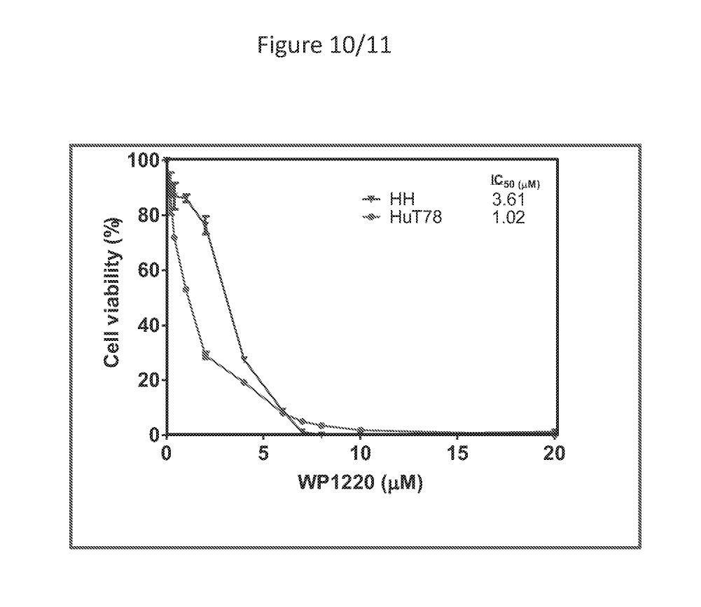

To determine whether IFN alpha and beta were able to selectively phosphorylate STAT3 in other cell types. Studies were conducted in a range of other biologically relevant cell types. In these experiments, cells were grown in media supplemented with 10% FBS, and then serum starved for 18 hours and exposed to various stimuli for 30 minutes. The results shown in FIG. 7, demonstrate that IFN-α and IFN-β selectively stimulates tyrosine phosphorylation of STAT3 in HH, HuT78 and MJ CTCL lines to specifically form pSTAT3 phosphorylated at tyrosine 705. By comparison the other agents showed significantly less, or no detectable levels of phosphorylation of pSTAT3 at tyrosine 705. Furthermore the phosphorylation of constitutive, and interferon stimulated, and constitutive phosphorylation of STAT3 was specifically blocked by WP1220 in these cells (FIG. 8A, and B)). Moreover, exposure of HH cells to WP1220 cells also resulted in a time dependent decrease in the relative level of activated pSTAT3 (FIG. 9). This compound was also capable of potently inhibiting the in vitro growth of HuT78 and HH CTCL cell lines. (FIG. 10), and induced caspase-3 and PARP cleavage in these cells (Table E5)

TABLE E5

| | |
|---|---|
| HH cells | HuT78 Cells |

| Cells | Cleaved | Cleaved | Cleaved | Cleaved |
|---|---|---|---|---|
| Conditions | Caspase | PARP | Caspase | PARP |
| DMSO (0.05%) | + | +/− | + | +/− |
| 5.0 uM WP1220 | ++++ | +++++ | ++++ | ++++ |
| 10.0 uM WP1220 | ++++ | +++ | +++ | +++ |

Treatment of the HH, HuT78 and MJ CTCL cell lines with WP1220 also resulted in the specific induction of caspase-3 activity in these cells (Table E6) Results represent means based on duplicate samples (for cleaved caspase, ++++=60,000-40,000; +=0-20,000; for Cleaved PARP, +++++=10,000-7,000; ++++=7,000-5,000; +++=5,000-2000; +/−=less than 1,000).

TABLE E6

| Cells | HH cells | HuT78 Cells | MJ cells |
|---|---|---|---|
| DMSO (0.05%) | +/− | +/− | +/− |
| 10.0 uM WP1220 | +++++ | ++++ | +++ |

HH, HuT78 and MJ cells were treated with vehicle alone or WP1220 for 24 hours. Total caspase-3 and cleaved caspase-3 levels were measured in 15 ug lysates by a meso-Scale 2400 imager. Results represent means fold stimulations compared to the controls based on duplicate samples (+++++=15-25 fold; ++++=10-15 fold; +++=5-10 fold; =/−=less than 2 fold.

In the SUP-T1 lymphoma cell line, an analysis of signalling effects of selected cytokines showed that IFN-α and IFN-β potently induce STAT3 but not Jak2 phosphorylation, indicating that activation of STAT3 by IFN-α and IFN-β is not mediated by Jak2 (data not shown).

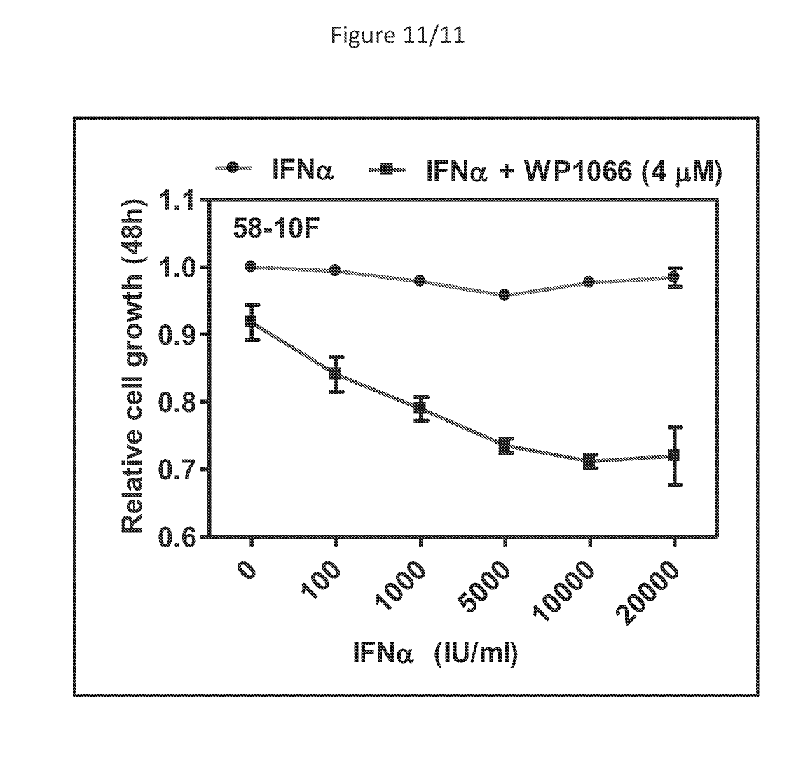

To determine whether the combination of IFN and a STAT3 inhibitor was also capable of inhibiting other tumor cells, studies were also performed with ependymoma 58-10F tumor cells. The results (FIG. 11) showed that the combination of the STAT3 inhibitor WP1066 and IFN a was able to more potentently inhibit cell growth than either mono therapy alone.

### Example 71

### Selective Activation of STAT3 by IFN Alpha and IFN Beta in Human HaCaT Keratinocytes

To determine whether IFN-α and IFN-β were capable of inducing STAT3 activation in keratinocytes, HaCaT cells were grown in DMEM supplemented with 10% FBS. Prior to experiments, the DMEM medium was changed to KGM-SFM containing rEGF and bovine pituitary extract for 12 hours, and the cells then exposed to various stimuli for 30 minutes. Cell lysates were extracted in IP lysis buffer were diluted in MSD extraction buffer and 25 uL of the cell lysate containing 10 ug protein was used in the analysis via chemiluminescent detection (MSD Technology). To test for inhibition by WP1220 (WP), cells were pre-incubated with WP1220 prior to the addition of IFN α. The results are shown in Table E7.

TABLE E7

| | Relative level |
|---|---|
| Conditions | of pSAT3 |
| Serum | + |
| SFM | + |
| IFN α | +++++ |
| EGF | + |
| IL-6 | +++++ |
| IL-2 | + |
| bFGF | + |
| Erythropoietin | + |

Results represent means fold stimulations compared to the controls based on duplicate samples (+++++=400-500 fold; +=less than 200 fold.

TABLE E8
Inhibition by WP1220

| | |
|---|---|
| SFM + IFN α | +++++ |
| IFN α + WP (5 uM) | +++ |
| IFN α + WP (10 uM) | ++ |
| IFN α + WP (20 uM) | + |

Results represent mean stimulations compared to the controls based on duplicate samples (+++++=10,000-6,000; +++=2,000-4,000; ++=2,000-1,500; +=less than 1,500.

The especially high selectivity of IFN-α and IFN-β toward STAT3 activation which was observed in CTCL tumors, where none of the other usually potent activators of STAT3, including IL-6, were able to induce STAT3 phosphorylation suggests that inhibiting IFN-induced STAT3 activation will potentiate the activity of IFNs. For example, we demonstrated that the combination of IFN-α and p-STAT3 blockade can exert efficacy against intracerebral established CNS melanoma. Patients with CNS melanoma, especially those with LMD, are typically refractory to currently available standard therapies and our preclinical data would suggest that this combination might have clinical utility.

This is notable considering that immunotherapeutic approaches for melanoma have been disappointing. Rosenberg S A, Yang J C, Restifo N P, Cancer Immunotherapy: Moving Beyond Current Vaccines, Nat Med 2004; 10:909-15. The cytokine IFN-α is currently FDA-approved for patients with stage III melanoma; however, only 15% of melanoma patients have an objective response. Agarwala S S, Kirkwood J M, Interferons in Melanoma, Curr Opin Oncol 1996; 8:167-74.

It is concluded, based on these studies that the use of IFN-α as an anticancer drug will lead to activation of STAT3 and in part might induce proliferation and survival of tumor cell as well as induce angiogenesis and metastasis thus the combination of IFN-α with STAT3 inhibitors should increase the effectiveness of IFN-α therapy.

Secondly, the use of IFN-α as a drug for non-cancer indications (including, for example, antiviral therapy) will lead to activation of STAT3 and consequently to outburst of psoriasis. This can be prevented by cotreatment with p-STAT3 inhibitors, as well as Jak2 inhibitors.

What is claimed is:

1. A method of treating a proliferative disease comprising administering to a patient a therapeutically effective amount of Type 1 interferon in combination with a STAT3 pathway inhibitor.

2. The method of claim 1, wherein the STAT3 pathway inhibitor has structural Formula I: 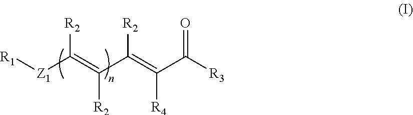
or a pharmaceutically acceptable salt thereof, wherein:
n is 0 or 1;
m is an integer selected from 1, 2, 3, or 4;
R₁ is selected from the group consisting of: 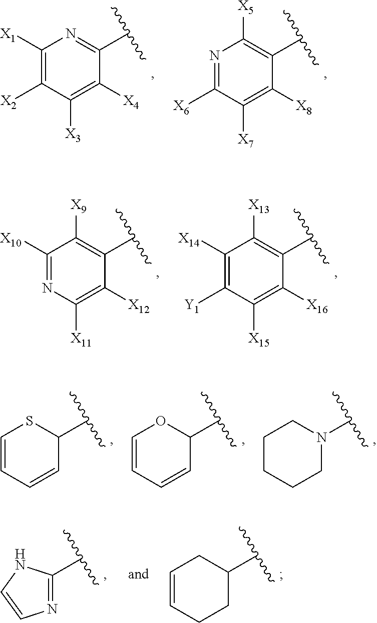
each instance of R₂ is independently selected from the group consisting of alkyl, alkenyl, alkoxy, arylalkyl, halogen, hydrogen, hydroxyl, nitro, thiol, mercaptan, amino, and alkylamino;
R₃ is selected from the group consisting of: 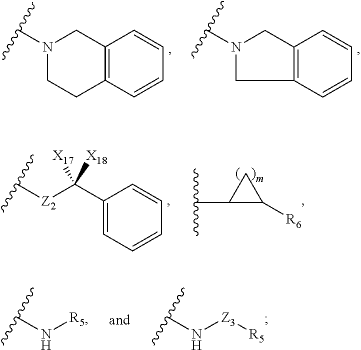
R₄ is selected from the group consisting of cyano, alkylamine, CH₂S-alkyl, alkyl, and CH₂N₃;
R₅ and R₆ are each independently selected from the group consisting of: 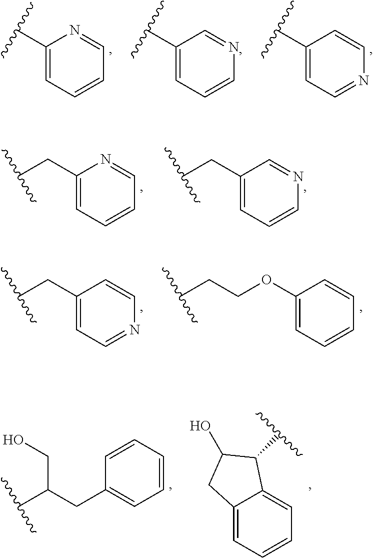
monosaccharide, polysaccharide, monosaccharide derivative, optionally substituted aryl, and optionally substituted arylalkyl;
X₁, X₂, X₃, X₄, X₅, X₆, X₇, X₈, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;
X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;
Y₁ is selected from the group consisting of hydroxyl, halogen, and nitro;
Z₁ is selected from the group consisting of alkyl and a bond;
Z₂ is selected from the group consisting of N H, S, and O; and
Z₃ is alkyl.

3. The method of claim 2, wherein:
R₁ is selected from the group consisting of: 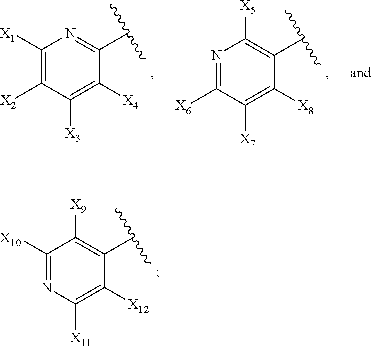
each instance of R₂ is hydrogen;
R₃ is 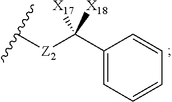
and
Z₂ is NH.

4. The method of claim 3, wherein:
X₁, X₂, X₃, X₄, X₅, X₆, X₇, X₈, X₉, X₁₀, X₁₁, and X₁₂ are each independently selected from the group consisting of hydrogen and halogen; and
X₁₇ and X₁₈ are each independently selected from the group consisting of hydrogen, alkyl, and cycloalkyl.

5. The method of claim 4, wherein:
R₁ is 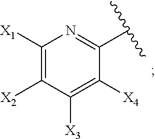
X₁ is halogen; and
X₂, X₃, and X₄ are hydrogen.

6. The method of claim 5, wherein:
one of X₁₇ and X₁₈ is hydrogen;
the other of one of X₁₇ and X₁₈ is selected from the group consisting of hydrogen, methyl, ethyl, and cyclopropyl.

7. The method of claim 6, wherein n is 0.

8. The method of claim 6, wherein n is 1.

9. The method of claim 1, wherein the STAT3 pathway inhibitor is selected from the group consisting of examples 1-63.

10. The method of claim 1, wherein the proliferative disease is selected from the group consisting of psoriasis, skin cancer, CNS cancer including brain cancer and cancer metastatic to CNS, ovarian cancer, head cancer and neck cancer, prostate cancer, hematological malignancies including leukemia, lymphoma and myeloma, and breast cancer.

11. The method of claim 1, wherein the combination of the STAT3 inhibitor and the Type 1 interferon is characterized by a synergistic response compared to either agent alone.

12. The method of claim 10, wherein the proliferative disease is skin cancer.

13. The method of claim 11, wherein the skin cancer is selected from the group consisting of squamous cell carcinomas, basal cell cancers, cutaneous T-cell lymphomas, primary cutaneous B cell lymphomas, Dermatofibrosarcoma protuberans, Merkel cell carcinoma, Kaposi's sarcoma, keratoacanthoma, and melanoma.

14. The method of claim 10, wherein the proliferative disease is melanoma.

15. The method of claim 14, wherein the melanoma is CNS melanoma.

16. The method of claim 14, wherein the patient has Leptomeningeal disease (LMD).

17. The method of claim 14, wherein the patient has stage III melanoma.

18. The method of claim 14, wherein the patient has stage IV melanoma.

19-26. (canceled)

27. The method of claim 9, wherein the STAT3 pathway inhibitor has a structural formula selected from the group consisting of:

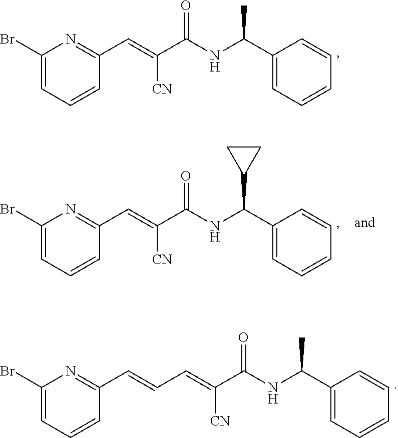 [SMILES: C[C@H](NC(=O)/C(C#N)=C/c1cccc(Br)n1)c1ccccc1 ; N#C/C(=C\c1cccc(Br)n1)C(=O)N[C@H](c1ccccc1)C1CC1 ; C[C@H](NC(=O)/C(C#N)=C/C=C/c1cccc(Br)n1)c1ccccc1]

28. A method of potentiating the activity of Type 1 interferon for treatment of a proliferative disease comprising administering to a patient a therapeutically effective amount of Type 1 interferon in combination with a STAT3 pathway inhibitor.

29. The method of claim 28, wherein said STAT3 pathway inhibitor has structural Formula I: 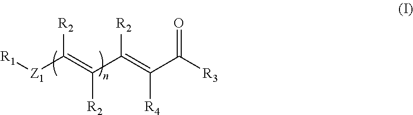
or a pharmaceutically acceptable salt thereof, wherein:
n is 0 or 1;
m is an integer selected from 1, 2, 3, or 4;
R₁ is selected from the group consisting of: 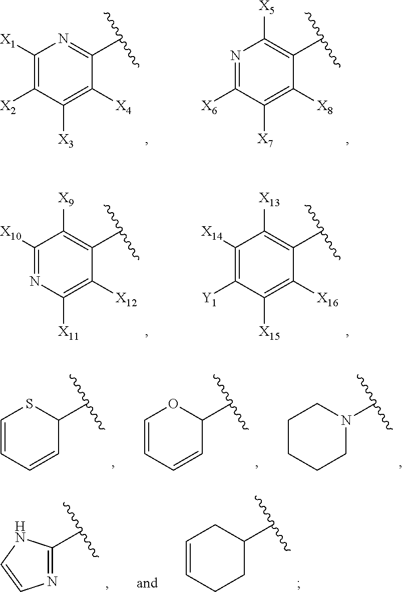
each instance of R₂ is independently selected from the group consisting of alkyl, alkenyl, alkoxy, arylalkyl, halogen, hydrogen, hydroxyl, nitro, thiol, mercaptan, amino, and alkylamino;
R₃ is selected from the group consisting of: 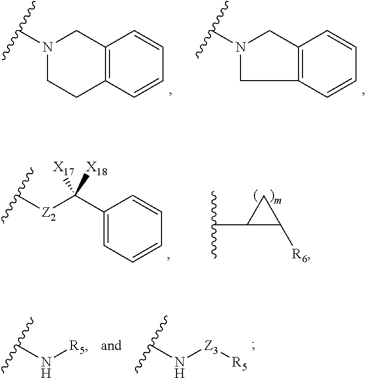
R₄ is selected from the group consisting of cyano, alkylamine, CH₂S-alkyl, alkyl, and CH₂N₃;
R₅ and R₆ are each independently selected from the group consisting of: 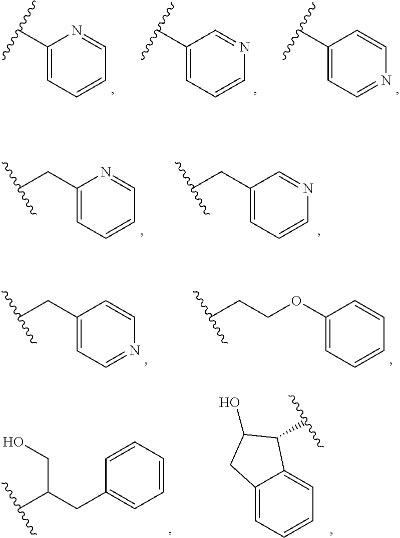
monosaccharide, polysaccharide, monosaccharide derivative, optionally substituted aryl, and optionally substituted arylalkyl;
X₁, X₂, X₃, X₄, X₅, X₆, X₇, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;
X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃);
Y₁ is selected from the group consisting of hydroxyl, halogen, and nitro;
Z₁ is selected from the group consisting of alkyl and a bond;
Z₂ is selected from the group consisting of N H, S, and O; and
Z₃ is alkyl.

30. The method of claim 28, wherein the proliferative disease is selected from the group consisting of psoriasis, skin cancer, CNS cancer including brain cancer and cancer metastatic to CNS, ovarian cancer, head cancer and neck cancer, prostate cancer, hematological malignancies including leukemia, lymphoma and myeloma, and breast cancer.

31-48. (canceled)

49. The method of claim 29, wherein the STAT3 pathway inhibitor has a structural formula selected from the group consisting of:

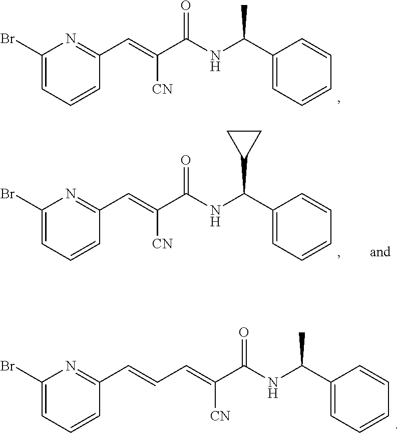

50. A method of modulating IFN-induced STAT3 activation during anti-viral therapy with a type 1 interferon, comprising administering to a patient a therapeutically effective amount of Type 1 interferon in combination with a STAT3 pathway inhibitor, wherein the STAT3 pathway inhibitor reduces the severity of at least one side effect of the Type 1 interferon.

51. The method of claim 50, wherein the STAT3 pathway inhibitor as structural Formula I: 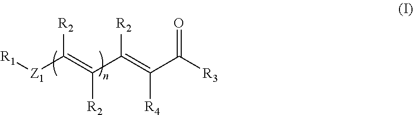
or a pharmaceutically acceptable salt thereof, wherein:
n is 0 or 1;
m is an integer selected from 1, 2, 3, or 4;
R₁ is selected from the group consisting of: 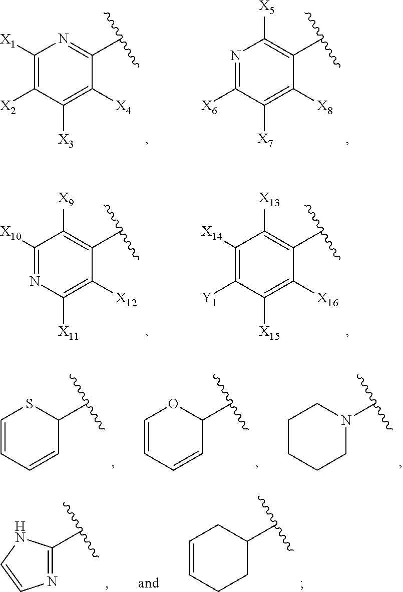
each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;
R₃ is selected from the group consisting of: 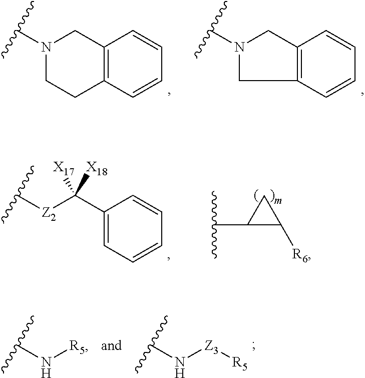
R₄ is selected from the group consisting of cyano, alkylamine, CH₂S-alkyl, alkyl, and CH₂N₃;
R₅ and R₆ are each independently selected from the group consisting of: 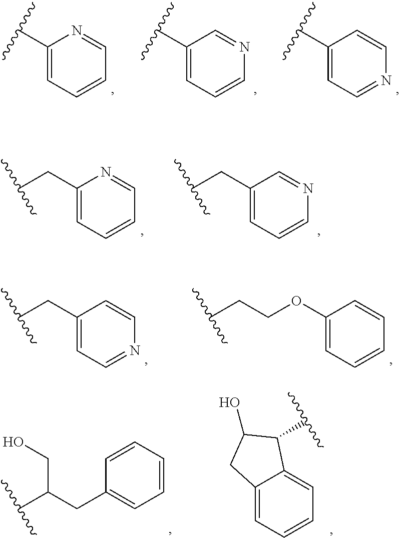
monosaccharide, polysaccharide, monosaccharide derivative, optionally substituted aryl, and optionally substituted arylalkyl;
X₁, X₂, X₃, X₄, X₅, X₆, X₇, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;
X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;
Y₁ is selected from the group consisting of hydroxyl, halogen, and nitro;
Z₁ is selected from the group consisting of alkyl and a bond;
Z₂ is selected from the group consisting of N H, S, and O; and
Z₃ is alkyl.

52-59. (canceled)

60. The method of claim 51, wherein the STAT3 pathway inhibitor has a structural formula selected from the group consisting of:

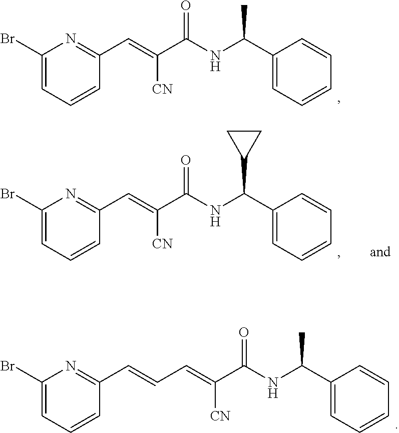

61. A method of modulating IFN-induced STAT3 activation during treatment for viral hepatitis comprising administering to a patient a therapeutically effective amount of Type 1 interferon in combination with a STAT3 pathway inhibitor.

62. The method of claim 61, wherein the STAT3 pathway inhibitor has structural Formula I: 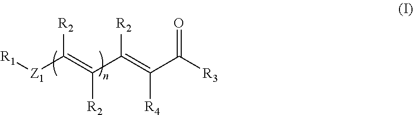
or a pharmaceutically acceptable salt thereof, wherein:
n is 0 or 1;
m is an integer selected from 1, 2, 3, or 4;
R₁ is selected from the group consisting of: 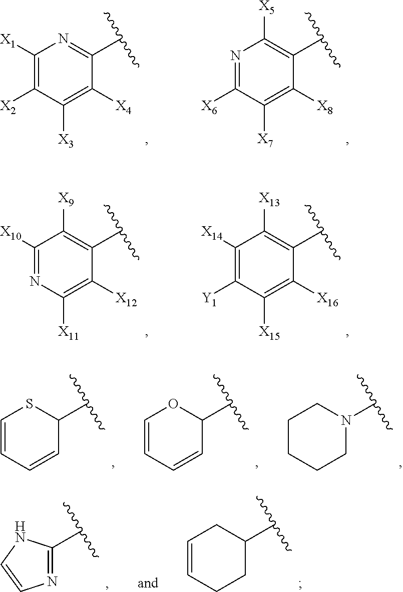
each instance of R₂ is independently selected from the group consisting of alkyl, alkenyl, alkynyl, alkoxy, arylalkyl, halogen, hydrogen, hydroxyl, nitro, thiol, mercaptan, amino, and alkylamino;
R₃ is selected from the group consisting of: 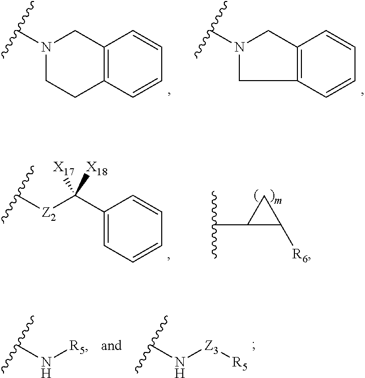
R₄ is selected from the group consisting of cyano, alkylamine, CH₂S-alkyl, alkyl, and CH₂N₃;
R₅ and R₆ are each independently selected from the group consisting of: 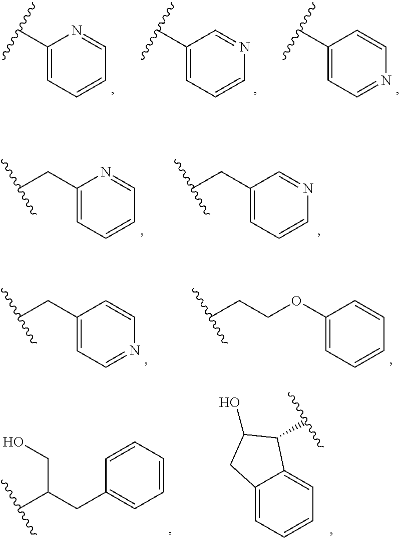
monosaccharide, polysaccharide, monosaccharide derivative, optionally substituted aryl, and optionally substituted arylalkyl;
X₁, X₂, X₃, X₄, X₅, X₆, X₇, X₉, X₁₀, X₁₁, X₁₂, X₁₃, X₁₄, X₁₅, and X₁₆ are each independently selected from the group consisting of hydrogen, halogen, alkyl, alkoxy, hydroxy, trihalomethyl, and nitro;
X₁₇ and X₁₈ are each independently selected from the group consisting of hydrogen, alkyl, aryl, alkoxy, aryloxy, cycloalkyl, aryl, arylalkyl, alkoxycarbonyl, alkoxycarbonylalkyl, acyl, hydroxyl, hydroxyalkyl, —CH₂OC(O)H₃, and —CH₂OC(O)C(CH₃)₃;
Y₁ is selected from the group consisting of hydroxyl, halogen, and nitro;
Z₁ is selected from the group consisting of alkyl and a bond;
Z₂ is selected from the group consisting of N H, S, and O; and
Z₃ is alkyl.

63. The method of claim 61, wherein the side effect of the Type 1 interferon is selected from the group consisting of atypical dermatitis, psoriasis, Crohn's disease, thyroiditis, autoimmune hepatitis, inflammatory bowel disease, and pulmonary fibrosis.

64-69. (canceled)

70. The method of claim 62, wherein the STAT3 pathway inhibitor has a structural formula selected from the group consisting of:

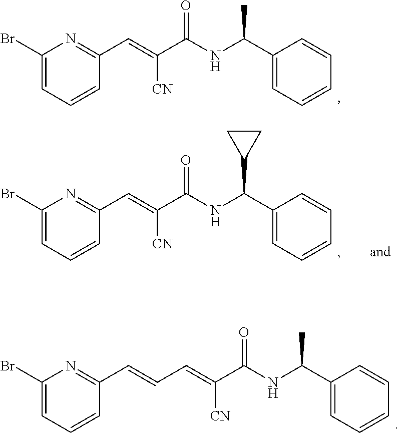

71. A method of modulating anti-viral therapy with a type I interferon, comprising the step of administering to a patient a therapeutically effective amount of Type 1 interferon in combination with a Jak2 inhibitor, wherein the Jak2 pathway inhibitor reduces the severity of at least one side effect of the Type 1 interferon.

72. The method of claim 71, wherein the side effect of the Type 1 interferon is selected from the group consisting of atypical dermatitis, psoriasis, Crohn's disease, thyroiditis, autoimmune hepatitis, inflammatory bowel disease, and pulmonary fibrosis.

73. The method of claim 72, wherein the Jak2 inhibitor is selected from the group consisting of INCB0118424, TG1O1348, CEP-701 (lestaurtinib), AZD1480, XLO19, CYT-387, SGI-1252, SB1518, tasocitinib (CP-690550), LY3009104 (INCB28050), AG490, Tkip, Z3, C7, and TGI01209.

74-102. (canceled)
